(19) United States
(12) Patent Application Publication
(10) Pub. No.: US 2011/0104103 A1
(43) Pub. Date: May 5, 2011

(54) Method of conjugating therapeutic compounds to cell targeting devices via metal complexes

(75) Inventors: Robert Jochem Heetebrij, Leiden (NL); Robbert Jan Kok, Nieuwegein (NL); Eduard Gerhard Talman, Diemen (NL); Klaas Poelstra, Buitenpost (NL); Grietje Molema, Groningen (NL)

(73) Assignee: KREATECH BIOTECHNOLOGY B.V., Amsterdam (NL)

(21) Appl. No.: 11/995,960

(22) PCT Filed: Jul. 20, 2006
(86) PCT No.: PCT/NL2006/000383

§ 371 Date: Jan. 20, 2011

(30) Foreign Application Priority Data
Jul. 20, 2005 (EP) 05076682.3

(51) Int. Cl. A61K 31/555; C07K 16/00; C07K 14/765; C07K 14/77; C07K 14/00; C07K 14/475; C07K 14/54; C12N 9/36; C12N 9/16; C12N 9/02; C07K 14/52; C07K 14/525; C07F 15/00; A61K 39/395; A61K 38/47; A61K 38/46; A61K 38/44; A61K 38/19; A61K 38/20; A61K 38/18; A61K 38/02; A61P 29/00; A61P 9/12

(52) U.S. Cl. 424/85.2; 530/387.1; 530/363; 530/367; 530/387.3; 530/400; 530/399; 530/351; 435/206; 435/196; 435/189; 544/225; 548/402; 546/2; 424/181.1; 424/133.1; 424/94.61; 424/94.6; 424/94.4; 424/85.1; 514/188; 514/186; 514/184; 514/7.6; 514/1.1

(57) ABSTRACT

The present invention relates to a cell-targeting complex comprising a targeting moiety and a deliverable compound, wherein said targeting moiety and said deliverable compound are joined by means of a (transition) metal ion complex having at least a first reactive moiety for forming a coordination bond with a reactive site of said targeting moiety and having at least a second reactive moiety for forming a coordination bond with a reactive site of said deliverable compound, and wherein said deliverable compound is a therapeutic compound.

### FIELD OF THE INVENTION

The present invention is in the field of drug targeting, and relates in particular to the targeting of drugs to selected cell populations and to pharmaceutical compositions comprising cell-targeting moieties.

### BACKGROUND OF THE INVENTION

Site-specific or targeted delivery of drugs is considered a valuable tool to improve the therapeutic efficacy and to reduce the toxicity of drugs. Whereas non-targeted drug compounds typically reach their intended target cells via whole-body diffusion and passive diffusion or receptor mediated uptake over the cell membrane, targeted drugs home-in and concentrate mainly at the targeted tissues. Consequently, targeted drugs require smaller dosages while still allowing the drug to reach therapeutically effective levels inside the target cells. Also, the preferred lipophilic character of non-targeted drugs, which facilitates their easy passage over the cell membrane and which feature is not always in agreement with other requirements of the drug, is less relevant to targeted drugs. The targeting of drugs to specific cells is therefore a conceptually attractive method to enhance specificity, to decrease systemic toxicity and to allow for the therapeutic use of compounds that are in principle less suitable or unsuitable as systemic drugs.

In general, drug delivery technologies are aimed at altering the interaction of the drug with the in vivo environment and achieve that objective by conjugation of the drug with other molecules, entrapment of the drug within matrices or particles or simply by co-administration with other agents. The net result is either drug targeting or enhanced drug transport across biological barriers such that its bioavailability is improved with a reduction of the incidence of clinical side effects in subjects. Drug targeting is achieved when an alteration in the drug's bio-distribution favours drug accumulation at the desired site, which site is usually remote from the administration site.

Cell-selective delivery of drugs can, in principle, be obtained by coupling drug molecules to targeting moieties (macromolecular carriers that contain a chemical moiety that is specifically recognised by target cells in the diseased tissue). However, at present such cell-specific drug targeting preparations are only scarcely available due to some major technological hurdles. Apart from the availability of suitable drugs and targeting molecules, the linkage between the therapeutic agent and the targeting device often poses significant problems. For instance, chemically reactive groups for conventional conjugation chemistry may be absent, or chemically reactive groups may be (abundantly) present, but covalent linkage may (irreversibly) inhibit the bioactivity of the coupled therapeutic agent.

### SUMMARY OF THE INVENTION

The present invention provides for an improved coupling of therapeutic compounds to targeting groups.

In a first aspect, the present invention provides a cell-targeting complex comprising a targeting moiety and a deliverable compound, wherein said targeting moiety and said deliverable compound are joined by means of a (transition) metal ion complex having at least a (first) reactive moiety for forming a coordination bond with a reactive site of said targeting moiety and having at least a (second) reactive moiety for forming a coordination bond with a reactive site of said deliverable compound, and wherein said deliverable compound is a therapeutic compound. In a preferred embodiment, the metal ion complex is a platinum complex. The platinum complex may be a trans-platinum complex or it may be a cis-platinum complex. The cis-platinum complex furthermore preferably comprises an inert bidentate moiety as a stabilising bridge. In yet another preferred embodiment, the (platinum) metal ion complex comprises a tridentate moiety as stabilising bridge. In another embodiment of said metal ion complexes, said complex comprises at least two (transition) metal ions, whether or not the same. The reactive sites on the targeting moiety and deliverable compound are capable of forming (coordination) bonds with the (transition) metal ion complex. In preferred embodiments the targeting moiety and/or the deliverable compound therefore comprise one or more sulphur-containing reactive sites and/or one or more nitrogen-containing reactive sites.

The targeting moiety preferably comprises a member of a binding pair. In preferred embodiments, the targeting moiety comprises an antibody or parts thereof (e.g. single chain, Fab-fragments), a receptor ligand. The binding pairs may be bonded directly to the metal ion complex, or via a spacer or linking molecule. A very suitable linking molecule is a macromolecular carrier, preferably HSA or avidin. In such instances, the macromolecular carrier can be bonded directly or indirectly to the metal ion complex, and the macromolecular carrier may further be loaded with members of a binding pair to facilitate targeting of the complex. Examples of these targeting complexes comprising a macromolecular carrier to which both the metal ion complex-deliverable compound complex and the members of a specific binding pair are bonded will exemplified in the description below. Other embodiments, wherein the macromolecular carrier comprises additional functionality-providing compounds such as for instance PEG, will become clear from the description and examples. In another preferred embodiment, the targeting moiety comprises a low molecular weight protein such as for instance lysozyme or any other protein.

The deliverable compound may in preferred embodiments of the cell-targeting complex of the invention, comprise a protein, more preferably a recombinant protein. In order to facilitate bonding to the metal ion complex, the (recombinant) protein preferably comprises at least one residue selected from histidine, methionine and cysteine, wherein the methionine and cysteine residue serves as a sulphur-containing reactive site and wherein a histidine residue serves as a nitrogen-containing reactive site. Highly preferred are proteins having histidine or methionine residues.

Alternatively, the deliverable compound may comprise or be a small molecule, a peptide, a (si)RNA, an anti sense (oligo) nucleotide, or modifications and combinations thereof.

In preferred embodiments of low molecular weight protein, said protein accumulates in the kidney of a mammal, including a human. A particularly preferred embodiment is lysozyme.

In still other preferred embodiments, the deliverable compound inhibits a signal transduction cascade in a cellular system, or has anti-inflammatory activity, anti-hypertensive activity, antifibrotic activity, anti-angiogenic activity, antitumor activity or apoptosis-inducing activity.

In still other preferred embodiments, the deliverable compound is an anti-inflammatory compound, preferably Pentoxifylline or PDTC.

In still other preferred embodiments, the deliverable compound is an anti-hypertensive agent, preferably Losartan.

In still other preferred embodiments, the deliverable compound is a kinase inhibitor, preferably PTKI, SB202190, PTK787, TKI, Y27632 or AG1295.

In still other preferred embodiments, the deliverable protein is a therapeutic protein, more preferably a cytokine or a growth factor. Alternatively, the therapeutic protein may be TNF-related apoptosis-inducing ligand (TRAIL). In still another alternative, the protein may be IL-10, alkaline phosphatase, superoxide dismutase, immunoglobulins or parts thereof (e.g. single chain, Fab-fragments or whole IgG), lactoferrin, xanthine oxidase, or TNFα.

In a further aspect, the present invention provides a pharmaceutical composition comprising a cell-targeting complex as described herein, and a pharmaceutically acceptable formulation for the intended application.

In yet another aspect, the present invention provides a cell-targeting complex as described above, for use as a medicament.

In a further aspect, the present invention provides the use of a (transition) metal ion complex capable of forming coordination bonds, for linking a targeting moiety to a deliverable compound. In preferred embodiments of this aspect, the metal ion complex is a platinum complex. Again, the platinum complex may be a trans-platinum complex or it may be a cis-platinum complex. In the case of a platinum complex, comprising a stabilising bridge, these platinum complexes preferably comprise an inert bidentate or tridentate moiety as a stabilising bridge. Suitably, the targeting moiety and therapeutic compound may comprise one or more sulphur-containing reactive sites and/or one or more nitrogen containing reactive-sites for participating in the coordination bond. Metal ion complexes of the present invention contain at least one (transition) metal ion. In another preferred embodiment the metal ion complex comprises two or more transition metals for binding a targeting moiety to a therapeutic compound. This embodiment is of course also a part of the targeting complex aspect described above.

The invention provides for the referred use wherein said deliverable compound is a diagnostic compound or therapeutic compound, preferably a therapeutic compound as the (transition) metal ion complex provides release functionality.

Particularly useful are therapeutic compounds that inhibit a signal transduction cascade in a cellular system, or that have anti-inflammatory, anti-hypertensive, antifibrotic, anti-angiogenic, antitumor or apoptosis-inducing activity.

Preferred compounds are, as stated earlier herein, kinase inhibitors or apoptosis inducing compounds.

Other preferred embodiments of the use according to the invention relates to embodiments wherein the therapeutic compound is pentoxifylline, PDTC, Losartan, PTKI, SB202190, PTK787, TKI, Y27632, AG1295 or TRAIL Highly preferred targeting moieties for application in the use of the present invention are mannose-6-phosphate modified human serum albumin or RGD-loaded albumin, PEGylated albumin, a low molecular weight protein, an Ab or an Ab fragment or a polymer or a modified polymer.

In a further aspect, the present invention provides a method of coupling a targeting moiety to a therapeutic compound comprising a (transition) metal ion complex having at least a first reactive moiety for forming a coordination bond with a reactive site of said targeting moiety and having at least a second reactive moiety for forming a coordination bond with a reactive site of said therapeutic compound and allowing said (transition) metal ion complex to form coordination bonds with said targeting moiety and said therapeutic compound. Preferred embodiments of this aspect of the invention include those embodiments as described for the cell-targeting complex and the use of the invention as described above.

In a further aspect, the present invention provides a method of targeting therapeutic compounds to selected cell populations, comprising administering to a subject in need thereof a therapeutically effective amount of a pharmaceutical composition according to the present invention.

### DESCRIPTION OF THE FIGURES

FIG. 1 shows the reaction scheme of the modified [Pt(ethylenediamine)dichloride] systems trans-[Pt(NH3)2(ptx)(NH2(CH)nNH2)] (n=number of C atoms in the aliphatic chain of the linkers).

FIG. 2 shows the reaction scheme of the modification of the NH2 group of the linker.

FIG. 3 shows the synthetic scheme for the preparation of the dinuclear conjugate 4.

FIG. 4 shows drug release profiles of cell targeting complexes. A: incubation of PTX-cisULS-M6PHSA in serum/PBS 1:1; B: incubation of PTX-cisULS-M6PHSA in different media for 24 h; C: SB202190-cisULS-LZM. See Example 2.1 for details.

FIG. 5. Incubation of HSC with PTX-cisULS-M6PHSA for 24 h, followed by immunohistochemical staining for fibrosis markers. Panel A,B) controls; panel C,D) PTX-cisULS-HSA; panel E,F) PTX-cisULS-M6PHSA; panel G,H) non-targeted PTX. Activated HSC express collagen type I in a granular staining pattern in the cytoplasm, probably reflecting the presence of procollagen type I, while αSMA stained in a fibre-like pattern. Treatment with PTX in a concentration of 1 mM reduced the red coloured intensity of both fibrotic markers. Incubation of the cells with PTX-cisULS-M6PHSA affected collagen type I expression considerably (E). Although the αSMA-staining intensity was not affected by PTX-cisULS-M6PHSA, pronounced changes in the morphology of HSC were observed (F). These effects were not observed after incubation with equivalent concentrations of PTX-HSA (C,D). See Example 2.2 for details.

FIG. 6. Effects of PTKE-cisULS-M6PHSA on gene-expression in HSC (panel A) or liver slices from fibrotic rat livers (panel B). See Example 2.2 for details.

FIG. 7. Effect of RGD-PEG-SB202190-cisULS-HSA on gene-expression in endothelial cells. TNF-activated HUVEC were incubated with indicated compounds. Panel A: gene-expression analysis; panel B: determination of IL-8 in culture medium. See Example 2.2 for details.

FIG. 8. Effects of SB-cisULS-LZM (panel A) and TKI-cisULS-LZM (panel B) on the gene expressions of procollagen-Ia1 induced by TGF-β1 in HK-2 renal tubular cells. See Example 2.2 for details.

FIG. 9. Effects of RGD-equipped TK787-albumin conjugates on gene-expression in endothelial cells. VEGF-activated HUVEC were incubated with indicated compounds. See Example 2.2 for details.

FIG. 10. Incubation of HSC with cell targeting complexes. A-D: PTX-cisULS-M6PHSA; A: cell viability; B: caspase 3/7 activity; C: TUNEL staining; D: quantification of TUNEL positive nuclei (TUNEL/DAPI ratio). E: losartan-cisULS-M6PHSA, cell viability assay; F: PTKI-cisULS-M6PHSA, cell viability assay. See Example 2.3 for details.

FIG. 11. Incubation of NRK-52E cells with cell targeting complexes and comparison of cellular toxicity with other compounds. A: incubation with cisplatin or cisULS; B: incubation with SB202190 or SB202190-cisULS; C: incubation with LZM or SB202190-cisULS-LZM. See Example 2.3 for details.

FIG. 12. Incubation of HUVEC with RGDPEG-SB202190-cisULS-HSA: incubation for 3 days followed by MTS cell viability assay. See Example 2.3 for details.

FIG. 13 Incubation of HUVEC with non-modified hisTRAIL and biotinylated TRAIL (panel A) or RGD-equipped TRAIL (panel B) for 48 h, followed by MTS cell viability assay. See Example 2.3 for details.

FIG. 14. Incubation of Jurkat T-cells with cell targeting complexes. A,C: cell viability assay after 48 h of incubation; B,D: caspase activity assay after 4 h incubation. See Example 2.3 for details.

FIG. 15. A: Binding of PTX-cisULS-M6PHSA to fibroblasts. A: cell-associated radioactivity after incubation with radiolabeled PTX-cisULS-M6PHSA with/without competitor M6PHSA; B: anti-HSA immunodetection of cellular binding. Cells incubated with PTX-cisULS-M6PHSA showed intensely red staining of cell-associated or internalized conjugate, not observed after incubation with PTX-cisULS-HSA. See Example 2.4 for details.

FIG. 16. Binding of cell targeting complexes to endothelial cells: SB202190-cisULS equipped complexes. Cell-associated radioactivity after incubation with radiolabeled RGDPEG-SB202190-cisULS-HSA. A: incubation at 4° C. for 4 h; B: incubation at different temperatures and different duration. See Example 2.4 for details.

FIG. 17. Binding of cell targeting complexes to endothelial cells. Binding affinity was determined by competitive displacement of radiolabeled echistatin. A-D: incubation with RGD-equipped PTK787-cisULS complexes; E: incubation with RGDPEG-avidin. See Example 2.4 for details.

FIG. 18. Pharmacokinetics of PTX-cisULS-M6PHSA in BDL3 rats. Organ distributions were determined in BDL rats at 10 min administration of the compounds. See Example 3.1.1 for details.

FIG. 19. A: Organ distribution of losartan-cisULS-M6PHSA in BDL rats. Anti-HSA staining of lung (a), spleen (b), heart (c), kidney (d), liver (e). anti-HSA and anti-desmin double staining in liver (f, g). Red color of HSA staining was not detected in lung, spleen, heart or kidney, while it was detected in the liver within the non-parenchymal cells of rats treated with losartan-cisULS-M6PHSA (magnification 4×). Losartan-M6PHSA co-localized with stellate cells in rat livers, as assessed with double immunostaining with anti-HSA and anti-desmin (arrows in f,g; magnification 40×). B: losartan tissue levels in the livers of CCL4 rats. See Example 3.1.1 for details.

FIG. 20. Pharmacokinetic evaluation of SB202190-cisULS-LZM. A,B: SB202190 levels in serum (A) or kidney (B). C: anti-LZM staining in kidney sections, arrows indicate accumulation of conjugate (red color) in tubular cells. See Example 3.1.2 for details.

FIG. 21. Pharmacokinetic evaluation of TKI-cisULS-LZM. A-C: TKI levels in serum (A) or kidney (B) or urine (C). D: anti-LZM staining in kidney sections, arrows indicate accumulation of conjugate (red color) in tubular cells. See Example 3.1.2 for details.

FIG. 22. Pharmacokinetic evaluation of Y27632-cisULS-LZM. See Example 3.1.2 for details.

FIG. 23. Pharmacological evaluation of losartan-cisULS-M6PHSA in BDL rats. A-D: Sirius Red staining for collagen in fibrotic liver. Severe bridging of collagen (red color) was observed in rats receiving saline (A), M6PHSA (B) and oral losartan (D). However, rats treated with losartan-M6PHSA (C) showed fewer areas with collagen accumulation. E: morphometric quantification of Sirius Red staining. F: gene-expression analysis of procollagen al. See Example 3.2.1 for details.

FIG. 24. Pharmacological evaluation of losartan-cisULS-M6PHSA in CCl₄ rats. A-D: Sirius Red staining for collagen in fibrotic liver. Red color indicates the deposition of collagen in the liver, which was markedly reduced after treatment with losartan-cisULS-M6PHSA but not after M6PHSA or free losartan. E: morphometric quantification of Sirius Red staining. See Example 3.2.1 for details.

FIG. 25. Pharmacological evaluation of losartan-cisULS-M6PHSA in fibrotic rats. A-D: staining for αSMA in fibrotic livers of BDL rats. Treatment with treatment losartan-cisULS-M6PHSA but not with M6PHSA or free losartan reduced the positively stained area (brown color). E: High magnification photomicrograph of a saline treated BDL rat. Arrows denote SMA in activated HSC-derived (upper) or myofibroblasts surrounding proliferating bile ducts (lower arrow). F: morphometric quantification of SMA staining in BDL rats. G,H: staining for αSMA in fibrotic livers of CCL4 rats. Treatment with losartan-cisULS-M6PHSA reduced the intensity of staining (reduction in brown color). I: morphometric quantification of SMA staining in CCL4 rats. See Example 3.2.1 for details.

FIG. 26. Pharmacological evaluation of losartan-cisULS-M6PHSA in BDL rats. A-D: CD43 staining for infiltrated immune cells in fibrotic liver. Rats receiving saline (A) or M6PHSA (B) showed intense infiltration by CD43 positive cells (brown color). Treatment with losartan-M6PHSA (C), an in a lesser extent, or oral losartan (D), induced a reduction in the number of CD43 infiltrating leukocytes. E: morphometric quantification of CD43 staining of the number of positive cells in 20 randomly chosen high power fields. See Example 3.2.1 for details.

FIG. 27. Pharmacological evaluation of PTKI-cisULS-M6PHSA in BDL rats. A: morphometric quantification of Sirius Red staining for in fibrotic liver. B: photomicrographs of Sirius red staining of livers 1 day after administration of compounds (BDL11) or 2 days after administration (BDL12). Treatment with PTKI-cisULS-M6PHSA reduced the brown-reddish color of stained collagen at both time points, and also reduced the bridging between collagen-rich areas. See Example 3.2.1 for details.

FIG. 28. Pharmacological evaluation of PTKI-cisULS-M6PHSA in BDL rats. A: morphometric quantification of αSMA staining for in fibrotic liver. B: photomicrographs of αSMA staining (brown color) of livers 1 day after administration of compounds (BDL11) or 2 days after administration (BDL12). Treatment with PTKI-cisULS-M6PHSA reduced the brown-reddish color of stained SMA at both time points. See Example 3.2.1 for details.

FIG. 29. Pharmacological evaluation of SB202190-cisULS-LZM in I/R rats. A-C: staining for phosphorylated p38 in control I/R rats (A), SB202190-cisULS-LZM treated I/R rats (B) or SB202190 treated I/R rats (C). D-F: staining for αSMA in control I/R rats (D), SB202190-cisULS-LZM treated I/R rats (E) or SB202190 treated I/R rats (F). Arrows in panels A-C indicate the immunolocalization (dark brown color) of p-p38 positive nuclei in the tubular cells corresponding to the dilated and injured tubules. Arrows in panels D-F indicate the localization of αSMA (red color) in tubulointerstitial space. Treatment with SB202190-cisULS-LZM reduced the number of p-p38 positive cells and the expression of αSMA as compared to I/R, controls, while free SB202190 did not affect either parameter. See Example 3: 2.2 for details.

FIG. 30. Pharmacological evaluation of losartan-cisULS-LZM in I/R rats. Infiltrated immune cells in the kidney were detected by ED1 staining and counted by two independent observers. See Example 3.2.2 for details.

FIG. 31. Pharmacological evaluation of Y27632-cisULS-LZM in I/R rats. Renal gene expression levels were determined by qRT-PCR and expressed relative to the levels of control I/R animals. See Example 3.2.2 for details.

FIG. 31A presents the gene expression levels in the kidney. In comparison to normal rats, vehicle-treated I/R, animals had a significant increase in the gene expression of the inflammation marker MCP-1 and of fibrosis markers α-SMA, TGF-β1, procollagen-Iα1 and TIMP-1. Data represents mean±SEM. ††p<0.01 and †††p<0.001 represent the difference versus normal rats. Other differences are indicated as *p<0.05 and ** p<0.01. Immunohistochemical evaluation of kidney sections demonstrated beneficial effects of Y27632-cisULS-LZM at the infiltration of macrophages (ED-1 staining, FIG. 31B) and at the expression of the fibrotic marker α-SMA (FIG. 31C). While both markers were absent in control kidney sections and extensively increased in I/R animals, treatment with Y27632-cisULS-LZM and to a lesser extent Y27632 significantly reduced the morphometric score of the stained areas.

FIG. 32. Pharmacological evaluation of Y27632-cisULS-LZM in UUO rats. A: Relative gene expression in the kidney of MCP-1; B: quantification of renally infiltrated macrophages; C: quantification of SMA staining in the kidney. See Example 3.2.2 for details.

FIG. 33 shows one possible configuration of a complex according to the present invention for the targeting of PTK787 as a deliverable compound. The Figure indicates that RGD, as an example of the homing part of the targeting moiety, may be coupled in various ways to the drug PTK787. PTK787 is coupled directly to the metal ion complex, whereas the cyclic RGD may be bonded to the metal ion complex via a carrier protein (HSA) or still further via an additional PEG-linker. The advantage of using HSA is that this protein is not foreign to human subjects, and thus does not exert an immune response.

FIG. 34 shows another possible configuration of a complex of the present invention for the targeting of a therapeutic compound (indicated is TRAIL). A, is the carrier/homing protein (for instance avidin, which has a high affinity for biotin and a high RGD loading capacity); B, is a versatile linker (for instance polyethylene glycol, serving as a bivalent linker for RGD conjugation, serving to prolong circulation time (avoiding non-target cell binding) and shielding the modified carrier (e.g from macrophage clearance)); C, is the homing molecule (for instance c(RGDf), which displays high affinity binding to α_{vβ}₃ integrin-expressing cells (endothelial cells in newly formed blood vessels)); D, is the deliverable compound (for instance the therapeutic protein human recombinant TRAIL, which induces selective apoptosis in tumor cells)); E, is a histidine tag suitable for purification purposes and coupling to the (transition) metal ion complex (e.g. the coordinated platinum compound); F, is a bio-linker (consisting of for instance a sulfo-NHS biotin part for direct coupling to a lysin residue, and a ULS biotin part, the platinum-based linker for drug conjugation)

### DETAILED DESCRIPTION OF THE INVENTION

### Definitions

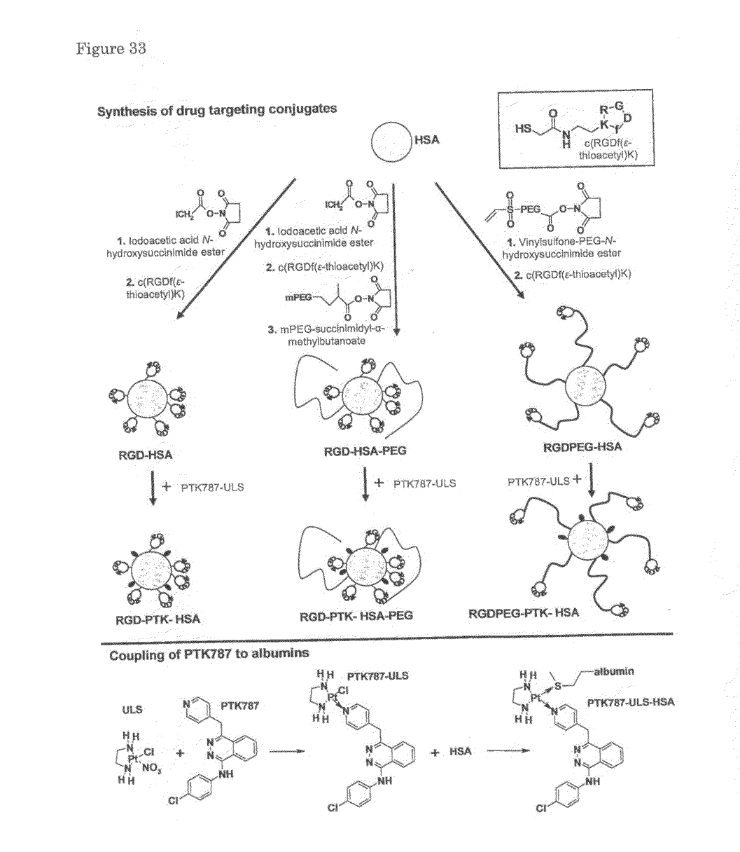

The term “targeting moiety” as used herein refers to any molecule or complex that can redirect, modify or improve the pharmacokinetic properties of a drug or other type of deliverable compound. In a preferred embodiment, the targeting moiety comprises a compound or part of a molecule which functionally interacts with a binding site on the surface of the cell, also indicated with the term “homing part”. Thus, the targeting moiety through the presence of homing part provides specificity for or binding affinity for one or more cell types. Alternatively, the targeting moiety may consist of a homing part. In such an embodiment, for instance an antibody, hapten or receptor ligand is bonded directly to the metal ion complex. The molecule on the cell which is targeted by the targeting moiety can be any suitable target, such as for instance a cell surface receptor. Targeting moieties may include such homing parts as, but are not limited to, antibodies, antibody fragments, endogenous or non-endogenous ligands for a cell-surface receptor, antigen-binding proteins, viral surface components, proteins, for instance those that bind viral surface components, growth factors, lectins, carbohydrates, fatty acids or other hydrophobic substituents, peptides and peptidomimetic molecules. Targeting moieties may either encompass complexes (see for instance RGD-HSA, RGD-HSA-PEG and RGDPEG-HSA in FIG. 33, wherein the homing part is complexed with macromolecules to yield multivalent structures) or may comprise or consist of a small molecule in the form of an antibody fragment or a small protein or peptide, such as for instance lysozyme.

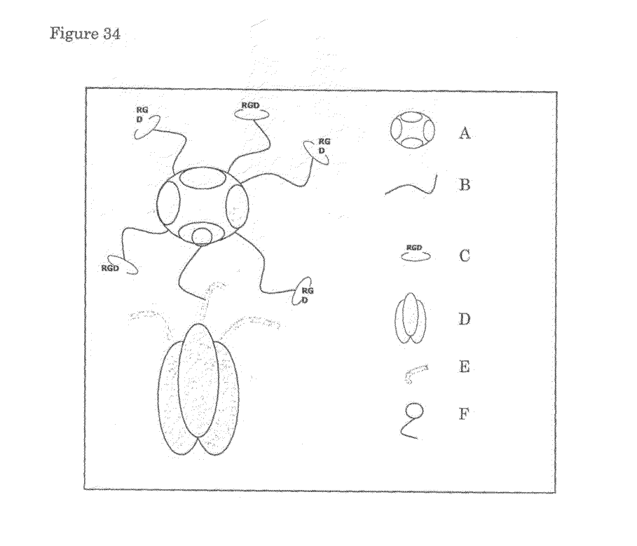

Very suitably, the targeting moiety may be a complex comprising a central macromolecular core as a carrier, to which one or more homing molecules are attached and optionally one or more functional groups which modify the functionality of the carrier. A very suitable example is that displayed in FIG. 34, wherein the macromolecule avidin is used to increase the capacity of the complex for loading with the specific homing molecule RGD. Optionally, the homing molecule may be coupled via a versatile linker, such as a PEG linker. Suitable macromolecular carrier compounds may thus for instance be avidin, human serum albumin (HSA) or dendrimers such as but not limited to PAMAM, or derivatives thereof. Preferred macromolecular carrier compounds in targeting moieties used in the present invention are Human Serum Albumin (HSA) or derivatives thereof, Since these are non-foreign to the human body. The term “derivatives thereof” in this context refers to macromolecular carrier compounds modified by attachment thereto of a homing molecule (e.g. RGD or M6P). Mannose 6 phosphate derivatised macromolecular carriers allow for the targeting of a deliverable compound to for instance the liver, in particular the stellate cell receptors of necrotized liver tissue).

HSA can suitably be used as a core protein, which may be substituted with polyethyleneglycol (PEG) groups, which infer stealth properties in the final construct. As such, these PEG ligands can prevent the uptake by non-target cell such as macrophages.

In another preferred embodiment, the targeting moiety does not bind to target receptors, but affects the body distribution by altering the clearance route or rate of the deliverable compound via other principles. This class of targeting moieties includes, but is not limited to, polyethylene glycol (PEG) polymers. Another non-limiting example of such a type of targeting moiety is a polymeric biomaterial which functions as depot that is releasing the coupled deliverable compound. Such a device may circulate in the blood stream or may be implanted in another part of the body, for instance subcutaneously. All above mentioned structures are collectively and individually called targeting moieties.

The term “deliverable compound” as used herein refers to a compound which is carried or transported by the targeting moiety to the surface and/or interior of a cell. In aspects of the present invention, the deliverable compound is a therapeutic or diagnostic compound. The terms “deliverable compound” and “therapeutic compound” and “drug” are used interchangeably herein and represent any agent that can be applied for therapeutic or diagnostic use. Also, the therapeutic compound may be a pro-drug, which requires (bio)chemical modification to reach its active form.

In a preferred embodiment, the therapeutic compound is an organic molecule that contains an aromatic nitrogen or a sulphur atom which can form a coordination bond with the linker. The therapeutic compound may also contain a prodrug moiety that contains an aromatic nitrogen or a sulphur atom which can form a coordination bond with the linker. In another preferred embodiment, the therapeutic compound is a therapeutically active peptide or protein. Furthermore, the therapeutic compound may be a small molecule, a peptide, a (si)RNA, an anti sense (oligo) nucleotide, or modifications and combinations thereof.

The term “reactive moiety” refers to a chemical group, a free orbital reactive site or a ligand of the metal ion complex or compound that is capable of forming a bond with a reactive site of either a targeting moiety or a deliverable compound.

The term “ligands” as used herein refers to a molecule that binds to another molecule, used especially to refer to a small molecule that binds specifically to a larger molecule, e.g., an antigen binding to an antibody, or ligand binding to a receptor, or a substrate or allosteric effector binding to an enzyme. The term “ligand” is also used to indicate molecules that donate or accept a pair of electrons to form a coordinate bond with the central metal atom of a coordination complex. Thus meaning of the term ligand is context-related.

The term “(transition) metal ion complex” as used herein refers to the linker system used to couple the targeting moiety to the deliverable compound. A characteristic of such complexes is the presence therein of coordination bonds. Metal ions suitable for use in a (transition) metal ion complex used in the present invention are metal ions capable of forming coordination bonds with ligands. Thus, transition metals such as Pt, Ru, Cu, Zn, Ni, Pd, Os and Cd are amenable for use in the present invention. The (transition) metal ion complex of the present invention consists of a central metal ion bound to a number of other molecules, termed ligands. The nature of the chemical bond formed between ligand and metal can be thought of as involving the donation of a pair of electrons present on the ligand molecule or, in molecular orbital terms, as a molecular orbital formed by combining a filled orbital of the ligand with a vacant orbital of the metal. Those atoms in the ligand molecule that are directly involved in forming a chemical bond to the metal ion are therefore termed the donor atoms, generally comprising elements of Groups V and W of the periodic table, with nitrogen, oxygen, sulfur, phosphorus and arsenic being those most commonly employed.

Molecules that contain two or more donor atoms capable of binding to a single metal ion are termed chelating agents, or chelators, and the corresponding metal complexes are called chelates. The number of donor atoms present in a given chelator is termed the denticity of that chelator, ligands possessing two donor sites being called bidentate, those with three donor sites, tridentate, and so forth. In general, the higher the denticity of a chelator the more stable are the chelates formed, up to the point at which the denticity of the chelator matches the maximum coordination number attainable by the particular metal ion of interest. The maximum coordination number of a given metal ion in a given oxidation state is an intrinsic property of that metal, reflecting the number of vacant orbitals and, hence, the number of chemical bonds it is able to form with ligand donor atoms. When all of the available vacant orbitals have been used to form bonds to donor atoms in the ligand or ligands, the metal is said to be coordinatively saturated.

Within the scope of the present invention, a chelator can be a targeting moiety, deliverable compound, or stabilising bridge. For example, pyrrolidine dithiocarbamate (PDTC) is a chelating deliverable compound.

For any given metal ion, useful ligands according to the present invention may be selected by one skilled in the art, employing the following criteria. Ligands must possess donor atoms (or sets of donor atoms in the case of chelating ligands) that favor binding to the target metal ion. The general preference of any given metal ion for particular donor atoms (generally selected from the group consisting of carboxylic, phenolic, or ether oxygen atoms, amine, imine, or aromatic nitrogen atoms and charged or neutral sulfur atoms) is well known in the art.

The ligands must also offer the prospect of forming ligand-metal linkages that are likely to display appropriate stability in vivo. That is, the ligand-metal linkage does not dissociate during the time required to transport the therapeutic compound to the target cell. In addition, but depending on the intended application for any given deliverable compound, the ligand-metal linkage must offer the prospect of drug-release inside the target cell or at the target site. For many metals of interest there exists considerable art relating to the in vivo stability of particular ligand-metal linkages, which may be used to guide ligand-metal selection.

Within the scope of the present invention, there are many useful ligands that have been shown to coordinately bond to metal ions; they may either be mono, bi, or tridentate, depending on the number of sites that the ligand binds to the metal ion.

The above described ligands may be bifunctional. A bifunctional ligand is a molecule that contains, in addition to at least one metal binding site (donor atom), a second reactive moiety through which the ligand may be linked to, for example, a protein, without significantly affecting the metal-binding properties of the ligand.

The present invention provides for a novel type of linker system or linker in which the deliverable compound is joined by means of one or more coordination bonds with the targeting moiety. Several important problems in the field of designing targeted drugs have motivated us to create such a novel transition metal based linker technology and the “cell targeting complexes” that result from combining such linkers with deliverable compounds and targeting moieties.

In this respect, many small organic drugs do not have functional groups that can be used for the chemical linkage technologies (e.g. ester linkage) that are commonly applied to couple drugs to a targeting moiety. Thus, the existing (covalent) linkage methods are insufficient to prepare a cell targeting complex with appropriate stability of the drug-carrier linkage in vivo as described above. In this respect, the linkage system is very important and should provide for bonding that prevents premature release of the drug from the carrier in the circulation, while not being too stable, so that the drug remains inactive even when accumulated at/or in the target site. The application of a novel linker technology as described in the present invention creates the opportunity to couple drug molecules via one or more coordination bonds with the (transition) metal complex. One major advantage of the described linker system is that it allows for the development of cell targeting complexes with drugs that could not be produced previously due to the lack of coupling technology.

Also, in the event of a coordinative bond between the linker and the deliverable compound, e.g. a drug, the bond may be reversed, especially when small electron rich materials are present, e.g. in vivo gluthathione, in vitro potassium thiocyanate. The structure of the deliverable compound is not affected or changed upon binding and release.

Also, natural occurring linker binding sites, e.g. nitrogen and sulphur, in the deliverable compound eliminates the need to modify chemically the deliverable compound.

Furthermore, the coupling of therapeutic proteins (such as anti-inflammatory cytokines) to targeting moieties often causes the formation of undefined complexes of variable size and composition. Partly, this problem originates from the chemistry applied in the conjugation of the targeting moiety to the therapeutic protein. Many covalent linkers are directed to reactive sites like terminal amino groups of lysyl residues that are abundantly present within the protein. A linker system that reacts at more specific locations in the protein, as defined by the distribution of cysteine, methione and/or histidine residues within the protein, will yield better defined cell targeting complexes. In a preferred embodiment, the cell targeting complex has been prepared by reacting a recombinant protein displaying a “methionine start codon” or a “histidine tag” sequence, or a combination of these sequences, that both can react with the transition metal linker.

A cell-targeting complex of the present invention may have various forms or constitutions wherein the targeting moiety and a therapeutic compound are joined by means of a (transition) metal ion containing linker system. In (transition) metal ion complexes, platinum is the preferred metal ion.

A cell-targeting complex of the invention may be represented by the formula:

DC-M-TM

wherein

DC is the deliverable compound,

TM is the targeting moiety, and

M is a (transition) metal ion linker system.

The deliverable compound and the targeting moiety may be coupled directly to the (transition) metal ion linker system by means of a coordination bond. Also, the deliverable compound or the targeting moiety may be coupled to an inert multiple dentate moiety or stabilising bridge in a metal ion complex. For instance, the deliverable compound or targeting moiety may be coupled to the ethylenediamine stabilising bridge of a cis-platinum complex. The (transition) metal atom then functions as a reactive moiety for coupling to the targeting moiety or deliverable compound only, and not (directly) to the deliverable compound or targeting moiety, respectively.

A cell-targeting complex of the present invention may comprise a spacer between the linker and the deliverable compound and/or targeting moiety. Such a cell-targeting complex may be represented by the formula:

DC—X₁-M-X₂-TM

wherein

DC, TM and M are as defined above, and

X₁ is an optional spacer group or prodrug coupled to the (transition) metal atom by means of a coordination bond, and wherein X₁ is coupled by other chemical bonding (e.g. by covalent bond) to the deliverable compound. X₁ may very suitably be a spacer comprising a (transition) metal or a (enzyme responsive) cleavage site to allow for release of the therapeutic compound from the metal ion complex, and

X₂ is an optional spacer group between the metal atom and the targeting moiety. X₂ may very suitably be a spacer comprising a (transition) metal or a (enzyme responsive) cleavage site to allow for release of the deliverable compound and metal ion complex from the targeting moiety.

In a preferred embodiment at least one of X₁ or X₂ is present.

In another preferred embodiment, both X₁ and X₂ are present.

Suitable types of spacer groups are molecules that allow for an appropriate distance between the metal ion complex and another functional group and also allow further modification of the linker complex. Preferred linkers are [NH₂—(CH₂)ₙ—NH₂] wherein n varies between 1 and 10, preferably between 1 and 6. For instance, the spacer diaminohexane[NH₂—(CH₂)₆—NH₂] has been proven very useful in preferred embodiments of this invention. Other preferred linkers are [NH₂—(CH₂)ₙ-A-(CH₂)ₙ—NH₂] wherein A is a heteroatom, preferable N or O, and n varying between 1 and 5. Yet, another preferred type of spacer is NH₂—(CH₂)ₙ—NHR wherein n can be any number between 1 and 6, and R may be independently chosen to be C(O)CH₂Br, CO—(CH₂)ₘ—(C₄H₂NO₂) wherein m is 1-10 preferentially 5, CO—(C₆H₁₀)—CH₂—(C₄H₂NO₂), or CO—(CH₂)₂—NH₂—CO—CH₂—Br. Another preferred type of spacer is NH₂—(CH₂)ₙ—NH—CO—(CH₂)ₙ-mPEG wherein n varies between 1 and 6 and mPEG represents polyethylene glycol whether or not having —(C₄H₂NO₂) bond thereto.

Also, the linker may be attached to—or being part of—the stabilizing bridge of the (transition) metal ion complex. Preferred structures are —(CH₂)ₙ—NHR wherein n can be any number varying from 1 to 10, preferentially 6, and R may be independently chosen to be C(O)CH₂Br, CO—(CH₂)ₘ—(C₄H₂NO₂) wherein m is 1-10 preferentially 5, CO—(C₆H₁₀)—CH₂—(C₄H₂NO₂), CO—(CH₂)₂—NH₂—CO—CH₂—Br, or —CO—(CH₂)ₙ-mPEG wherein n varies between 1 and 6 and mPEG represents polyethylene glycol whether or not having —(C₄H₂NO₂) bond thereto.

A cell-targeting complex of the present invention may comprise more then one (transition) metal ion linker system. A (transition) metal ion complex that contains two (transition) metal ions is referred to as a dinuclear species. A dinuclear species may be represented by the formula:

M₁-A-M₂

### Wherein:

M₁ and M₂ are (transition) metal ion linkers as defined above;

A is an aliphatic chain whether or not containing a heteroatom.

A therapeutic compound or targeting moiety linked to a platinum complex may be referred to as a Pt—S adduct (when attached to a sulphur containing reactive site), to a Pt—N adduct (when attached to a nitrogen containing reactive site), or in general to a Pt-adduct.

A sulphur containing reactive site may hereafter be referred to as a S-reactive site, and a nitrogen containing reactive site may hereafter be referred to as N-reactive site.

Methods using platinum complexes to label bio-organic molecules have been contemplated for a very long time. Platinum complexes may react with a variety of reactive moieties on biomolecules and various types of detectable marker moieties are known to have been adhered to ionic platinum.

The use of a cis-platinum complex for this purpose has for instance been described in WO96/35696, which discloses a method for linking bio-organic molecules and markers through cis-platinum complexes. In these complexes, two co-ordination sites are occupied by either ends of a stabilising bridge, such as an ethylene diamine group. Cis-platinum complexes are suitable for linking labels to several kinds of bio-organic molecules, such as peptides, polypeptides, proteins, and nucleic acids. Methods using trans-platinum complexes have also been reported (EP 0 870 770) as suitable for labeling a variety of bio-organic molecules and linking of labels thereto.

The present inventors have found that a particular advantage of the use of a (transition) metal ion complex as a linker between a deliverable compound and a targeting moiety in targeted drugs is that (transition) metal ion complexes provide for bonds that are strong enough to allow the deliverable compound to be transported to various tissues in vivo while remaining coupled to a targeting moiety. The reactivity of platinum complexes towards a variety of reactive sites in drug molecules is a benefit in the application as a drug linking system, since it allows straightforward conjugation reactions that result in quantitative yields of the desired products.

A further advantage of the use of (transition) metal ion complexes is that, depending on the reactive moieties used, such complexes may support the coupling of a wide variety of biologically active compounds to targeting molecules, whereby the chemistry of the targeting moieties as well as of the therapeutic compounds can vary greatly.

Another advantage is that targeting moieties or deliverable compounds may be linked via a suitable (transition) metal ion complex at histidine (His), methionine (Met) and/or cysteine (Cys) residues within protein or peptide chains. This provides for the possibility to link to different and multiple side groups in a protein or peptide chain, which may for instance allow for the linking of several deliverable compounds to one targeting moiety or vice versa. Furthermore, the linkage of drugs or targeting moiety groups via the metal ion complex to these amino acid residues will allow concomitant derivatization of lysine side chains by a conventional derivatization strategy, without interference of the two reactions. Thus, the coupling of either substituent to the core protein or peptide will not lower the reaction efficiency, as is observed in conventional strategies that aim at lysine residues with both types of reactions.

The specificity of the coupling reaction with (transition) metal ion complexes may be controlled such as to discriminate between coupling to sulphur containing reactive sites and nitrogen containing reactive sites in a targeting moiety or deliverable compound. Therefore, by using the (transition) metal ion linker according to the present invention one can direct the coupling of a targeting moiety or deliverable compound towards a specified reactive site within a targeting moiety or deliverable compound.

Platinum is a preferred metal ion in (transition) metal ion complexes.

Examples of preferred platinum complexes suitable for use in a method of the present invention are cis- or trans-platinum complexes of the formula [Pt(II)(X₃)(X₄)(T)(D)] or a cis-platinum complex of the formula [Pt(II)(X₅)(T)(D)].

Herein, Pt represents platinum (Pt), T and D represent the same or different reactive moieties, that participate in the reaction with a targeting moiety (T) or a deliverable compound (D), respectively. Another possibility is that the substituents at position T and D will be replaced by another ligand to form the complex with targeting moiety or a deliverable compound. The entities, X₃ and X₄ represent the same or different inert moieties, and X₅ represents an inert moiety that may act as a stabilising bridge such as a multiple dentate ligand, e.g. a bidentate ligand. A preferred group of bidentate ligands are aliphatic diamine bridges and diamine cyclo bridges, all sufficiently known by persons skilled in the art. An overview of preferred tridentates is given in patent application EP 04078328.4 which are incorporated herein by reference.

A structural representation of some examples of such platinum complexes is shown below:

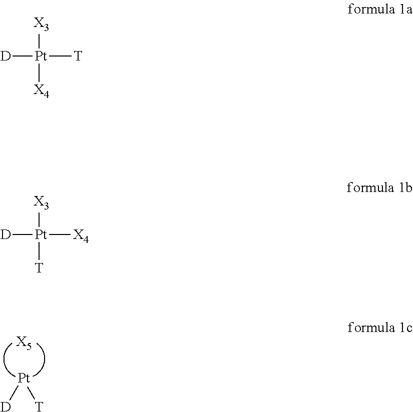

A platinum(II) complex, for use in a method of the invention can be prepared via any method known in the art. References can for example be found in Reedijk et al. (Structure and Bonding, 67, pp. 53-89, 1987). The preparation of some trans-platinum complexes is disclosed in EP 0 870 770. Further preparation methods can be found in WO 96/35696 and WO 98/15564. Methods described in any of these publications are incorporated herein by reference. In a preferred embodiment of the invention platinum complexes are prepared according to the spacer—tert butoxycarbonyl/NHS—pathway. In this approach, one of the ligands at the metal ion consists of a spacer molecule that contains an amino group distant from the metal ion that can be further derivatized for the purpose of conjugating either deliverable compound or targeting moiety.

Reactive moieties (T D) of a platinum complex that will be replaced by targeting moiety and deliverable compound are preferably good leaving ligands. A platinum complex, wherein T and/or D are independently chosen from the group of Cl⁻, NO₃⁻, HCO₃⁻, CO₃²⁻, SO₃²⁻, ZSO₃⁻, I⁻, Br⁻, F⁻, acetate, carboxylate, phosphate, ethylnitrate, oxalate, citrate, a phosphonate, ZO⁻, and water has been found to be particularly suitable for use in a method according to the invention. Z is defined herein as a hydrogen moiety or an alkyl or aryl group having from 1 to 10 carbon atoms. Of these ligands, Cl⁻ and NO₃⁻ are most preferred.

Any type of inert moiety X₅ may be chosen. Inert as used herein indicates that the moiety remains attached to the platinum complex during the linking process. A platinum complex comprising one or two inert moieties chosen from the group of NH₃, NH₂R, NHRR′, NRR′R″ groups, wherein R, R′ and R″ preferably represent an alkyl group having from 1 to 6 carbon atoms has been found to be particularly suitable for use in a method of the present invention. H₂NCH₃ is a particularly preferred inert moiety. An alkyl diamine, wherein the alkylgroup has 2 to 6 carbon atoms is a preferred bidentate inert moiety in a cis-platinum complex (e.g. X5 in formula 1c). In a particularly preferred embodiment X5 represents ethylene diamine.

Particularly preferred platinum complexes for use in a method of the present invention include cis[Pt(en)Cl₂], cis[Pt(en)Cl(NO₃)], cis[Pt(en)(NO₃)₂], trans[Pt(NH₃)₂Cl₂], trans[Pt(NH₃)₂Cl(NO₃)], and trans[Pt(NH₃)₂(NO₃)₂]; in these formulas, en indicates an ethylenediamine moiety.

In principle, any type of reactive site of a targeting moiety or deliverable compound may be used as coupling site in a method of the invention. Preferred nitrogen containing reactive sites include reactive sites comprising a primary amine, a secondary amine, a tertiary amine, an aromatic amine, an amide, an imide, an imine, an iminoether, or an azide. Preferred sulphur containing reactive sites include reactive sites comprising a thiol, a thioether, a sulfide, a disulfide, a thioamide, a thion, a dithiocarbamate. Examples of targeting moieties or deliverable compounds that can be coupled to the platinum-complex linker include organic drug molecules, proteins, amino acids peptides, oligopeptides, polypeptides, immunoglobulins, enzymes, synzymes, phospholipids, glycoproteins, nucleic acids, nucleosides, nucleotides, oligonucleotides, polynucleotides, peptide nucleic acids, peptide nucleic acid oligomers, peptide nucleic acid polymers, amines, aminoglycosides, nucleopeptides, glycopeptides and sugars, as well as combinations of the molecules listed above and any organic, inorganic or biological molecule that can be used to mimic these structures.

Proteinaceous deliverable compounds and targeting moieties may be facilitated with (enzyme responsive) cleavage sites by using recombinant protein technology. However, in a preferred embodiment the drug release facility is included in the linkage system. In another preferred embodiment the cleavage site is degradable by enzymes that are specifically present within the target area (metalloproteinases, plasmin or lysosomal proteases). In this way, additional site-specificity is created to cause release of pharmacologically active compounds only in or at the target site.

The deliverable compound may be any compound exerting therapeutic effect or having a diagnostic purpose. Non-limiting examples of deliverable compounds amenable for use in the present invention may be either proteinaceous macromolecules, or smaller chemical or nature-derived drug molecules. The therapeutic compound may for instance be a bioactive protein. Molecular biology has realised the possibility to design and produce recombinant proteins that can be used as a drug. Such proteins, like for instance cytokines and growth factors, are mediators with potent biological activities. However, therapeutic applications of cytokines are often limited because of the following considerations. Firstly, these potent mediators generally display multiple biological activities, that can also be harmful to the subject. The coupling of the deliverable to a targeting moiety improve the selectivity for an organ, tissue or cell type of the compound, and thereby greatly improve its therapeutic profile. Secondly, the plasma half-life of cytokines and many other recombinant proteins is generally short because of rapid elimination. Again, the coupling of a targeting moiety to the therapeutic compound may improve the pharmacokinetic properties of the compound. In addition, targeted delivery of recombinant proteins may allow the usage of lower doses, which can facilitate the successful application of therapeutic proteins that are produced in low yield. Successful drug targeting strategies may therefore open the application of a spectrum of therapeutic proteins.

Bio-active proteins and small drugs may in principle be coupled to a targeting moiety via a stable bond that is resistant to degradation, or by a bond that is cleaved within or outside the target cell resulting in the release of the pharmacological active compound from the carrier. The present invention allows for both types of bonds, for instance through modification of the co-ordination (platinum) compounds. The present invention relates to complexes wherein the targeting moiety is bonded to the deliverable compound via a (transition) metal ion complex having at least a first reactive moiety for forming a coordination bond with a reactive site of said targeting moiety and having at least a second reactive moiety for forming a coordination bond with a reactive site of said deliverable compound.

An important benefit of the present invention is now that the (transition) metal ion complex (for instance the ULS linker as presented herein) provides for a slow release of the deliverable compound from the targeting moiety. Thus, once homed-in on the desired organ, tissue or cell, the therapeutic compound is slowly released over a prolonged period of time, for instance multiple hours, days or weeks. As a result, the concentration of the free compound at any one time at a particular location can be controlled. Moreover, the therapeutic effect can be displayed for longer periods of time.

It is an additional advantage that the slow-release modality can be controlled by the chemistry of the coordination bond. The skilled person will understand that the presence of potential electron-donors in the environment of the coordination bond (for instance glutathione in the intracellular environment to which the complex of the invention is targeted) will result in release of the bond. Thus, the release can be environment-induced, based on the chemistry of the coordination bind, wherein a nitrogen-platinum bond is for instance stable but reversible, and forms a preferred embodiment.

Moreover, the therapeutic compound may often be a small molecule (drug) compound. It is a preferred embodiment in aspects of the present invention that the small molecule drug is attached directly via a coordination bond to the (transition) metal ion complex. This results in a reversible binding between the targeting complex and the therapeutic compound, and thus provides an inherent release function.

In one embodiment, the present invention provides for a targeting moiety in the form of a moiety that allows the targeting complex to be mobile (capable of moving through the body of the subject) until that point where the targeting moiety has recognized or has reached it's particular cellular target. In this case, the targeting moiety has the form of a selective homing moiety with specificity for a defined organ, tissue, cell, receptor or other biomolecule. For that purpose, the targeting moiety may comprise a binding pair or comprise a moiety with affinity for particular cell surface molecules such as receptors. Suitable are in this context the receptor ligands disclosed herein.

The same accounts for in situ diagnostic compounds, i.e. compounds which are used for imaging or detection of particular cell surface molecules on organs, tissues or cell types (tumours).

In another embodiment, the present invention provides for a targeting moiety in the form of a moiety that allows the targeting complex to be fixed to a particular location in the body, so that the slow-release modality of the complex may be confined to a specified location, in particular the location where the complex of the invention is applied. The complex may for that purpose comprise a targeting moiety containing a molecule that inhibits circulation in the body, for instance a PEG molecule. The skilled person will appreciate that other such molecules known in the art may also be used. The term “targeting” thus refers to the tendency of the complex to accumulate or remain at a particular site of the body of a subject.

A stable linkage between a protein and a targeting moiety may be realized by using a recombinant form of the protein that has been provided with a HIS-tag, i.e. a stretch of amino acid (histidine) residues attached to the protein molecule that is commonly used as an aid in its purification. The presence of both methionine residues and histidine residues in the HIS-tag make it amenable to react the metal ion linker to the HIS-tag, which may prove beneficial with respect to the therapeutic activity of the resulting complex. Furthermore, the metal ion linker may also be reacted with preference to methionine start codons of recombinant proteins that do not have a HIS-tag. This approach has wide commercial applicability because with this universal technology all recombinant protein drugs can be efficiently linked to targeting moieties by a method of the present invention.

Therapeutic compounds in the form of recombinant proteins may be produced by standard recombinant techniques. For instance, recombinant vectors encoding for the desired protein with a HIS-tag and optionally a cleavage site may be constructed by known methods.

A coupling method of the present invention comprises the step of allowing the (transition) metal ion complex to form coordination bonds with the targeting moiety and the deliverable compound.

Any order in which the bonds are formed is suitable. For instance, the (transition) metal ion complex may first form coordination bonds with the targeting moiety and, the resulting conjugate may subsequently be brought into contact with the deliverable compound so as to allow the formation of coordination bonds between the metal ion of the targeting moiety-metal conjugate and the deliverable compound. Also, all compounds may be brought together and the formation of the various bonds may commence simultaneously.

The bonds between the metal and deliverable compound and between the metal and the targeting moiety may be the same or different. For instance, the deliverable compound-metal bond may comprise a Pt—S adduct, while the targeting moiety-metal bond may comprise of a Pt—N adduct, or vice versa. Other types of bonds which will form coordination bonds with the metal ion may be selected by one skilled in the art.

The reaction parameters for the bonding reaction(s) may include the choice for a specific pH value. The pH as used herein should be interpreted as the pH value in water at 20° C. In general, the formation of Pt—S adducts is pH independent whereas formation of Pt—N adducts is pH dependent. In a preferred embodiment one or more S-reactive sites are selectively labelled over one or more nitrogen containing sites by making use of the pH.

As a guideline, one may choose the pH of the bonding reaction at a pH below the lowest pKa of any of a deliverable compound or targeting moiety N-reactive sites that should not be bonded, allowing preferential bonding to S-reactive sites. As the skilled professional will understand other factors, besides pKa, may play a role in the bonding reaction. In general, S-reactive sites are preferentially labelled over N-reactive sites at acidic pH. Therefore, the pH may be used for preferential binding of S based ligands over N based ligands.

In theory, the formation of a Pt—S adducts is a one step process. A reactive group leaves the metal ion complex upon S donating an electron pair to metal ion. This process, the direct conversion metal ion-X into metal ion-S, is believed to be pH independent. On the other hand, N donors require replacement of a reactive group of the metal ion complex by oxygen prior to N substitution. First, metal ion-X becomes metal ion-O and eventual metal ion-N. This is a two step scheme in which the first step can be controlled by changing pH. Factors influencing pH of a solution may therefore interfere with Pt—N adduct formation. The above described metal ion bonding relates in particular to Pt-ion coordination bonding.

The presence of ions may also be used to control the selectivity of the metal ion (preferably platinum) complex for forming coordination bonds with N-reactive sites. In an embodiment one or more leaving ligands, preferably anionic moieties, are used in the inhibition of the coupling of a metal ion complex to a N-reactive site, in order to enhance preferential coupling to an S-reactive site. Examples of such leaving ligands include Cl⁻, NO₃⁻, HCO₃⁻, CO₃²⁻, ZSO₃⁻, SO₃⁻, I⁻, Br⁻, F⁻, acetate, carboxylate, phosphate, ethylnitrate, oxalate, citrate, a phosphonate, ZO⁻, and water. Z is defined herein as a hydrogen moiety or an alkyl or aryl group having from 1 to 10 carbon atoms. Particularly good results have been achieved by using salts comprising an anionic moiety, of which chloride and nitrate is particularly preferred. The counter ions are preferably alkali cations, alkali earth cations or cations also used to direct the bond formation. In a preferred embodiment the total ionic strength of said anionic moieties used in the inhibition of bonding to a N-reactive site is at least 0.1 mol/l. More preferably the total ionic strength is in the range of 0.1 to 0.5 mol/l.

The presence of other (transition) metal ions in the reaction mixture, may also be used for selection of the reactive site to be labelled. In particular several (transition) metal ions have been found suitable to prevent or slow down bonding to an S-reactive site or to make a bonded Pt—S adduct labile, so that effectively N-reactive sites are preferentially bonded over S-reactive sites. Within a method according to the invention it is also possible to direct the bonding reaction by making use of geometrical isomers of a metal ion complex—e.g. a cis-platinum complex and a trans-platinum complex, —such that the metal ion complex is specifically bonded to either a sulphur containing reactive site or to a nitrogen containing reactive site.

This form of preferential bonding of targeting moieties or deliverable compounds may hold specific advantages when specific bonds between the various components of the complex of the invention are required and may thus be used to control the bonding of the ion metal complex to either the targeting moiety or the deliverable compound when the coupling reaction is performed in a reaction mixture comprising both the targeting moieties and deliverable compounds.

In addition to the parameters as mentioned above a method according to the invention may further be fine tuned by parameters such as temperature, preferably varied in the range between 0° C. and 120° C., more preferably in the range between 20° C. and 70° C.; reaction time, commonly in the range between 1 min and 48 hours, preferably in the range between 10 min and 24 hours, more preferably in the range between 25 min and 15 hours; concentration of the reagents, molar ratio of the reagents, overall net charge of the metal ion complex, and the like. These parameters may be adjusted depending upon the particular application in any way known in the art. The overall net charge of the metal ion complex, affects the specificity of metal ion-N adduct formation, for example, the specificity of Pt—N adduct formation in histidine at neutral pH. Neutral Pt-complexes form Pt—N adducts whereas positively charged platinum complexes do not. Positively charged Pt complexes display differential bonding towards N adducts above the isoelectric point of the peptide, protein, and the like. Apart from allowing the selective bonding to N-reactive sites over S-reactive sites or vice versa, a method according to the present invention also makes it possible to differentiate between distinct N-reactive sites or distinct S-reactive sites, by choosing the correct conditions, such as described in European patent application EP 1 262 778.

Purification, quality and stability of the conjugates may be performed and assessed using standard techniques. For instance, binding to and uptake of conjugates in target cells may be studied in-vitro using radiolabelled products or with immunohistochemical techniques. Suitable in-vitro test systems may for instance include cells and organ slices. Organ and cellular distribution of the developed complexes may for instance be studied in healthy animals and in experimental disease models (i.e. a rat or mouse models).

Once the cell-targeting complexes have been prepared, they may be combined with pharmaceutically acceptable carriers to form a pharmaceutical composition. As would be appreciated by one of skill in this art, the carriers may be chosen based on the route of administration as described below, the location of the target tissue, the drug being delivered, the time course of delivery of the drug, etc.

It is also an object of the invention to provide a method of targeting deliverable compounds to selected cell populations within the body. These and other objects can be addressed by providing a pharmaceutical composition comprising a cell-targeting complex according to the present invention. In a preferred embodiment the composition comprises a pharmaceutically acceptable carrier admixed with the cell-targeting complex of the invention. As used herein, the term “pharmaceutically acceptable carrier” means an inert solid, semi-solid or liquid filler, diluent, encapsulating material or formulation auxiliary of any type. A “pharmaceutically acceptable” carrier is one that is suitable for use with humans and/or animals without undue adverse side effects (such as toxicity, irritation, and allergic response) commensurate with a reasonable benefit/risk ratio. Such materials are pharmaceutically acceptable in that they are nontoxic, do not interfere with drug delivery, and are not for any other reasons biologically or otherwise undesirable. Remington's Pharmaceutical Sciences, 20th edition, Gennaro AR (ed), Mack Publishing Company, Easton, Pa., 2000 discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

A method of targeting deliverable compounds to selected cell populations comprises administering to a subject in need thereof a therapeutically effective amount of a pharmaceutical composition according to the invention. As used herein, “effective amount” means an amount of a drug or pharmacologically active compound that is nontoxic but sufficient to provide the desired local or systemic effect and performance at a reasonable benefit/risk ratio attending any medical treatment. As used herein, “administering” and similar terms mean delivering the composition to the individual being treated such that the composition is capable of reaching the intended target site or the systemic circulation. The composition is preferably administered to the individual by systemic administration, typically by intravenous, subcutaneous, intraperitoneal or intramuscular administration. Injectables for such use can be prepared in conventional forms, either as a liquid solution or suspension or in a solid form suitable for preparation as a solution or suspension in a liquid prior to injection, or as an emulsion. Suitable excipients or carriers include, for example, water, saline, dextrose, glycerol, ethanol, and the like; and if desired, minor amounts of auxiliary substances such as wetting or emulsifying agents, buffers, and the like can be added. Other carriers can be used and are well known in the art.

The pharmaceutical compositions of this invention can be administered to a subject by any means known in the art including oral and parenteral routes. The term “subject”, as used herein, refers to humans as well as non-humans, including, for example, mammals, birds, reptiles, amphibians and fish. Preferably, the non-humans are mammals (e.g., a rodent, a mouse, a rat, a rabbit, a monkey, a dog, a cat, a primate, or a pig). In certain embodiments parenteral routes are preferred since they avoid contact with the digestive enzymes that are found in the gastrointestinal tract.

The pharmaceutical compositions may be administered by injection (e.g., intravenous, subcutaneous or intramuscular, intraperitoneal injection), rectally, vaginally, topically (as by powders, creams, ointments, or drops), or by inhalation (as by sprays).

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the cell-targeting complex is suspended in carrier fluid comprising 1% (w/v) sodium carboxymethyl cellulose and 0.1% (v/v) TWEEN 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the cell-targeting complex with suitable non-irritating excipients or carriers such as cocoa butter, polyethylene glycol, or a suppository wax which are solid at ambient temperature but liquid at body temperature and therefore melt in the rectum or vaginal cavity and release the cell-targeting complex.

Dosage forms for topical or transdermal administration of an inventive pharmaceutical composition include ointments, pastes, creams, lotions, gels, powders, solutions, sprays, inhalants, or patches. The cell-targeting complex is admixed under sterile conditions with a pharmaceutically acceptable carrier and any needed preservatives or buffers as may be required. Ophthalmic formulations, ear drops and eye drops are also contemplated as being within the scope of this invention. The ointments, pastes, creams and gels may contain, in addition to the cell-targeting complex of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Transdermal patches have the added advantage of providing controlled delivery of a compound to the body.

Such dosage forms can be made by dissolving or dispensing the cell-targeting complex in a proper medium. Absorption enhancers can also be used to increase the flux of the compound across the skin. The rate can be controlled by either providing a rate controlling membrane or by dispersing the cell-targeting complex in a polymer matrix or gel.

Powders and sprays can contain, in addition to the cell-targeting complex of this invention, excipients such as lactose, talc, silicic acid, aluminum hydroxide, calcium silicates and polyamide powder, or mixtures of these drugs. Sprays can additionally contain customary propellants such as chlorofluorohydrocarbons.

When administered orally, the cell-targeting complex are preferably, but not necessarily, encapsulated. A variety of suitable encapsulation systems are known in the art (“Microcapsules and Nanoparticles in Medicine and Pharmacy,” Edited by Doubrow, M., CRC Press, Boca Raton, 1992; Mathiowitz and Langer J. Control. Release 5: 13, 1987; Mathiowitz et al., ReactivePolyp. ers 6: 275, 1987; Mathiowitz et al., J. Appl. Polymer Sci. 35: 755, 1988; LangerAcc. Chem. Res. 33: 94, 2000; Langer J. Control. Release 62: 7, 1999; Uhrich et al., Chem. Rev. 99: 3181, 1999; Zhou et al., J. Control. Release 75: 27, 2001; and Hanes et al., Pharm. Biotechnol. 6:389, 1995).

The cell-targeting complex may be encapsulated within biodegradable polymeric microspheres or liposomes. Examples of natural and synthetic polymers useful in the preparation of biodegradable microspheres include carbohydrates such as alginate, cellulose, polyhydroxyalkanoates, polyamides, polyphosphazenes, polypropylfumarates, polyethers, polyacetals, polycyanoacrylates, biodegradable polyurethanes, polycarbonates, polyanhydrides, polyhydroxyacids, poly(ortho esters) and other biodegradable polyesters. Examples of lipids useful in liposome production include phosphatidyl compounds, such as phosphatidylglycerol, phosphatidylcholine, phosphatidylserine, phosphatidylethanolamine, sphingolipids, cerebrosides and gangliosides.

Pharmaceutical compositions for oral administration can be liquid or solid.

Liquid dosage forms suitable for oral administration of inventive compositions include pharmaceutically acceptable emulsions, microemulsions, solutions, suspensions, syrups and elixirs. In addition to an encapsulated or unencapsulated cell-targeting complex, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions can also include adjuvants, wetting agents, emulsifying and suspending agents, sweetening, flavoring and perfuming agents. As used herein, the term “adjuvant” refers to any compound which is a nonspecific modulator of the immune response. In certain preferred embodiments, the adjuvant stimulates the immune response. Any adjuvant may be used in accordance with the present invention. A large number of adjuvant compounds is known in the art (Allison, Dev. Biol. Stand. 92: 3, 1998; Unkeless et al., Annu. Rev. Immunol. 6: 251, 1998; and Phillips et al., Vaccine 10: 151, 1992).

Solid dosage forms for oral administration include capsules, tablets, pills, powders and granules. In such solid dosage forms, the encapsulated or unencapsulated cell-targeting complex is mixed with at least one inert, pharmaceutically acceptable excipient or carrier such as sodium citrate or calcium phosphate and/or (a) fillers or extenders such as starches, lactose, sucrose, glucose, mannitol and silicic acid, (b) binders such as, for example, carboxymethylcellulose, alginates, gelatin, polyvinylpyrrolidinone, sucrose and acacia, (c) humectants such as glycerol, (d) disintegrating agents such as agar-agar, calcium carbonate, potato or tapioca starch, alginic acid, certain silicates and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well known in the pharmaceutical formulating art.

It will be appreciated that the exact dosage of the cell-targeting complex is chosen by the individual physician in view of the subject to be treated. In general, dosage and administration are adjusted to provide an effective amount of the cell-targeting complex to the subject being treated. As used herein, the “effective amount” of an cell-targeting complex refers to the amount necessary to elicit the desired biological response. As will be appreciated by those of ordinary skill in this art, the effective amount of cell-targeting complex may vary depending on such factors as the desired biological endpoint, the drug to be delivered, the target tissue, the route of administration, etc. For example, the effective amount of cell-targeting complex containing an anti-cancer drug might be the amount that results in a reduction in tumor size by a desired amount over a desired period of time. Additional factors which may be taken into account include the severity of the disease state; age, weight and gender of the subject being treated; diet, time and frequency of administration; drug combinations; reaction sensitivities; and tolerance/response to therapy. Long acting pharmaceutical compositions might be administered every 3 to 4 days, every week, or once every two weeks depending on half-life and clearance rate of the particular composition.

The cell-targeting complex of the invention is preferably formulated in dosage unit form for ease of administration and uniformity of dosage. The expression “dosage unit form” as used herein refers to a physically discrete unit of cell-targeting complex appropriate for the subject to be treated. It will be understood, however, that the total daily usage of the compositions of the present invention will be decided by the attending physician within the scope of sound medical judgment. For any cell-targeting complex, the therapeutically effective dose can be, estimated initially either in cell culture assays or in animal models, usually mice, rabbits, dogs, or pigs. The animal model is also used to achieve a desirable concentration range and route of administration. Such information can then be used to determine useful doses and routes for administration in humans.

Therapeutic efficacy and toxicity of cell-targeting complex can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies are used in formulating a range of dosage for human use.

The invention will now be illustrated by way of the following, non-limiting examples.

These examples illustrate the possibilities of the present invention to synthesise cell-targeting complexes with different classes of drugs and different classes of targeting moieties. Furthermore, they illustrate the possibilities of the present invention to developed constructs aimed at different target cells in different organs and diseases. Thus, it can be shown that the novel linker technology is not restricted to a single drug or class of drugs, nor to a single type of targeting moiety nor a single target cell type.

Rather, the cell-targeting complexes of the present invention may for instance target cells that play a major role in liver fibrosis (activated hepatic stellate cells), in renal fibrosis (kidney proximal tubular cells), in chronic inflammatory disorders (activated endothelial cells) or tumour angiogenesis (angiogenic endothelial cells). The drugs incorporated in the complexes may for instance intervene in inflammatory or fibrotic processes (pentoxyfillin, kinase inhibitors, angiotensin II receptor blockers, pyrrolidine dithiocarbamate), or programmed cell death (TNF related apoptosis inducing ligand). The potential usefulness of the synthesised cell targeting complexes will be demonstrated in drug targeting experiments with in vitro cultured target cells and in drug targeting studies in animal models.

### EXAMPLES

In the Examples below, use is made of the following therapeutic compounds.

| | |
|---|---|
| Pentoxifylline | 3,7-dimethyl-1-(5-oxohexyl)purine- |
| (TNF-alpha synthesis inhibitor; | 2,6-dione |
| anti-inflammatory agent) | |
| Losartan | 2-butyl-5-chloro-3-((4-(2-(2H-tetrazol- |
| (angiotensin II receptor | 5-yl)phenyl)phenyl)methyl)imidazol-4- |
| antagonist) | yl]methanol |
| PTKI | 4-Chloro-N-[4-methyl-3-(4-pyridin-3- |
| ((PDGF) receptor Tyrosine | yl-pyrimidin-2-ylamino)-phenyl]- |
| Kinase Inhibitor) | benzamide |
| SB202190 | 4-[5-(4-fluorophenyl)-2-(4- |
| (p38 MAP kinase inhibitor) | methylsulfinylphenyl)-3H-imidazol-4- |
| | yl]pyridine |
| PTK787 (ZK222584) | N-(4-chlorophenyl)-4-(pyridin-4- |
| (VEGF receptor Tyrosine | ylmethyl)phthalazin-1-amine |
| Kinase Inhibitor) | |
| TKI (ALK-5 inhibitor I) | 3-(Pyridin-2-yl)-4-(4-quinonyl)-1H- |
| (TGF-β RI Kinase Inhibitor) | pyrazole |
| Y27632 | 4-(1-aminoethyl)-N-pyridin-4-yl- |
| (Rho kinase inhibitor) | cyclohexane-1-carboxamide |
| | dihydrochloride |
| AG1295 | 6,7-Dimethyl-2-phenylquinoxaline |
| (PDGF-Receptor Tyrosine | |
| Kinase Inhibitor) | |
| PDTC | Pyrrolidine dithiocarbamate |
| (transcription factor nuclear | |
| factor kappa-B (NFjB) inhibitor; | |
| anti-inflammatory agents) | |

### Example 1

### Synthesis of Drug Delivery Complexes

### 1.1. Preparation of Drug-Linker Adducts and Drug-Linker-Carrier Complexes

A general protocol was developed for the synthesis of drug-carrier conjugates with the Pt(en(NO3)Cl) linker, which will be named cisULS linker hereafter. Typically, this type of cell targeting complexes was prepared by conjugation of the drug to the platinum containing linker, followed by reaction of the linker to a macromolecular carrier. In some of the examples, this approach yields a drug targeting preparation after the second reaction step. In another example, the complex was further modified with homing ligands to yield the final cell targeting complexes.

### General Conditions

Typically, platinum(ethylenediamine) dichloride (Pt(en)Cl2; 82.5 mg, 0.253 mmol) was dissolved in 3 ml of dimethylformamide (DMF) and converted into the more reactive Pt(en(NO3)Cl (cisULS mononitrate) by adding small aliquots of AgNO3 (38.5 mg, 0.227 mmol, dissolved in 1 ml of DMF) within a time period of two hours. The precipitated silver chloride was removed by centrifugation. Various drug molecules were reacted with the resulting cisULS mononitrate at the indicated conditions and molar ratios. Typically, reactions were conducted at a 1:1 molar until formation of the desired product was complete, as analyzed by HPLC. The resulting drug-ULS product was purified and reacted to various targeting devices or carriers, as illustrated below. Typical reaction conditions for the drug-ULS-carrier complexation are a 10:1 ratio of drug-ULS:carrier, a buffer of pH 8, a temperature of 37° C. and a reaction duration of 24 h. However, reaction conditions may vary depending on the composition of the product.

### 1.1.a. Synthesis of Pentoxifylline-cisULS Containing Complexes

### Synthesis of pentoxifylline-cis-ULS

Pentoxifylline (PTX, 43.2 mg, 0.155 mmol, dissolved in 1 ml of DMF) was reacted with cisULS (0.25 mmol) at 60° C. for 2 days. DMF was removed by rotary evaporation, after which the residue was dissolved in 2 ml of water and again taken to dryness under reduced pressure. The crude product was dissolved in water (2 ml) and stored overnight at −20° C., after which unreacted Pt(en)Cl2 precipitated as yellow crystals, which were removed by centrifugation (10 minutes at 14000 rpm). The identity and purity of the final product PTX-cisULS was checked by HPLC (95%), NMR and mass spectroscopy. PTX-cisULS was stored at 4° C. until further use.

Yield: 105 mg (0,166 mmol, 107% yield).

PTX-cisULS ¹H NMR (D₂O) δ_{H} 1.62 (m, 4H, CH₂(CH₂)₂CH₂), 2.25 (s, 3H, CH₃CO), 2.58 (m, 6H, COCH₂, H2N(CH₂)₂NH₂), 3.99 (t, J=6.56 Hz, 2H, CH₂N), 4.02 (s, 3H, CONCH₃), 4.55 (s, 3H, CNCH₃C), 5.59 (m, 4H, H₂N(CH₂)₂NH₂), 8.48 (s, 1H, CH) ppm.

PTX-cisULS ¹⁹⁵Pt NMR (D₂O): δPt −2471 ppm.

PTX-cisULS ionspray MS: calculated mass: 568. detected: 568 [M]; 550 [M-Cl+OH]. Both PTX-ULS species will react equally to the carrier in the second reaction step.

### Synthesis of pentoxifylline-cisULS-M6PHSA

In the present example, the anti-inflammatory compound PTX will be conjugated to the carrier protein mannose-6-phosphate modified human serum albumin (M6PHSA). M6PHSA binds to the insulin-like growth factor II/mannose-6-phosphate receptor, which is highly expressed during liver fibrosis on activated hepatic stellate cells (HSC)[1]. M6PHSA was synthesised as described previously [2] and stored after lyophilisation at −20° C. until further use. To conjugate PTX-cisULS to M6PHSA, 10 mg of M6PHSA (14.3 nmol) was dissolved in 1 ml of 20 mM tricine/NaNO3 buffer pH 8.3. PTX-cisULS (143 nmol) was added and the pH was checked and corrected if necessary. The mixture was reacted overnight at 37° C., after which unreacted PTX-cisULS was removed by size-exclusion chromatography using disposable PD10 columns (Amersham Biosciences), or by dialysis against PBS at 4° C. The final product was sterilised by filtration via a 0.2 μm filter and stored at −20° C.

PTX-cisULS-M6PHSA was characterised for protein content (BCA method) and for drug content by HPLC analysis. Typically, the coupled drug was released from M6PHSA by overnight incubation with 0.5M KSCN in PBS at 80° C. The released PTX was determined using a standard HPLC system equipped with a UV detector operated at 274 nm and a thermostated column oven operated at 40° C. Elutions were performed on a μBondapak Guard-pak C18 precolumn in combination with a 5 μm Hypersil BDS C8 column (250×4.6 mm, Thermoquest Runcorn, UK) using a mobile phase consisting of acetonitrile/water/trifluoracetic acid (25/75/0.1) at 1 ml/min. PTX eluted at 6 min after injection. Typically, the product contained 5-8 coupled drug molecules per M6PHSA when a 10-fold molar excess was used in the reaction.

### 1.1.b. Synthesis of Losartan-Containing Complexes

### Synthesis of losartan-cisULS

Losartan potassium (2-butyl-4-chloro-1-[p-(o-1H-tetrazol-5-yl-phenyl)benzyl]imidazole-5-methanol monopotassium) is an angiotensin II receptor (type AT₁) antagonist.

Losartan (32 μmol, 10 mg/ml of the potassium salt of losartan in DMF) was reacted with cisULS (32 μmol) at 60° C. for 3 days. Consumption of the starting material was monitored by analytical HPLC. Mass spectrometry analysis confirmed the presence of the 1:1 losartan-cisULS species after completion of the reaction.

¹H NMR of Losartan-cisULS (CD₃OD): δ_{H} 0.79 (m, 3H, CH₃), 1.25 (m, 2H, CH₂CH₃), 1.48 (m, 2H, CH₂CH₂CH₃), 2.50 (m, 6H, CH₂CH₂C and CH₂NH₂), 4.43 (m, 1.8H, NCH₂C), 5.18 (m, 2.2H, CH₂OH and remaining NCH₂C), 5.42 (m, 4H, NH₂); cyclic Hs: 6.82 (m, 0.2H), 6.87 (m, 1.8H), 7.04 (t, J=8.06 Hz, 1H, CHCHCH), 7.18 (m, 0.5H), 7.28 (m, 1H), 7.38 (m, 3H), 7.88 (m, 0.5H). ¹⁹⁵Pt NMR of Losartan-cisULS (CD₃OD): δ_{Pt}-2491 and −2658 ppm. MS (ESI⁺) m/z: 695 [M-K⁺-OH⁻-H]⁺, 677 [M-K⁺-Cl⁻-H⁺]⁺, 659 [M-K⁺-Cl⁻-H⁺-Cl⁻+OH⁻]⁺.

### Synthesis of losartan-cisULS-M6PHSA

In the present example, the angiotensin II receptor antagonist losartan will be conjugated to the carrier protein M6PHSA. As described above, this carrier binds to activated hepatic stellate cells (HSC) within the fibrotic liver. The coupled drug losartan interferes in the renin-angiotensin system. In brief, 10 mg of M6PHSA (14.3 nmol) was dissolved in 1 ml of 20 mM tricine/NaNO3 buffer pH 8.3. Losartan-cisULS (143 nmol) was added in a 10-fold molar excess and the pH was checked and corrected if necessary. The mixture was reacted overnight at 37° C., after which the product was purified by dialysis against PBS at 4° C. The final product was sterilised by filtration via a 0.2 μm filter and stored at −20° C. Losartan-cisULS-M6PHSA contained approximately 7 coupled losartan molecules per mole of protein, as was calculated form its protein content and drug content (HPLC). HPLC analysis of losartan was carried out using a Novapak C18 column and a mobile phase consisting of acetonitrile/water/trifluoroacetic acid at a 23/77/0.1 ratio. Eluted compounds were monitored at 225 nm.

### Synthesis of losartan-cisULS-Lysozyme

In the present example, the angiotensin II antagonist losartan will be conjugated to the carrier protein lysozyme (LZM). Since LZM and other low molecular weight proteins undergo glomerular filtration and subsequential uptake in proximal tubular cells, the resulting product losartan-cisULS-LZM will be accumulated in the kidney after in vivo administration. As such, the developed conjugate can serve in the delivery of the angiotensin antagonist to the kidney. One additional methionine group was introduced into LZM prior to the reacting with the drug-cisULS molecule. In brief, Boc-L-methionine hydroxysuccinimide ester (17 μmol; 10 mg/ml in DMSO) was added to lysozyme (14 μmol; 10 mg/ml in 0.1M sodium bicarbonate buffer pH 8.5) and stirred for 1 h at room temperature. The product was dialysed against water and lyophilised. The methionine-modified lysozyme contained one to two extra methionine groups as was confirmed by electron-spray mass analysis. The product was stored at −20° C. until further use. Losartan-cisULS (0.72 μmol; 3 mg/ml in DMF) was added to a solution of methionine modified LZM (0.36 μmol; 10 mg/ml in 20 mM tricine/sodium nitrate buffer pH 8.5) and the mixture was incubated at 37° C. for 24 h. The resulting product was purified by dialysis against water for 2 days, filtered through 0.2-μm membrane filter, lyophilised and stored at −20° C. The coupling of losartan with lysozyme in a 1:1 molar ratio was confirmed by mass analysis of the final product and HPLC analysis of the coupled drug after its release from the carrier.

### 1.1.c. Synthesis of PTSI-Containing Complexes

In the present example, a Platelet derived growth factor (PDGF) receptor Tyrosine Kinase Inhibitor (PTKI) will be conjugated to the carrier protein M6PHSA. As described above, this carrier binds to activated hepatic stellate cells (HSC) within the fibrotic liver. The coupled drug PTKI interferes in the PDGF kinase pathway. PDGF BB is one of the most potent mitogens for HSC proliferation and plays an important role in fibrotic processes.

### Synthesis of PTKI-cisULS

PTKI (4-Chloro-N-[4-methyl-3-(4-pyridin-3-yl-pyrimidin-2-ylamino)-phenyl]-benzamide, (7.2 μmol, 10 mg/ml in DMF) was mixed with an equimolar amount of cisULS (7.2 μmol, 20 mM in water). The reaction mixture was heated at 37° C. for 24 h after which consumption of the starting material was monitored by analytical HPLC. An additional amount of cis-ULS was added (0.5 equivalent, 3.6 μmol) and the reaction was continued for 48 h at 37° C. The reaction mixture was concentrated under reduced pressure and redissolved in methanol (6000). The crude product was purified by preparative HPLC and the collected peaks of the main product were taken to dryness under reduced pressure. The resulting white solid was treated with water to remove inorganic salts and dried. Yield: 0.9 mg (20%). Mass spectrometry analysis confirmed the presence of the 1:1 PTKI-ULS species. ¹H NMR analysis gave indication that binding of the inhibitor to Pt(II) takes place via the pyridyl nitrogen.

¹H NMR of PTKI (CD₃OD): δ_{H} 2.33 (s, 3H, CH₃), 7.26 (d, J=8.28 Hz, 1H, CCH₃CH), 7.37 (m, 2H, CHCl), 7.52 (m, 3H, N(CH)₂CCCH), 7.93 (d, J=8.60 Hz, 2H, CHCHCl), 8.22 (s, 1H, NHCCHC), 8.47 (d, J=5.23 Hz, 1H, CHNCNH), 8.64 (m, 2H, CH(CH)₂C and CHCHCNH), 9.29 (s, 1H, NCHC) ppm.

¹H NMR of PTKI-ULS (CD₃OD): δ_{H} 2.27 (m, 3H, CH₃), 2.66 (m, 2H, CH₂), 2.74 (m, 2H, CH₂), 7.15 (m, 3H, CHCCH₃ and CHCl), 7.47 (m, 3H, N(CH)₂CCCH), 7.84 (d, J=5.23 Hz, 1H, CHCHCNH), 7.89 (m, 2H, CHCHCl), 8.39 (d, J=3.95 Hz, 1H, CHNCNH) ppm.

Mass spectrometry of PTKI-ULS (ESI+): calculated mass: 706 (m/z); detected masses: 706 [M⁺], 688 [M-Cl⁻+OH⁻]⁺, 670 [M-Cl⁻-H⁺]⁺, 669 [M-Cl⁻-2H⁺]⁺.

### Synthesis of PTKI-cisULS-M6PHSA

PTKI-ULS was conjugated to M6PHSA according to a similar protocol as has been described above for other drug-ULS-M6PHSA conjugates. PTKI-ULS-M6PHSA and M6PHSA were analyzed by size-exclusion chromatography and anion exchange chromatography to verify that coupling of PTKI-ULS did not alter the properties of the M6PHSA protein. The amount of PTKI coupled to M6PHSA was analyzed by isocratic HPLC after competitive displacement of the drug by overnight incubation at 80° C. with excess of the platinum ligand potassium thiocyanate (KSCN, 0.5M in PBS). Elutions were performed on a Waters system (Waters, Milford, Mass., USA) equipped with a 5 μm Hypersil BDS C8 column (250×4.6 mm, Thermoquest Runcorn, UK), a thermostated column oven operated at 40° C. and an UV detector operated at 269 nm. The mobile phase consisted of acetonitrile/water/trifluoracetic acid (40/60/0.1, pH 2) at a flow rate of 1.0 ml/min with a sensitivity of 0.01. Retention times: PTKI: 7 min; PTKI-ULS: 5 min.

### 1.1.d. Synthesis of SB202190-containing complexes

### Synthesis of SB202190-cisULS

SB202190 (4-(-Fluorophenyl)-2-(4-hydroxy-phenyl)-5-(4-pyridyl)-1H-imidazole, 5.43 μmol) was dissolved in DMF at a concentration of 10 mg/ml, and reacted with cisULS 5.43 μmol, 20.5 mM in DMF) at 50° C. for 2 h. The reaction was followed by HPLC. After the reaction was completed, the mixture was evaporated to dryness under reduced pressure, affording a pale yellow solid that was analysed by HPLC, ¹HNMR and electronspray mass spectrometry. Mass Spectrometry analysis confirmed the presence of the target 1:1 drug:ULS species and ¹H NMR studies indicated that binding of SB202190 to cis-ULS takes place via co-ordination of the N— donor of the pyridine ring contained in the drug to the Pt(II) metal.

Yield was 92%.

¹H NMR of free SB202190 (CD₃OD): δ_{H} 6.88 (d, J=8.74 Hz, 2H, F(CHCH)₂), 7.17 (m, 2H, N(CHCH)₂), 7.50 (m, 4H, (CHCH)₂OH), 7.82 (d, J=8.68 Hz, 2H, F(CHCH)₂), 8.41 (m, 2H, N(CHCH)₂) ppm.

¹H NMR of SB202190-cisULS (CD₃OD): δ_{H} 2.59 (m, 4H, H₂N(CH₂)₂NH₂), 5.58 (s, 2H, NH₂), 5.91 (s, 2H, NH₂), 6.89 (d, J=8.75 Hz, 2H, F(CHCH)₂), 7.22 (m, 2H, N(CHCH)₂), 7.53 (m, 4H, (CHCH)₂OH), 7.82 (d, J=8.73 Hz, 2H, F(CHCH)₂), 8.52 (m, 2H, N(CHCH)₂) ppm.

MS (ESI⁺) m/z: 622 [M+H]⁺, 585 [M-Cl⁻-H⁺]⁺.

### Synthesis of SB202190-cisULS-RGDPEG-HSA

In the present example, the p38 Mitogen Activated Pathway (MAP) kinase inhibitor SB202190 will be conjugated to the carrier protein human serum albumin (HSA). The resulting product will be equipped with RGD-peptide homing ligands that bind to αᵥβ₃ integrin, a receptor that is expressed by endothelial cells in newly formed blood vessels [3]. Furthermore, the HSA core protein will be equipped with polyethylene glycol (PEG) groups, which infer stealth properties in the final construct. As such, these PEG ligands can prevent the uptake by non-target cells. Other representatives of this class of RGD-equipped homing devices will be exemplified below.

In the first reaction step, 10 mg of HSA (14.3 nmol) was dissolved in 1 ml of 20 mM tricine/NaNO3 buffer pH 8.5. SB202190-cisULS (143 nmol) was added and the pH was checked and corrected if necessary. The mixture was reacted for 24 h at 37° C., after which unreacted SB202190-cisULS was removed by dialysis against PBS at 4° C. The final product was sterilised by filtration via a 0.2 μm filter and stored at −20° C. SB202190-cisULS-HSA was characterised for protein content (BCA method) and for drug content (HPLC). Furthermore, conjugation of SB202190-cisULS to the protein was verified by taking UV-spectra, since the final product displayed a specific absorption at 334 nm which was due to the conjugated drug and not present in the parent HSA. For HPLC determination of SB202190, the coupled drug was released from HSA by overnight incubation with 0.5M KSCN in PBS at 80° C. The released SB202190 was determined using a standard HPLC system equipped with a C18 μBondapak column and a UV detector operated at 254 nm. Compounds were eluted with a mixture of water/acetonitrile/trifluoroacetic acid 80/20/0.1 at 1 ml/min. Typically, the product contained 5-8 coupled drug molecules per HSA when a 10-fold molar excess of SB202190-cisULS was used in the reaction. In the final reaction step, SB202190-cisULS-HSA was modified with PEG and RGD-peptide groups. SB202190-cisULS-HSA dissolved in PBS was treated with a 50-fold molar excess of VNS-PEG-NHS (Nektar, Ala., USA; 20 mg/ml in water), which was added dropwise. The mixture was protected from light with tin foil and incubated for 1 h at room temperature on a rollerbank. Meanwhile, the cyclic RGD peptide c(RGDfK-Ata) (Ansynth Service, Roosendaal, The Netherlands) was dissolved at 10 mg/ml in a 1:4 acetonitrile/water mixture. The RGDpeptide was added dropwise to the reaction mixture at a mole:mole ratio of 55:1, after which hydroxylamine was added in a final concentration of 50 mM. The reaction was carried out overnight at room temperature while protected from light. Remaining VNS groups were quenched by addition of cysteine (55:1 molar excess over the amount of HSA), after which the product was dialysed against PBS, and finally purified by size exclusion chromatography using a superdex200 column on an Äkta System (Amersham Pharmacia, Uppsala, Sweden). The final product was stored at −20° C. SB202190-cisULS-RGDPEG-HSA was characterised for its protein content and drug content as described above for SB202190-cisULS-HSA. Analytical size exclusion chromatography was performed to reveal increase in size, due to PEGylation, and to determine purity of the products. SDS-PAGE followed by western blotting and immunodetection with an in-house prepared anti-RGD-peptide antiserum was performed to demonstrate binding of the RGD peptide to the complex.

### Synthesis of SB202190-cisULS-Lysozyme

In the present example, SB202190 will be conjugated to the carrier protein lysozyme (LZM). The developed SB202190-cisULS-LZM conjugate can serve in the delivery of the p38 MAPkinase inhibitor to the kidney. Methionine-modified lysozyme (met-LZM, see above; 2.6 μmol; 10 mg/ml in tricine/sodium nitrate buffer pH 8.5) was treated with SB202190-ULS (7.8 μmol; 10 mg/ml in DMF), after which the mixture was incubated at 37° C. for 24 h. The product was dialysed against water, filtered through 0.2-μm membrane filter, lyophilised and stored at −20° C. The coupling of SB202190 with lysozyme in a 1:1 ratio was confirmed by electron-spray mass analysis of the final product. Furthermore, coupling of the drug was confirmed by HPLC analysis of the coupled SB202190 after release of the drug from the SB202190-lysozyme conjugate. For this purpose, the conjugate was incubated with 0.5M potassium thiocyanate (KSCN) dissolved in 0.1M phosphate buffer saline at 80° C. for 24 h to release SB202190 completely. SB202190 was estimated using HPLC (Waters, Milford, Mass., USA) analysis method described elsewhere. Briefly, the samples were added with an internal standard, SB202190 derivative, and extracted with 2 ml of diethylether three times. The extract was dried at 50° C. and the residue was reconstituted in 100 μl of mobile phase (acetonitrile:water:trifluoroacetic acid, 30:70:0.1 v/v/v). Twenty-five microliter of it was injected into HPLC column (Thermo-Hypersil Keystone, Bellefonte, Pa.) and detected at 350 nm. The peak height ratios of SB202190 and internal standard were used to calculate the concentration. The degree of SB202190 substitution in the conjugate was found to be 1 mole of SB202190 per mole of lysozyme. No free SB202190 was found in the final preparation.

### Synthesis of cisULS-dendrimer complexes

Dendrimers are branched polymers that can be equipped with drugs and homing devices in an analogous manner as described above for albumin-based drug delivery systems. The present examples describe the development of drug-dendrimer conjugates prepared with PAMAM dendrimers. The developed products can be applied for drug delivery to various targets, depending on the molecular size and further derivatizion with homing devices or other functional groups like PEG. This latter class of products is exemplified by the RGD_PEG equipped drug-dendrimer complex.

### Synthesis of SB202190-cisULS-dendrimers

PAMAM-NH2 dendrimer (generation 3) was equipped with methionine groups by reacting Boc-L-methionine hydroxysuccinimide ester (14.4 μmol; 10 mg/ml in acetonitrile) with the dendrimer (1.4 μmol, 10 mg/ml in PBS) for 1 h at room temperature. The product was purified by dialysis. against water and lyophilized. PAMAM-NH2 dendrimer (generation 4) was equipped with methionine groups (synthesis ratio 35:1) following the same protocol, and with p-isothiocyanate-benzyl-DTPA groups (synthesis ratio 3:1) to facilitate labeling of the complexes with a radioactive tracer isotope. The resulting carrier was purified by dialysis against PBS.

G3-Dendrimers were reacted with SB202190-cisULS (synthesis ratio 4:1) while G4-dendrimers were reacted at a 40:1 ratio, according to the protocol described above for other drug-carrier complexes. the final products were purified by dialysis and contained an average of 1:1 (G3D) or 20:1 (G4D) drug/dendrimer, as determined by UV absorbance at 360 nm (absorbance of conjugated SB201190-ULS).

### Synthesis of RGD-PEG equipped SB202190-cisULS-dendrimer

PAMAM-NH₂ dendrimer (generation 4) was equipped with VNS-PEG-NHS (synthesis ratio 40:1) and RGD-peptide (ratio 50:1) according to a similar protocol as described below for RGD-PEG equipped albumin carriers. Modification of the RGD-PEG-PAMAM dendrimer with 40:1 molar equivalents of SB202190-cisULS was carried out as described above for the PAMAM generation 4 dendrimer, and yielded a complex with an average of 15:1 drug/dendrimer, as determined by UV absorbance of the conjugated drug.

### 1.1.e. Synthesis of PTK787-containing complexes

In the present example, the vascular endothelial cell growth factor (VEGF) receptor tyrosine kinase inhibitor PTK787, which is a potent inhibitor of angiogenesis, will be conjugated to RGD-equipped human serum albumins.

### Synthesis of PTK787-cisULS

PTK787 (5.8 tμmol, 10 mg/ml in DMF) was mixed with cisULS (5.8 μmol). The resulting solution was heated at 37° C. for 24 hours during which consumption of the drug starting material was monitored by analytical HPLC. The solvents were removed under reduced pressure and taken-up 50:50 DMF:water. Mass Spectrometry analysis confirmed the presence of the target 1:1 drug:ULS species and ¹⁹⁵Pt NMR and ¹H NMR studies indicated that binding of PTK787 to cisULS takes place via co-ordination of the N— donor of the pyridine ring.

¹⁹⁵Pt NMR of [Pt(ethylenediamine)dichloride]: −2075 ppm.

¹⁹⁵Pt NMR of PTK787-ULS: −2493 ppm.

¹H NMR of PTK787 (CD₃OD): δ_{H} 4.64 (s, 2H, CH₂), 7.33 (m, 4H, CH₂C(CH)₂ and CHCH(CC)₂), 7.82 (d, J=8.63 Hz, 2H, NHC(CH)₂), 7.89 (m, 2H, (CH)₂CCl), 8.04 (d, J=7.80 Hz, 1H, CHCHCCNH), 8.40 (d, J=6.06 Hz, 2H, CHN), 8.44 (d, J=7.74 Hz, 1H, CHCHCCCH₂) ppm.

¹H NMR of PTK787-cisULS (CD₃OD): δ_{H} 2.57 (m, 2H, CH₂NH₂l), 2.65 (m, 2H, CH₂NH₂), 7.36 (d, J=8.84 Hz, 2H, CHCH(CC)₂), 7.48 (d, J=6.51 Hz, 2H, CH₂C(CH)₂), 7.82 (d, J=8.48 Hz, 2H, NHC(CH)₂), 7.95 (t, J=8.61 Hz, 2H, (CH)₂CCl), 8.10 (d, J=7.25 Hz, 1H, CHCHCCNH), 8.48 (d, J=7.58 Hz, 1H, CHCHCCCH₂), 8.61 (d, J=6.65 Hz, 2H, CHN) ppm.

Mass spectrometry of PTK787-ULS (ESI⁺): Theoretical m/z: 637.45 [M].

Detected masses m/z: 658 [M+Na⁺-H⁺], 637 [M]⁺, 601 [M-Cl⁻-H⁺]⁺.

UV/Vis (in PBS): λₘₐₓ 339 nm (ε=11679 M⁻¹ cm⁻¹).

### Conjugation of PTK-ULS to RGD-Equipped Carriers

Three different types of carriers have been developed by derivatisation of albumin with PEG and RGD-peptides. The incorporation of PEG in these conjugates (HSA) improves the solubility and will prevent non-specific uptake by macrophages. PEG furthermore will alter the body distribution of the products, favouring long circulation behaviour. The appended RGD-peptide facilitates binding to angiogenic endothelium, as described above.

### Synthesis of RGD-HSA

HSA (30 mg, 444 nmol) dissolved in PBS was incubated with a 22-fold molar excess of iodoacetic acid N-hydroxysuccinimide ester (SIA-linker, SIGMA, MO, USA; 10 mg/ml in DMF, 9.7 μmol). Meanwhile, the RGD peptide c(RGDf(ε-S-acetylthioacetyl)K) (Ansynth Service, Roosendaal, The Netherlands) was dissolved at 10 mg/ml in a 1:4 acetonitrile/water mixture. The peptide (11.1 μmol) was added drop wise to the reaction mixture at a peptide to protein molar ratio of 25:1, after which hydroxylamine was added to a final concentration of 50 mM. Hydroxylamine will release the acetyl group of the RGD peptide to obtain a free sulfhydryl group. The reaction was carried out overnight at room temperature while protected from light after which the product was extensively dialyzed against PBS. The final product RGD-HSA was stored at −20° C. A control conjugate RAD-HSA was prepared according to the same protocol with the control peptide c(RADf(ε-S-acetylthioacetyl)K).

### Synthesis of RGD-HSA-PEG

RGD-HSA (13 mg, 193 nmol) dissolved in PBS was incubated with a 20-fold molar excess of mPEG succinimidyl α-methylbutanoate (mPEG-SMB, Nektar Therapeutics, USA; 20 mg/ml, 3.85 μmol) and incubated for 3 h at room temperature. The product was purified by size exclusion chromatography (SEC) using a Superdex200 HR 10/60 column on an Äkta System (GE Healthcare, Uppsala, Sweden). SEC was performed with 0.5 ml/min PBS and monitored at 214 nm, 280 nm and 339 nm. The final products RGD-HSA-PEG and RAD-HSA-PEG (prepared according to the same protocol) were stored at −20° C.

### Synthesis of RGDPEG-HSA

HSA (10 mg, 148 nmol) was dissolved in PBS and incubated with a 50-fold molar excess of vinylsulfone-polyethylene glycol-N-hydroxysuccinimide ester (VNS-PEG-NHS; Nektar, Ala., USA; 20 mg/ml in water, 7.4 μmmol). The mixture was protected from light with aluminum foil and incubated for 1 h at room temperature while gently shaking on a spiramix roller bank. RGD peptide (8.14 mop was added in a 55-fold molar excess over HSA followed by hydroxylamine addition as described above. Reaction was carried out over night at room temperature while protected from light. Remaining VNS groups were quenched by addition of cysteine (8.14 μmol; 55× molar excess over the amount of HSA), after which the product was purified by SEC as described above. The final products RGDPEG-HSA and RADPEG-HSA were stored at −20° C.

### Coupling of PTK787-ULS to RGD-Equipped Albumins.

RGD modified carriers were dissolved in PBS and subsequently incubated with 15-fold molar excess of PTK787-ULS for 24 h at 37° C., after which non-reacted PTK-ULS and aggregated product was removed by size-exclusion chromatography. The products were sterilized by filtration via a 0.2 μm filter and stored at −20° C.

### 1.1.f. Synthesis of TKI-Containing Complexes

In the present example, the ALK-5 inhibitor (TGF-beta Kinase Inhibitor, TKI) 3-(Pyridin-2-yl)-4-(4-quinonyl)-1H-pyrazole (CalBiochem) will be conjugated to the kidney-directed carrier protein LZM. TKI inhibits signalling cascades that are activated by TGF-beta, one of the most important profibrotic growth factors. It is also a potent inhibitor of p38 MAPkinase.

### Synthesis of TKI-cisULS

TKI (9.98 mg, 37 μmol) was dissolved in DMF at a concentration of 10 mg/ml after which an equimolar amount of a freshly prepared solution of cisULS was added. The mixture was heated at 37° C. overnight. The product was analyzed by HPLC, LC-MS and Pt-NMR These analyses confirmed a 1:1 coupling ratio of TKI and ULS. Yield: 77.3% (by HPLC).

### Synthesis of TKI-cisULS-Lysozyme

TKI-cisULS was reacted with met-LZM as described above for conjugation of other drug-ULS-carrier complexes. The product was purified by dialysis against water and lyophilized and stored at −20° C. Ion-spray MS analysis confirmed the formation of TKI-LZM conjugate. The amount of conjugated drug was quantified after releasing it from the linker. On average, 1-2 TKI molecules were conjugated per LZM.

### 1.1.g. Synthesis of Y27632-Containing Complexes

In the present example, the ROCK inhibitor Y27632 will be conjugated to the kidney-directed carrier protein LZM.

### Synthesis of Y27632-cisULS

Y27632 (38 μmol, 10 mg/ml in water) was basidified with 1M NaOH to pH 8.5 and reacted with cisULS (66 μmmol) at 50° C. overnight. Consumption of the starting drug and formation of the products was followed by HPLC and LC-MS. The excess of ULS is then precipitated by the addition an equivalent volume of a 1:1 DMF:water solution with a 20 mM concentration in NaCl and a subsequent overnight stirring at ambient temperature. Yellow solids then are removed by filtration. Yield: 50% (HPLC; for the crude product).

### Synthesis of Y27632-cisULS-Lysozyme

Y27632-cisULS was reacted with met-LZM as described above for conjugation of other drug-ULS-carrier complexes. The product was purified by dialysis against water and lyophilized and stored at −20° C. Ion-spray MS analysis confirmed the formation of the conjugate. The amount of conjugated drug was quantified after releasing it from the linker. On average, 1-2 Y27632 molecules were conjugated per LZM.

### 1.2. Synthesis of Drug-Trans Platinum Complexes

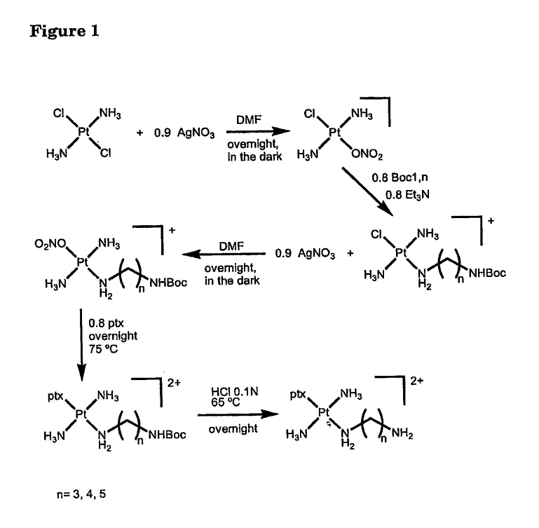

The synthesis of a number of drug-trans Platinum systems was performed in several steps, e.g. five steps as described below (see also FIG. 1).

### 1.—Trans-[PtCl(ONO₂)(NH₃)₂]

Transplatin (0.1 g, 0.333 mmol) in 4 ml of dimethylformamide (DMF) was treated with 0.9 equiv. of AgNO₃ (0.051 g, 0.3 mmol). The reaction mixture was stirred overnight, in the dark. After this time, AgCl was filtered off and the yellow solution, containing cis-[PtCl(ONO₂)(NH₃)₂], was checked by ¹⁹⁵Pt NMR. A single peak was observed at −1775 ppm, which agrees with the ClON2 environment around the platinum centre.

### 2.—Trans-[PtCl(NH₃)₂(Boc1,n)]⁺ (n=3, 4, 5)

Cis-[PtCl(ONO₂)(NH₃)₂] in DMF was treated with 0.8 equiv. of Boc1,n (n=3 (0.046 g, 0.266 mmol), 4 (0.050 g, 0.266 mmol), 5 (0.099 g, 0.266 mmol)) and 0.8 equiv. of triethylamine (Et₃N). The reaction mixture was reacted overnight at room temperature. The formation of cis-[PtCl(NH₃)₂(Boc1,n)]⁺ was checked by ¹⁹⁵Pt NMR, directly from the reaction solution. A single signal was observed at −2399 ppm, which was assigned to a ClN3 environment around the platinum. The same signal was observed, at the same chemical shift, for the three used linkers Boc1,3; Boc1,4 and Boc1,5.

### 3.—Trans-[Pt(ONO₂)(NH₃)₂(Boc1,n)]⁺ (n=3, 4, 5)

Cis-[Pt(Cl)(NH₃)₂(Boc1,n)]⁺ was treated with 0.9 equiv. of AgNO₃ (0.051 g, 0.3 mmol) in DMF. The reaction mixture was stirred overnight, in the dark. After this time, AgCl was filtered off and the yellow solution, containing cis-[Pt(ONO₂)(NH₃)₂(Boc1,n)]⁺, was checked by ¹⁹⁵Pt NMR. A single signal was observed at −2133 ppm. This signal agrees with the ON3 environment around the platinum. The same chemical shift value was observed for the three used linkers.

### 4.—Trans-[Pt(ptx)(NH₂)₂(Boc1,n)]²⁺ (n=3, 4, 5) where PTX is Pentoxyfilline

Cis-[Pt(ONO₂)(NH₃)₂(Boc1,n)]⁺ was treated with 0.8 equiv. of PTX (0.074 g, 0.266 mmol) at 75° C., overnight in the dark. After overnight the formation of the desired species were checked by ¹⁹⁵Pt NMR. A signal was observed at −2487, −2484 and −2492 ppm for complexes RBo1,3, RBoc1,4 and RBoc1,5, respectively. These values are in agreement with a 4N environment around the platinum centre.

### 5.—Deprotection of the Boc Group

The deprotection of the amine group in the final step takes places by the overnight, treatment with 0.1M HCl of each of the complexes RBoc1,3, RBoc1,4 and RBoc1,5 at 50° C., in the dark. ¹H NMR proved the successful deprotection by the disappearance of the signal assigned to the tert-butyl group at 1.34 ppm.

Purification of the trans-[Pt(ptx)(NH₂)₂(Boc1,n)]²⁺ (n=3, 4, 5) where PTX is Pentoxyfilline Systems by HPLC

Purification via HPLC for all three trans-ULS systems was performed as a mixture of different species was initially formed. For all the complexes the purification was successful, as checked by MS and in ¹H-NMR.

### Modification of the NH₂ Group of the Linker

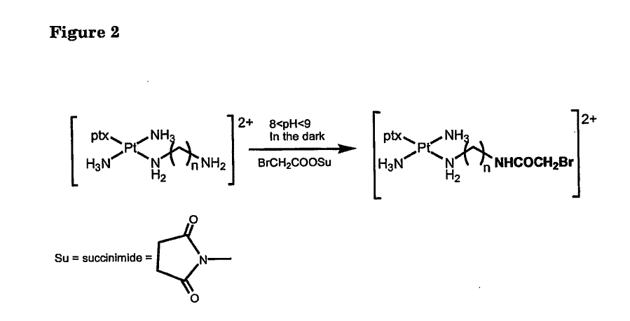

For the modification of the amine group, the 5. systems have been dissolved in water and the pH is adjusted between 8-9 with 1M NaOH. After the pH is adjusted, 1.5 equiv. of bromoacetic acid N-hydroxysuccinimide ester is added to the solution. The reaction is stirred at room temperature for 7 hr. It is important to maintain the pH of the reaction mixture between 8 and 9 during the complete reaction (FIG. 2), to allow the reaction between the amine group and the ester.

The progress of the reaction was followed by LCMS. Every 2 hr an aliquot was withdrawn and checked by LCMS. It was observed that the peak corresponding to the modified complexes was increasing its intensity with time. After 7 hr of reaction no further changes were observed in the chromatogram. The mass of the desired species were found to be 701.08, 715.05, 729.08, respectively. Purification of the modified complexes was required. It was performed by HPLC, with the same conditions as used for the purification of the non-modified complexes and was successful in all cases.

Subsequently, these complexes can be linked to a targeting device, such as but not limited to modified albumin, antibody, . . . .

Use of Trans-[PtCl(ONO₂)(NH₃)₂] as an Intermediate in the Synthesis of: AG1295-transULS:

AG1295 (18.7 μmol) was dissolved in DMF at a concentration of 10 mg/ml, and reacted with transULS 8.54 mol, 18.7 mM in DMF) at 50° C. for 1.5 h. The reaction was followed by HPLC. Mass Spectrometry analysis confirmed the presence of the target 1:1 drug:ULS species. The solvents may be removed under reduced pressure.

Mass spectrometry of K2-transULS (ESI+): calculated mass: 497 (m/z); detected masses: 497 [M⁺]

PTK787-transULS:

PTK787 (5.77 tμmol) was dissolved in DMF at a concentration of 10 mg/mL. An equimolar of trans-ULS (18.7 mM in DMF) was added and the resulting mixture was heated for 37° C. for 2 hours. An additional 0.25 molar equivalent of transULS then was added and the resulting solution was heated at 37° C. for 1.5 hour. Any excess of ULS may be removed by NaCl treatment as described in the synthesis of Y27632-cisULS. The solvents may be removed under reduced pressure. The reaction was followed by HPLC. Mass Spectrometry analysis confirmed the presence of the target 1:1 drug:ULS species.

Mass spectrometry of KTK787-transULS (ESI+): calculated mass: 609 (m/z); detected masses: 609 [M⁺].

### Losartan-transULS:

The product formed on treatment of losartan (3.40 μmmol; 10 mg/mL) with an equimolar of transULS in DMF (18.7 mM), overnight at ambient temperature. The reaction was followed by HPLC. Mass Spectrometry analysis confirmed the presence of the target 1:1 drug:ULS species.

Mass spectrometry of Losartan-transULS (ESI+): calculated mass: 710 (m/z); detected masses: 710 [M⁺].

SB202190-transULS:

The species was formed by reacting SB202190 (5.43 μmol) in DMF (10 mg/mL) with an equimolar of transULS in DMF (18.7 mM), for 1 hour at 37° C. The reaction was followed by HPLC. Mass Spectrometry analysis confirmed the presence of the target 1:1 drug:ULS species.

Mass spectrometry of SB202190-transULS (ESI+): calculated mass: 594 (m/z); detected masses: 594 [M⁺].

### 1.3. Synthesis and Characterisation of Dinuclear Species

### Dichloro(ethylenediamine)platinum (II) (1)

Potassium iodide (2 g, 12 mmol) was added to a solution of 1 g (2 mmol) of K₂PtCl₄ in 50 ml of water. The resulting dark solution of K₂PtI₄ was treated with 0.16 ml (2.4 mmol) of ethylenediamine and allowed to stand at room temperature for several hours. Then, the dark yellow precipitate of PtenI₂ was collected by filtration, washed with water, ice-cold ethanol and ether, and dried in air. Yield: 1 g (99%).

Silver nitrate (0.66 g, 3.9 mmol) in 5 ml of water was then added to the suspension of PtenI₂ (1 g, 2 mmol) in 25 ml of water upon stirring. The mixture was stirred in the dark overnight at room temperature. Then, the white precipitate of AgI was filtered off, and 0.33 g (4.4 mmol) of KCl was added to the filtrate. The mixture was stored overnight in the dark, and the resulting yellow precipitate was collected by filtration, washed with ice-cold water, ethanol and ether, dried in air. Yield: 0.52 g (80%). Anal. Calcd for C₂H₈N₂Cl₂Pt: C, 7.37; H, 2.47; N, 8.59. Found: C, 7.58; H, 2.80; N, 8.50. ¹⁹⁵Pt NMR (D₂O): δ −2330.

### 2-(2-(tert-butyloxycarbonylamino)ethoxy)ethanol (2a)

A solution of 2.62 g (12 mmol) of di-tert-butyl dicarbonate in 10 ml of ethanol was gently added to a stirred solution of 1.05 g (10 mmol) of 2-(2-aminoethoxy)ethanol in 10 ml of ethanol. After stirring for 4 h at room temperature, the solvent was removed under reduced pressure. The residue was extracted with 25 ml of ethylacetate, and the organic phase was dried (Na₂SO₄), filtered and evaporated to get the product. Yield: 1.95 g (95%). ¹H NMR (CDCl₃): δ 4.88 (s, 1H, NH), 3.74 (t, 2H, H₁), 3.56 (m, 4H, H₂+H₃), 3.34 (m, 2H, H₄), 1.45 (s, 9H, Boc).

### 1-(2-(tert-butyloxycarbonylamino)ethoxy)-2-sulfonylethane (2b)

Methanesulfonyl chloride (0.88 ml, 11.4 mmol) was gently added to a stirred solution of 1.95 g (9.5 mmol) of 2a and 2 ml (14.3 mmol) of triethylamine in 50 ml of dichloromethane at 0° C. The resulting solution was stirred for 1 h at 0° C. and for 30 min at room temperature. Then the solution was successively washed with 1M HCl (50 ml), water (50 ml), 10% aqueous solution of Na₂CO₃ (50 ml) and brine (50 ml). The organic phase was dried (Na₂SO₄), filtered and evaporated to get the product. Yield: 1.62 g (60%). ¹H NMR (D₂O): δ 4.35 (t, 2H, H₁), 3.73 (t, 2H, H₂), 3.56 (t, 2H, H₃), 3.33 (m, 2H, H₄).

### 1-(2-(tert-butyloxycarbonylamino)ethoxy)-2-azidoethane (2c)

Sodium azide (1.49 g, 22.9 mmol) was added to a stirred solution of 2b (1.62 g, 5.72 mmol) in 20 ml of anhydrous dimethylformamide. The mixture was heated at 60-70° C. overnight. After cooling, the solution was poured into water and extracted with ethyl acetate (5×30 ml). Organic phases were collected, dried (Na₂SO₄), filtered and evaporated to obtain the product. Yield: 0.6 g (46%). ¹H NMR (CDCl₃): δ 3.65 (t, 2H, H₂), 3.55 (t, 2H, H₃), 3.36 (m, 4H, H₁+H₄).

### 1-(2-(tert-butyloxycarbonylamino)ethoxy)-2-aminoethane (NONBoc, 2d)

A solution of 0.82 g (3.1 mmol) of triphenylphosphine in 10 ml of tetrahydrofurane was added to a solution of 0.6 g (2.6 mmol) of 2c in 20 ml of tetrahydrofurane. The resulting solution was stirred for 2 h at room temperature. Subsequently, 200 ml of water was added, and the mixture was stirred overnight at room temperature. Then the mixture was evaporated to 75 ml and filtered. The filtrate was washed with 25 ml of dichloromethane and evaporated to get the product. Yield: 0.5 g (79%). ¹H NMR (CDCl₃): δ 3.50 (m, 4H, H₂+H₃), 3.33 (m, 2H, H₄), 2.86 (t, 2H, H₁). ESI-MS: m/z 205.2 (M+H).

### [Pt(ethylenediamine)Cl(NO₃)] (1a)

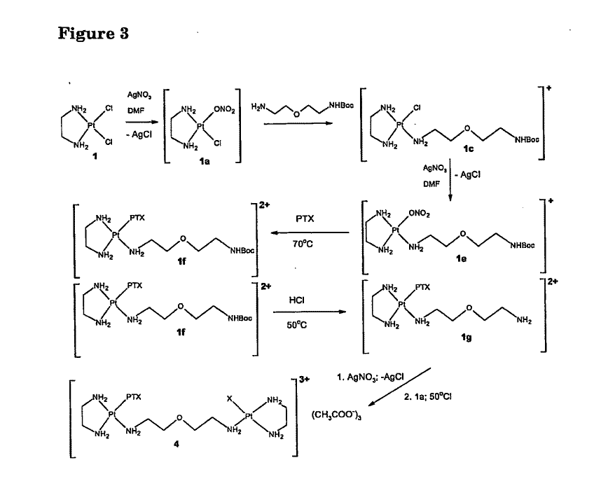

A solution of 38.6 mg (0.23 mmol) of AgNO₃ in 1 ml of dimethylformamide (DMF) was added portion wise over 1.5 h to a stirred solution of 78.2 mg (0.24 mmol) of 1 in 1.6 ml of DMF at room temperature in the dark (FIG. 3). The mixture was stirred for 5 h in the dark, and the AgCl precipitate was then filtered off. The resulting pale-yellow DMF solution of [PtenCl(NO₃)] was used as a starting material for the preparation of 1b and 1c as described below.

### [Pt(ethylenediamine)Cl(NONBoc)](NO₃) (1c)

A solution of 1a obtained as above was added to 40 mg (0.2 mmol) of NONBoc (2d) in the dark. The resulting solution was stirred overnight in the dark at room temperature. Then DMF was removed in vacuo. Excess of water was added to the residue, and a yellow precipitate of PtenCl₂ was filtered off. The remaining filtrate of 1c was lyophilized. Yield: 87 mg. ¹⁹⁵Pt NMR (H₂O): δ −2645.

### [Pt(ethylenediamine)(PTX)(NONBoc)](NO₃) (10, Route II where PTX is pentoxyfilline

A solution of 19.8 mg (0.117 mmol) of AgNO₃ in 1 ml of DMF was added portionwise over 1.5 h to a stirred solution of 68.6 mg (0.123 mmol) of 1c in 1.6 ml of DMF at room temperature in the dark. The mixture was stirred for 5 h in the dark, and the AgCl precipitate was then filtered off. The filtrate 1e was added to 27.4 mg (0.098 mmol) of PTX. The resulting solution was stirred in the dark at 70° C. Then it was filtered off the black precipitate, and evaporated. The residue was dissolved in water, filtered, and the filtrate of 1f was lyophilized. Yield (crude product): 71 mg. ¹⁹⁵Pt NMR (DMF): δ −2700. ESI-MS: m/z 737.2 (M-2(NO₃)-H), 459.28 (M-2(NO₃)-PTX-H).

### [{Pt(ethylenediamine)(PTX)}(μ-NON){Pt (ethylenediamine)X}](CH₃COO)₃, X═Cl (4) or CH₃COO (4a)

Complex 1f (81.4 mg, 0.094 mmol) was dissolved in 4 ml of 0.1M hydrochloric acid, and the resulting solution was heated overnight in the dark at 50° C. After that, pH of the solution was adjusted to 8 with 2M NaOH. Then 68 mg (0.4 mmol) of silver nitrate was added, in order to remove chloride ions from the solution. The precipitate of AgCl was filtered off, and subsequently a solution of 1a obtained as described above was added to the filtrate. The resulting solution was stirred at 50° C. for 24 h in the dark. The solvent was then removed in vacuo. The residue was dissolved in water, filtered and purified by high performance liquid chromatography. The purified product was lyophilized and characterized by ¹H and ¹⁹⁵Pt NMR and LC ESI-MS. Yield: 5 mg. ¹H NMR (D₂O): δ 8.61 (s, 1H, ptx), 4.50 (s, 3H, ptx), 4.09 (s, 3H, ptx), 4.01 (t, 2H, NON), 3.72 (t, 2H, NON), 3.64 (m, 4H, NON), 2.81 (m, 2H, en), 2.78 (m, 2H, en), 2.65 (m, 4H, ptx), 1.93 (s, CH₃COO), 1.62 (m, 4H, ptx). ¹⁹⁵Pt NMR (D₂O): δ −2400 (4a), −2632 (4), −2700. LC ESI-MS: retention time for 4 9.68 min (m/z 985.14 (M(4)³⁺-2H+CH₃COO), 647.04 (M(4)³⁺-3H-ptx); retention time for 4a 12.09 min (m/z 1009.08 (M(4a)³⁺-2H+CH₃COO), 669.93 (M(4a)³⁺-3H-ptx).

The above product is only an intermediate complex. Next, a targeting moiety will be linked to this product.

### 1.4. Synthesis and characterisation of heterobifunctional linkers.

### Synthesis of SMCC-ULS

20.2 mg of KRE-Boc-ULS (3.54*10⁻⁵ mol) were dissolved in 577 μL of a 200 mM solution of hydrochloric acid in MilliQ. The resulting solution was heated at 50° C. overnight. 264 of 200 mM hydrochloric acid in MilliQ then were added. The pH was adjusted to about 7.5 using a 1M solution of sodium hydroxide in MilliQ and a 200 mM solution of hydrochloric acid in MilliQ. The pH of a 0.05 M phosphate buffer was adjusted to 7.25 using a 1M solution of sodium hydroxide in MilliQ. 561 μL of the resulting buffer solution then were added to the solution of deprotected KRE-Boc-ULS. 11.9 mg (3.56*10⁻⁵ mol) of the SMCC succinimidyl ester were dissolved in about 5 drops of N,N′-dimethylformamide and added dropwise to the ULS solution. Precipitation appeared to occur. Therefore, DMF was added dropwise until solubility was achieved. The resulting solution was stirred overnight at ambient temperature under protection from light. Mass Spectrometry analysis then was performed.

### Synthesis of RGD-SMCC-ULS-TRAIL

The following example describes the conjugation of an RGD-peptide homing device to the recombinant therapeutic protein TRAIL (tumor necrosis factor [TNF]-related apoptosis-inducing ligand). TRAIL is a potential candidate for the eradication of cancer cells. Equipment of TRAIL with RGD-peptides can facilitate its homing to the tumor vasculature.

Human hisTRAIL was diluted to a concentration of 0.7 mg/ml in PBS containing 10% glycerol. SMCC-ULS was dissolved at a concentration of 10 mg/ml in 20 mM NaCl; all samples were adjusted to pH 7.4. His-TRAIL (500 ug, 7.6 nmol) was incubated with SMCC-ULS (76 nmol) for 4 hours at 37° C., after which unreacted SMCC-ULS was removed by dialysis against PBS 10% glycerol at 4° C. The RGD-peptide c(RGDf(ε-S-acetylthioacetyl)K) was dissolved at 10 mg/ml in a 1:4 acetonitrile/water mixture. The RGD-peptide (152 nmol) was added dropwise to the reaction mixture, after which hydroxylamine was added to a final concentration of 50 mM. The mixture was incubated overnight at room temperature while protected from light. Remaining SMCC groups were quenched by addition of cysteine (20:1 molar excess over the amount of TRAIL). The product RGD_SMCC-ULS-TRAIL was finally purified by dialysis against PBS/10% glycerol and stored at −20° C.

### Synthesis of Bio-ULS-TRAIL

The following example describes the synthesis of biotin-ULS-TRAIL, which can be complexed with RGD-equipped carriers via avidin-biotin interaction, as exemplified by RGD-PEG-avidin. The resulting bio-ULS-TRAIL//RGD-PEG-avidin complexes can interact with angiogenic endothelium and by this redirect TRAIL to tumor cells and tumor blood vessels.

### Synthesis of bio-ULS-TRAIL

Bio-ULS-TRAIL was prepared in a similar protocol as described above, using biotin-ULS at a 10:1 molar ratio over the amount of hisTRAIL. The final product was purified by dialysis against PBS/10% glycerol and stored at −20° C. Incorporation of biotin was conformed by SDS-PAGE and anti-biotin Western blotting using streptavidin-peroxidase.

### Synthesis of RGD-PEG-avidin

RGD-PEG-avidin was prepared in an analoguous protocol as described for RGD-PEG-HSA, using 2 mg avidin (30 μmol), a 10-fold molar excess of heterobifunctional vinylsulfone-polyethylene glycol-N-hydroxysuccinimide ester (1 mg, 300 nmol) and an 11-fold molar excess of RGD-peptide. An average of 5.4 RGD groups was incorporated per avidin.

Complexation of biotin-TRAIL with RGD-PEG-avidin

Complexes were made by premixing biotinylated TRAIL with RGD-PEG-avidin or were formed in situ, i.e. after adding both components consecutively to cells.

### Example 2

### In Vitro Evaluation of Cell Targeting Complexes

### 2.1. Drug Release Studies

The stability of the drug-carrier linkage was investigated by incubating cell targeting complexes at 37° C. under several conditions, e.g. buffers, buffers spiked with drug releasing substances or biological media. Typically, released drug was analyzed by HPLC and expressed as percentage of the total amount of conjugated drug/targeting complex.

### Results

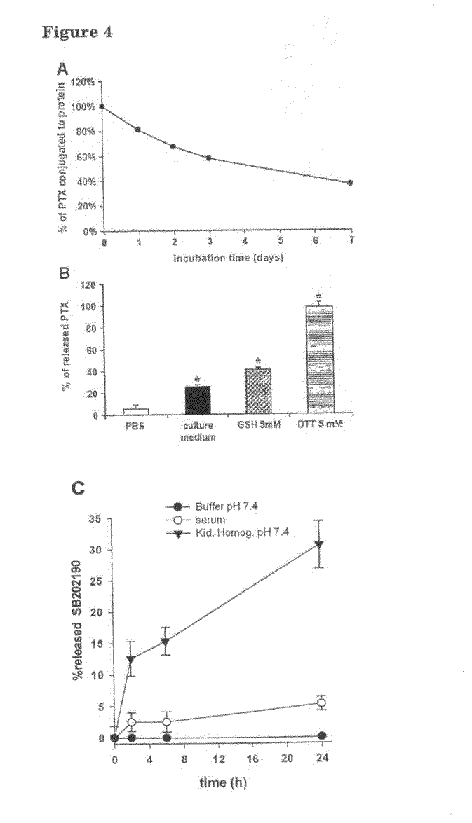

Typical examples of drug release profiles are depicted in FIG. 4.

Panel A: Drug release profile of PTX-cisULS-M6PHSA upon incubation at 37° C. in serum/PBS 1:1. PTX-cisULS-M6PHSA only slowly released drug in serum indicating that the linkage between drug and carrier displays adequate stability which will enable the conjugate to reach target cells. Alternatively, the observed release profile would allow for a slow release profile of the drug from a drug carrier that is not actively binding to specific target cells. Such a product could liberate the drug continuously, thereby controlling drug levels within the body. Examples of such preparation are for instance a subcutaneously injected depot preparation or a carrier that circulates in the blood stream.

Panel B: Release of PTX from PTX-cisULS-M6PHSA after 24 h incubation at 37° C. in buffer, HSC culture medium, or PBS with indicated compounds.

*: p<0.05 vs PBS.

As can be appreciated from this figure, drug was released in an environment that resembles intracellular conditions. Thus, the parent drug PTX can, in principle, be generated from the conjugate inside target cells.

Panel C: Release of SB202190 from SB202190-cisULS-LZM upon incubation at 37° C. in PBS pH 7.4, rat serum, or kidney homogenate in PBS 1/3 (w/v). SB202190-cisULS-LZM displayed high stability in buffer or serum, but continuously released free drug upon incubation in kidney homogenate. These results are in good agreement with the intended application of the product, i.e. the complex is capable of releasing its cargo (the coupled drug) after accumulation in target tissue.

### 2.2. In Vitro Effect Studies

The pharmacological effects of the conjugates were tested on different cultured cell types, e.g. activated hepatic stellate cells (HSC), kidney tubular cells (HK-2 cells) or human umbilical cord endothelial cells (HUVEC). Typically, cells were incubated for 24 h at 37° C. with the conjugates. Subsequently, the cells were subjected to analyses related to pharmacological activity of the coupled drug (gene expression analysis, immunohistochemcial evaluation of effect markers), or assays related to potential cytotoxicity of the complexes (cell viability assays, apoptosis assays).

### Results

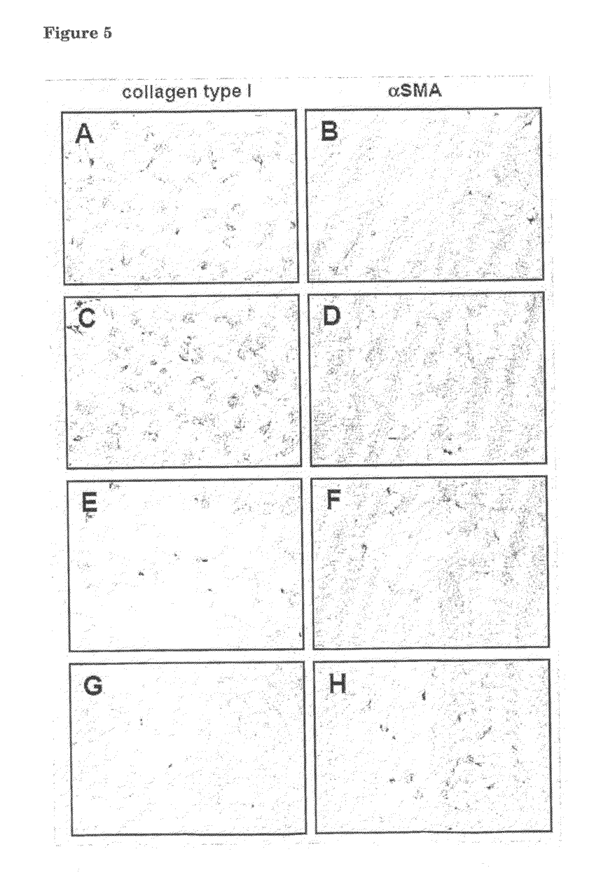

Typical examples of the pharmacological profiles of the cell targeting complexes are depicted in FIG. 5-14.

FIG. 5. Effects of PTX-cisULS-M6PHSA on fibrosis markers collagen I and α-smooth muscle actin. HSC were incubated with PTX-cisULS-HSA, PTX-cisULS-M6PHSA (both at 1 mg/ml, equivalent to 100 μM PTX,) or 1 mM non-targeted PTX. Cells were stained for collagen type I or α-smooth muscle actin and counterstained with hematoxillin. Panel A,B) controls; panel C,D) PTX-cisULS-HSA; panel E,F) PTX-cisULS-M6PHSA; panel G,H) non-targeted PTX. Magnification 10×. One of the hallmarks of liver fibrosis is the induced expression and production of a-smooth muscle actin (aSMA) and collagen type 1 by activated HSC. These proteins were therefore selected as read-out parameters of the antifibrotic effects of the targeted PTX. As can be seen in FIG. 5, activated HSC express collagen type I in a granular staining pattern in the cytoplasm, probably corresponding to the presence of procollagen type I, while aSMA stained in a fiber-like pattern (panel A,B). Treatment with PTX in a concentration of 1 mM reduced the intensity of both collagen type I production and aSMA (panel G,H). Incubation of the cells with 1 mg/ml PTX-cisULS-M6PHSA, which corresponds to 100 μM conjugated PTX, affected the collagen type I expression after 24 h of incubation considerably, as can be observed in the reduced intensity of the staining (panel E). Although the aSMA-staining intensity was not affected, pronounced changes in the morphology of the stellate cells were observed after the incubation with PTX-cisULSM6PHSA. Especially after staining of the abundantly present aSMA, the changes in the structure of the cells became clearly visible (panel F). HSC reacted to the treatment of PTX-cisULS-M6PHSA with a loss of cytoplasmic volume, rounded cell shape and detachment of the cells from the surface of the plates. These effects were not observed after incubation with equivalent concentrations of PTX-HSA (panel C,D).

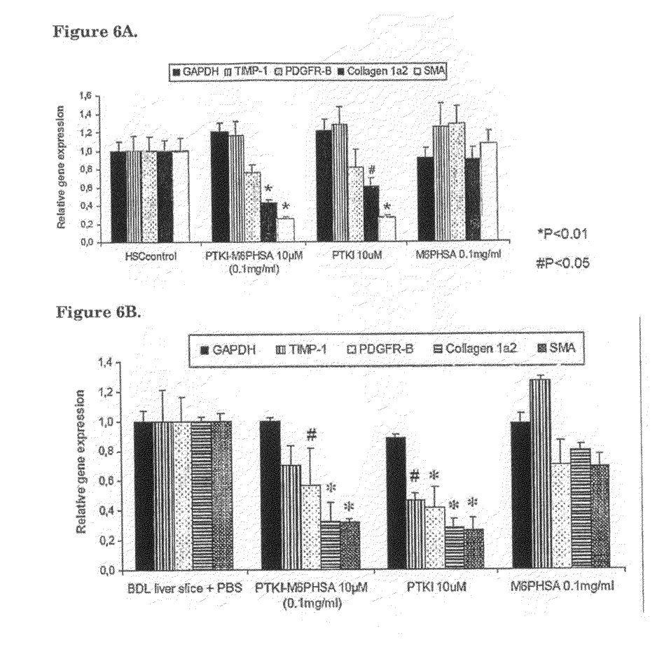

FIG. 6. Effects of PTKI-cisULS-M6PHSA on culture activated HSC (panel A) or liver slices from fibrotic rat livers (bileduct ligated rats, 3 weeks after ligation; panel B). Concentrations denote the amount of PTKI (10 μM) or the corresponding amount of M6PHSA carrier (0.1 mg/ml). Gene expression levels were normalized to the expression of GAPDH and subsequently normalized to the relative expression of control cells or slices.

The activity of PTKI and the cell targeting complex made thereof was studied by determination of gene-expression levels of fibrotic markers, which were analyzed by real-time quantitative RT-PCR. As can be appreciated from this example, PTKI-cisULS-M6PHSA exerted an antifibrotic effect similar to free drug PTKI, while carrier M6PHSA did not affect the investigated genes.

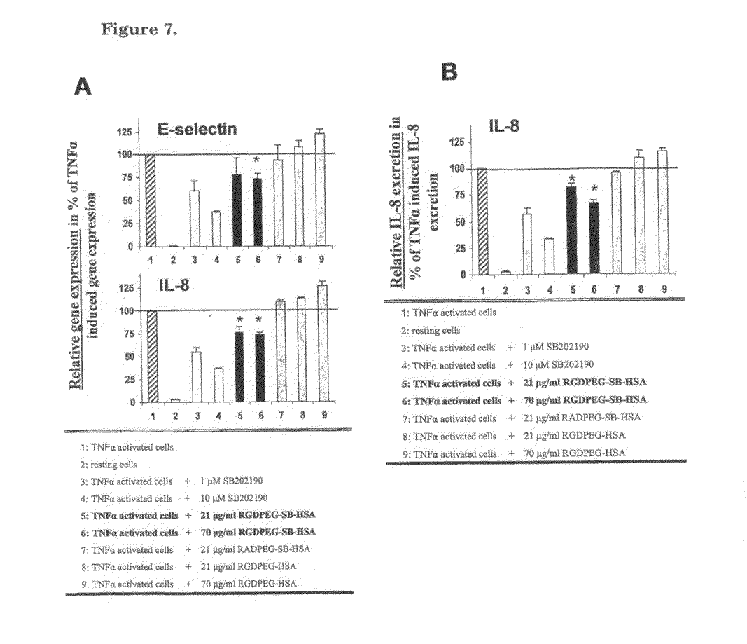

FIG. 7. Effect of RGD-PEG-SB202190-cisULS-HSA on inflammatory events in endothelial cells. HUVEC were preincubated with drug, drug targeting conjugate and control conjugates for 24 h after which TNFα was added. Cells and culture medium was harvested 24 h later and used for quantitative real time RT-PCR detection of inflammation-related genes or ELISA-based detection of secreted IL-8.

Panel A: Gene expression levels of IL8 in HUVEC treated with TNF and the described compounds. As can be appreciated, the IL8 and E-selectin genes are upregulated in HUVEC as response to treatment with TNF. This response can be inhibited with SB202190 at concentration of 10 μM to a level of 40%. Treatment with SB202190-cisULS-RGDPEG-HSA results in inhibition of this gene although to a lesser extent. Treatment with RGD-PEG-HSA carrier without drug did not reduce IL8 gene expression.

Furthermore, we demonstrated that inhibition of IL-8 gene expression also corresponded to a reduced production and secretion of IL8 in the culture medium (FIG. 7 panel B).

From these two results we conclude that the p38 MAPkinase inhibitor SB202190 can be generated from the cell targeting complex once the compound is processed by the cells. The observed difference in potency of the drug can be explained by the cellular routing of the conjugate, i.e. the drug targeting construct has to be internalized and processed in the lysosomal compartments of the cell to release the active drug. These processes take longer than the simple passive diffusion of the free compound. Combined with the different behaviour of the carrier in vivo however, the RGD-targeted drug may demonstrate a superior activity versus the free compound.

Light grey bars resemble free drug, black bars resemble drug targeting conjugate and dark grey bars resemble different control conjugates. 21 μg/ml of RGDPEG-SB-HSA equals 3 μM SB202190 and 70 μg/ml equals 10 μM SB202190. p<0.01 compared to respective control conjugates and to TNFα treated control.

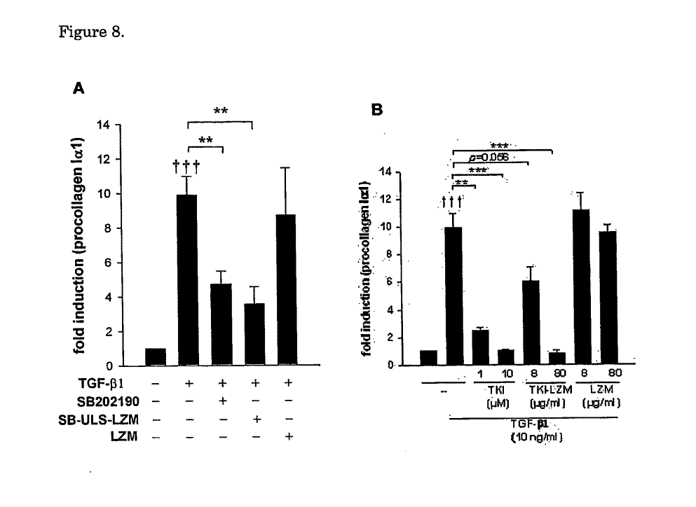

FIG. 8. Effects of SB-cisULS-LZM (panel A) and TKI-cisULS-LZM (panel B) on the gene expressions of procollagen-Ia1 induced by TGF-β1 in HK-2 renal tubular cells. Cells were grown to the 80% confluency and then deprived from serum for 24 h. SB-cisULS-LZM conjugate (155 μg/ml), TKI-cisULS-LZM conjugate (8 or 80 μg/ml) or methionine-modified LZM (equivalent concentrations) were incubated at the time of serum deprivation whereas free SB202190 (10 μM) or TKI were added 1 h before adding TGF-β1 (10 ng/ml) to the cells. The cells were further cultured for 24 h, after which RNA was isolated from the cells and mRNA expressions were determined by quantitative RT-PCR. Data represent the mean±SEM for at least 3 experiments. Differences versus control is presented as †††p<0.001. Other differences are **p<0.01 and ***p<0.001.

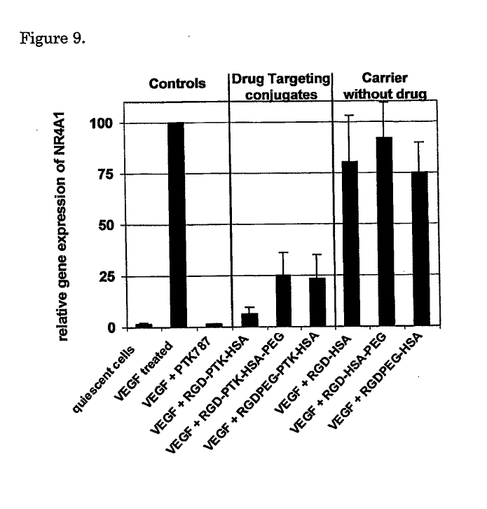

FIG. 9. Effects of RGD-equipped TK787-albumin conjugates on angiogenic responses in endothelial cells. Drug targeting conjugates were tested for their ability to inhibit VEGF induced gene expression. Cells were cultured in low-serum medium (1.5% FCS) and incubated with the indicated compounds. 5 ng VEGF was added to the wells after 24 h of incubation, and cell lysates were harvested after an additional 50 min. NR4A1, a nuclear receptor, was readily upregulated after addition of VEGF. All three drug targeting conjugates (approx. 4 ug/ml, equivalent to 500 nM of conjugated PTK787) were able to inhibit this upregulation significantly, while carrier without drug did not affect VEGF induced gene expression. Free drug was added at a concentration of 100 nM

### 2.3. Toxicity of Cell Targeting Complexes

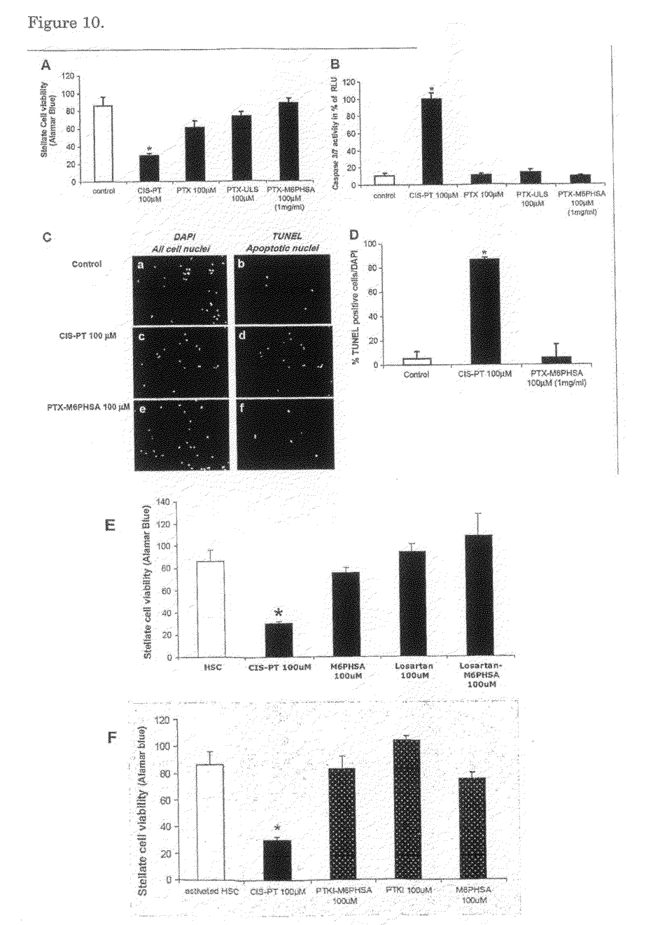

Since cisplatin is a well-known anti-tumor agent, the use of platinum-coordination chemistry for linking drugs to a carrier may infer unwanted toxicity into the construct. The effects of the conjugates on cell viability and apoptosis of target cells were therefore evaluated closely. FIG. 10, 11, 12 shows the results of cell viability studies and apoptosis assays of different types of target cells (HSC, renal tubular NRK-52E cells, and HUVEC, respectively) after their incubation with cell targeting complexes or free drug, free carrier/homing device or cisplatin (positive control). Incubations were carrier out for 24 h at 37° C. and cell viability was assayed by standard protocols using the Alamar Blue assay (HSC, HK-2 cells) or MTS assay (HUVEC). Apoptosis was assayed by either caspase 3/7 assay or TUNEL staining, according to standard protocols.

FIG. 10, Panel A-D: Incubation of HSC with PTX-cisULS-M6PHSA. A: cell viability of HSC; B: caspase 3/7 activity; C: TUNEL staining; D: quantification of TUNEL positive nuclei (TUNEL/DAPI ratio), * p<0.05 vs control. Panel E, F: Cell viability of culture-activated. HSC after incubation with losartan-cisULS-M6PHSA, and PTKI-cisULS-M6PHSA, respectively. Indicated concentrations reflect the platinum content of the conjugate, or equivalent amounts cisplatin, drug or M6PHSA. Treatment of activated HSC with cisplatin for 24 h induced apoptosis of this cell type, as reflected in all assays. In contrast, cell targeting complexes did not induce activation of apoptotic events nor a reduction in the number of viable HSC. From this, we conclude that the drug-cisULS-M6PHSA conjugates did not display cisplatin-like cytotoxic effects in HSC. * p<0.05 vs control.

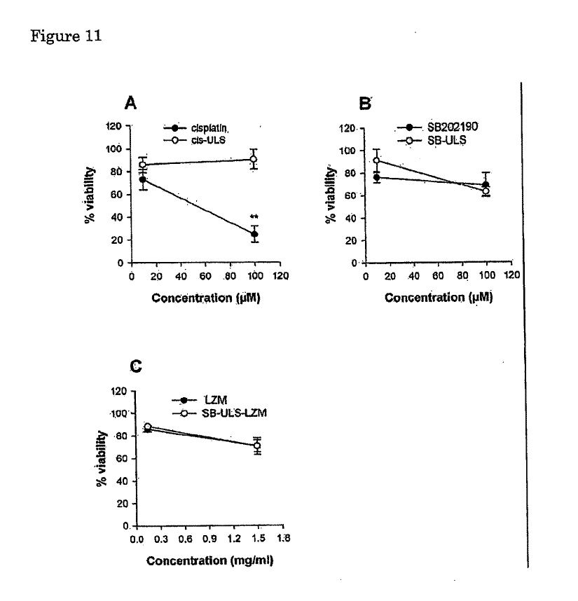

FIG. 11. Comparison of cellular toxicity of cell targeting complexes and cisplatin on renal tubular cells (NRK-52E). Since nephrotoxicity is one of the major side effects associated with cisplatin treatment, we carefully examined the safety of the cell targeting complexes directed to the kidney. For this purpose, we incubated drug-cisULS products and drug-cisULS-LZM conjugates with kidney proximal tubular cells and assayed the cell-viability after 24 h. Cells were incubated for 24 h with indicated compounds, after which cell viability was determined by the Alamar blue assay. Concentrations tested corresponded to 10 and 100 μM platinum, or were equivalent to the amount of drug (SB202190) or protein (LZM). **p<0.01. Treatment with drug-cisULS affected the cell viability similar as treatment with the parent drug. Similarly, SB202190-cisULS-LZM did not affect cell viability other than non-modified lysozyme. In contrast, cisplatin affected cell viability significantly, as expected from literature. From these results, we conclude that cell targeting complexes prepared with cisULS are not displaying platinum-related toxicity when incubated with renal tubular cells.

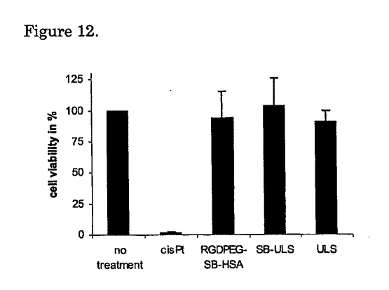

FIG. 12. RGDPEG-SB-HSA and ULS containing conjugates displayed no toxicity for endothelial cells. RGDPEG-SB-HSA (100 μg/ml), SB-ULS, ULS or cisplatin (all at 100 μM) were added to EC medium and incubated for three days. Cell viability was assessed in comparison to non treated control cells (=100% viability) using MTS assay.

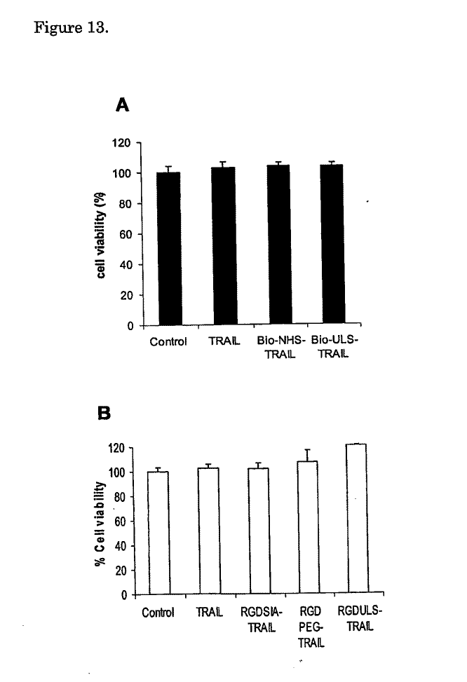

FIG. 13 shows that HUVEC cell viability is not affected by either non-modified hisTRAIL and biotinylated TRAIL (panel A) or RGD-equipped TRAIL (panel B). Incubations were performed for 48 h at a concentration of 100 ng/ml of the recombinant protein. In another example (see below) we will demonstrate that tumour cells are responsive to TRAIL and its derivatives. We therefore concluded that HUVEC are not sensitive to TRAIL, and that the absence of cytotoxicity in transition-metal based cell targeting complexes indicated the safety of the linker system. TRAIL: TNF-Related apoptosis Inducing Ligand; modified TRAIL was prepared using the commercially available bio-EZ reagent (BIO-NHS-TRAIL), using KRE-Bio-ULS (BIO-ULS-TRAIL); using iodoacetic acid NHS reagent (RGD SIA-TRAIL); using VNS-PEG-NHS 3.4 kDa linker (RGD PEG-TRAIL); using SMCC-ULS (RGDULS-TRAIL).

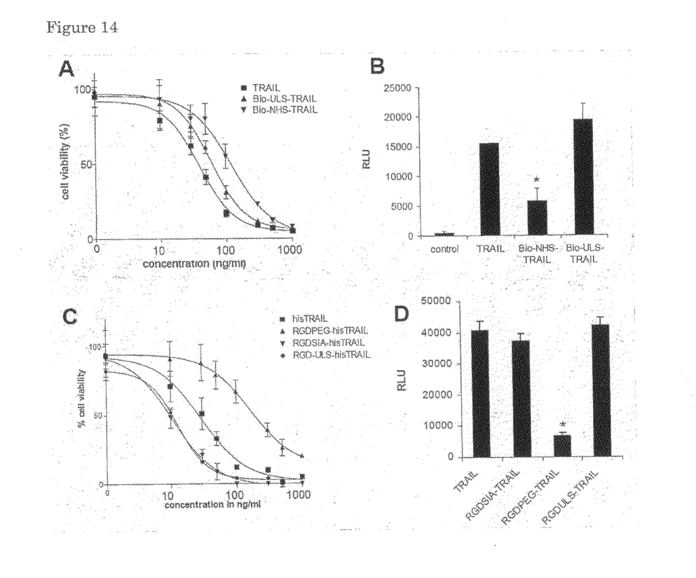

FIG. 14. The pharmacological activity of TRAIL and its cell targeting complexes was evaluated in Jurkat leukemic T-cells, by analyzing cell viability (48 h incubation) or induction of caspases assay (4 h incubation). Both types of experiments demonstrated that TRAIL and derivatives made of TRAIL were capable of killing tumour cells. Furthermore, derivatives prepared with ULS-based linkers displayed superior cytotoxic activity as compared to other chemical approaches for preparation of cell targeting complexes (panel A,B: superior activity of Bio-ULS-TRAIL over BIO-NHS-TRAIL; panel C,D: superior activity of RGD-ULS-TRAIL over RGD-PEG-TRAIL). We concluded that the therapeutic activity of TRAIL was conserved after modification of the protein with homing ligands. Panel A, B: Incubation of Jurkat cells with biotinylated TRAILs. A) Cell viability assay; B) Caspase activity assay. Panel C,D: Incubation of Jurkat cells with RGD-equipped TRAILs. C) Cell viability assay; D) caspase activity assay. *: p<0.05.

### 2.4. In Vitro Targeting Studies

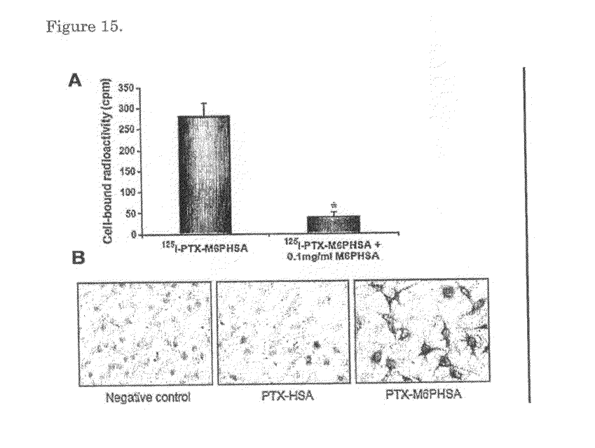

The interaction of cell targeting complexes with target cells was investigated in cultured target cells. Typically, in vitro targeting studies were performed by incubating target cells with the complexes, at either 4° C. or 37° C. Binding and/or internalization of the complexes by target cells was detected by either immunohistochemical staining for the complex (e.g. anti-HSA or anti-LZM staining) or by quantification of a radioactive reporter group that had been introduced in the complex (e.g. ¹²⁵I). Representative examples of targeting studies are depicted in FIGS. 15-17.

We studied the binding of ¹²⁵I-radiolabeled PTX-cisULS-M6PHSA to M6P/IGFII receptor expressing NIH/3T3 fibroblasts (incubation: 4 h at 37° C.). FIG. 15A shows that a tracer dose of the cell targeting complex bound to the cells and that binding could be displaced by excess of unlabeled M6PHSA. In addition, we demonstrated binding of PTX-cisULS-M6PHSA (1 mg/ml, 4 h at 37°) by anti-HSA immunodetection, while PTX-HSA did not bind to the cells (FIG. 15B). Thus, binding of PTX-M6PHSA to target cells was mediated via the M6P homing ligands in the conjugates.

Binding of RGD-equipped complexes to endothelial cells was studied using ⁸⁹Zr-radiolabeled compounds.

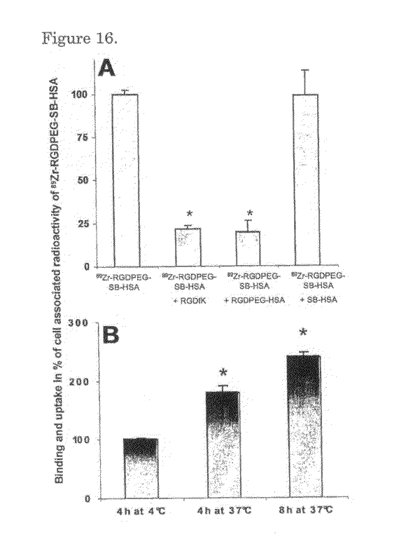

FIG. 16A shows that binding of RGDPEG-SB202190-cisULS-HSA to endothelial cells (HUVEC) could be displaced by free RGD-peptide, as well as by RGDPEG-HSA not equipped with SB202190. In contrast, SB202190-cisULS-HSA did not affect the interaction between complex and target cells. FIG. 16B shows that accumulation of the complex increased in time when cells and compounds were incubated at 37° C., which indicated receptor mediated internalization of the complex.

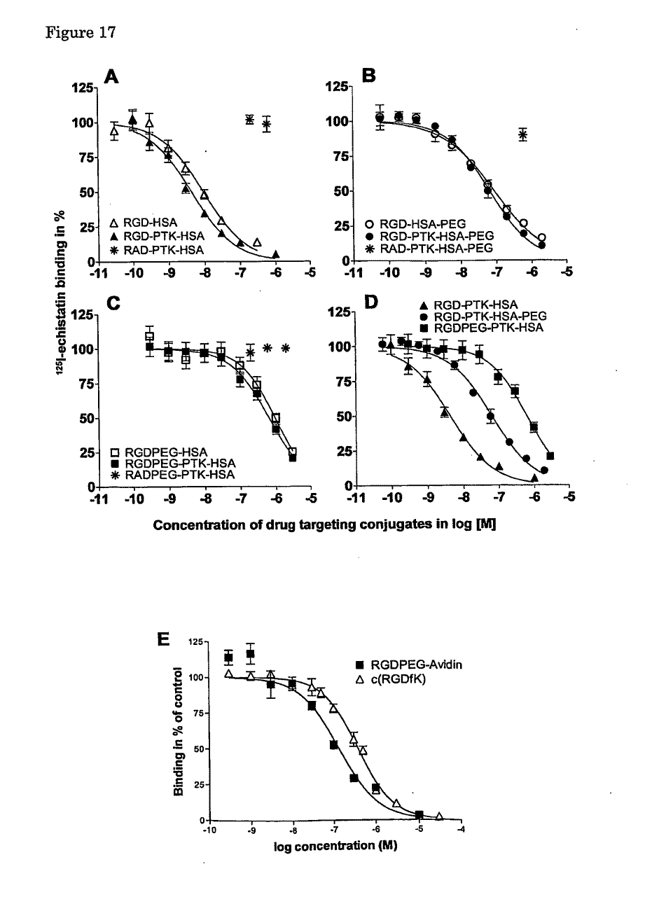

In a different experimental setup that allowed the calculation for binding constants, we explored the affinity of RGD-equipped PTK787-cisULS-albumins. ¹²⁵I-labeled echistatin was used as a radioligand for αᵥβ₃-integrin expressed on the surface of HUVEC. Competitive binding studies were performed by coincubating using confluent monolayers of HUVEC with the radiolabeled ligand and non-radiolabeled complexes. All RGD equipped conjugates completely displaced ¹²⁵I-echistatin, while conjugates modified with the control RAD peptide demonstrated no displacement (FIG. 17A-C). Furthermore, conjugation of PTK787-cisULS to the carrier did not hamper the interaction between homing ligand and target cells, since carrier with and without drug showed similar affinity. Highest binding affinity was determined for RGD-PTK-HSA (IC50: 4.4 nM; 0.3 μg/ml) followed by RGD-PTK-HSA-PEG (IC50: 65 nM, 4.4 μg/ml) and RGDPEG-PTK-HSA (IC50: 640 nM, 43 μg/ml) (FIG. 17D).

Interaction of RGDPEG-avidin with HUVEC was tested in a similar setup (IC50: 134 nM; FIG. 17E).

### Example 3

### In Vivo Evaluation of Cell Targeting Complexes

### 3.1. In Vivo Targeting Studies

In vivo targeting studies encompassed studies in which drug levels in the target organ or other tissues or body fluids (serum, urine) were quantified by HPLC. Typically, drug levels were determined by HPLC after release of the drug from the targeting complex, or free drug levels reflecting drug released from the complexes were assayed. Furthermore, accumulation of cell targeting complexes in tissues or cell types was determined by immunohistochemical staining for the complex (anti-HSA or anti-LZM staining) or by detection of radiolabeled complexes (¹²⁵I-radiolabeled conjugates).

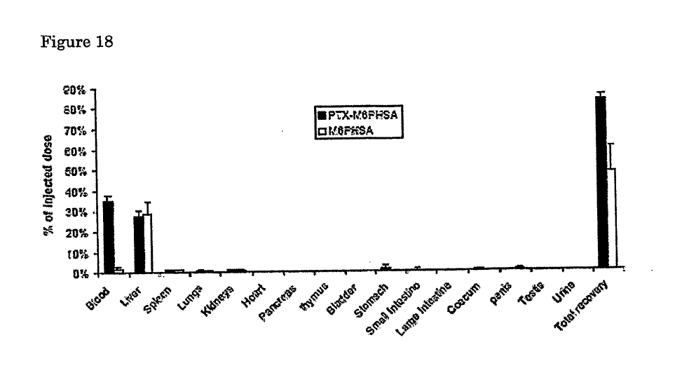

Typical examples are shown in FIGS. 18-22.

### 3.1.1. Pharmacokinetic Evaluation of Liver Directed Cell Targeting Complexes.

Organ distribution studies with this type of cell targeting complexes were performed in two well known animal models of liver fibrosis, the bile duct ligation (BDL) model [1] and the CCl₄ inhalation model [6], respectively. All in vivo experiments were approved by the Local Committee for Care and Use of Laboratory Animals.

### Evaluation of PTX-cisULS-M6PHSA

Pharmacokinetic studies were performed in animals with progressed fibrosis (3 weeks after BDL). At this time point, the liver showed severe fibrotic lesions and excessive matrix deposition, as assessed by immunostaining for collagen III and αSMA. During biodistribution studies, rats were kept under isoflurane anesthesia and body temperature was maintained at 37-38° C.

Rats were i.v. injected via the dorsal penis vein with tracer doses (106 cpm/rat) of either ¹²⁵I-PTX-cisULS-M6PHSA or ¹²⁵I-radiolabeled M6PHSA. At 10 minutes after administration, blood samples were taken by heart puncture and the organs were excised, washed in saline, and weighed after which radioactivity was counted. Urine was recovered from the bladder and measured. The total radioactivity per organ was calculated and corrected for blood-derived radioactivity using BDL correction factors. We observed a rapid distribution of ¹²⁵I-radiolabeled PTX-cisULS-M6PHSA and ¹²⁵I-radiolabeled M6PHSA to the fibrotic liver (FIG. 18). No accumulation in other organs was observed. In addition, non-radiolabeled PTX-cisULS-M6PHSA (2 mg/kg) was administered to BDL3 rats. Tissue specimens were processed for immtmohistochemical analysis according to standard procedures. Cryostat sections (4 μm) of the liver were fixed in acetone and stained for the presence of conjugates by anti-HSA immunodetection. We observed colocalization of PTX-cisULSM6PHSA with HSC (stained by anti-desmin), from which we concluded that PTX-cisULS-M6PHSA bound specifically to HSC.

Evaluation of losartan-cisULS-M6PHSA

Pharmacokinetic studies were performed in the BDL model and in the CCl₄ inhalation model. The fibrotic process in the CCl₄ inhalation model has completely different characteristics from the BDL model, both in underlying pathophysiology and speed of progression. Experiments were performed at intermediate disease-stages, i.e. 2 weeks after BDL and 9 weeks after start of CCL4 inhalation.

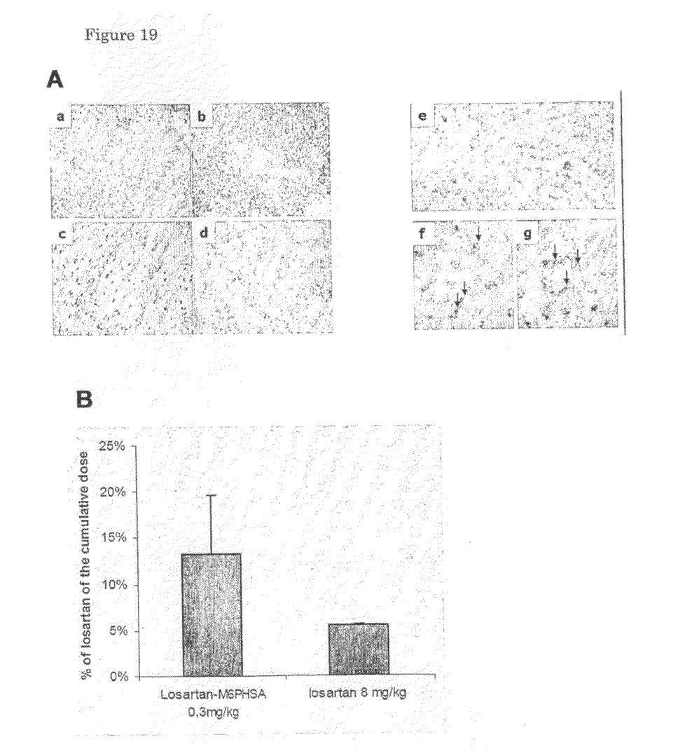

At days 12-15 after bile duct ligation, rats received intravenous injections of saline, losartan-cisULS-M6PHSA (3.3 mg/kg/day, corresponding to 125 μg losartan/kg), M6PHSA alone (3.3 mg/kg/day), or orally administrated losartan (5 mg/kg/day by gavage). Ten minutes after the last dose, animals were sacrificed and blood and liver samples were obtained. The presence of losartan-cisULS-M6PHSA or M6PHSA in different organs was determined by immunostaining using an anti-HSA antibody (FIG. 19A). Losartan-cisULS-M6PHSA was not detected in the lung (a), spleen (b), heart (c) or kidney (d) (magnification 4×) in rats treated with losartan-cisULS-M6PHSA, but was prominently detected in the liver within non-parenchymal cells of (e) (magnification 4×). Losartan-cisULS-M6PHSA co-localized with stellate cells in rat livers (arrows), as assessed with double immunostaining with anti-HSA and anti-desmin (f) (magnification 40×). The amount of losartan in liver tissue homogenates (0.3 g liver/ml of PBS, Turrax homogenization) was analyzed by HPLC, after liquid-liquid extraction of losartan using methyl butyl ether. Liver homogenates from animals treated with losartan-cisULS-M6PHSA were incubated overnight with 0.5M KSCN 80° C., in order to release losartan from the conjugate, while homogenates from free losartan treated animals were not treated with KSCN. The extraction was performed by adding 3 ml of methyl butyl ether to 200 μl of liver homogenate and vortexing for 5 min. Layers were separated by centrifugation at 900×g for 5 min and the aqueous layer was frozen in liquid nitrogen. The upper organic layer was transferred to another borosilicate glass tube and evaporated completely at 60° C. The extraction procedure was repeated twice and the total residue was reconstituted in 200 μl of mobile phase. Chromatography was carried out using a C18 (C18, 5 μm, 4.6×150 mm) reversed-phase column (Surefire, Waters Inc., Milford, Mass., USA) at 40° C. with an isocratic mobile phase consisting of acetonitrile-water-trifluoroacetic acid (30:70:0.1, v/v/v; pH 2.0).

While oral administration of free oral losartan provided an average tissue concentration of 12.1 μg/g liver, corresponding to 4% of the cumulative dose animals that were given losartan-cisULS-M6PHSA, exhibited losartan levels of 1.5 μg/g, corresponding to 20% of the cumulative dose (81% of the last injected dose). As can be appreciated from these results, losartan-cisULS-M6PHSA demonstrated a preferential liver distribution. Furthermore, drug levels in HSC are most likely much higher due to the cell-specific accumulation of the cell targeting complex and the fact that stellate cells constitute only a small fraction of the whole liver. Oral administered losartan does not show this preferential homing to HSC.

In the 9^{th} week after starting with the CCl₄ inhalation, rats were treated with four consecutive daily intravenous injections of saline, losartan-cisULS-M6PHSA (8 mg/kg, corresponding to 0.3 mg losartan/kg), M6PHSA alone (8 mg/kg), or free losartan (0.3 mg losartan/kg). Ten minutes after the last injection, animals were sacrificed and blood and liver samples were obtained and processed as described above. We observed a similar preferential distribution of losartan-cisULS-M6PHSA to the fibrotic liver whereas the anti-HSA staining in other organs was negative. Furthermore, losartan tissue levels demonstrated preferential distribution of losartan-cisULS-M6PHSA to the liver, in contrast to free losartan (FIG. 19B).

### Evaluation of PTKI-cisULS-M6PHSA

At day 10 after BDL, rats received a single intravenous injection of PTKI-cisULS-M6PHSA (3.3 mg/kg, corresponding to 150 μg PTKI/kg). Control animals were injected with an equivalent volume of the vehicle (saline) Animals were sacrificed 2 h post injection of the compounds, and organs were harvested and processed for immunohistochemical detection of the conjugate as described above for other drug-cisULS-M6PHSA conjugates. We observed accumulation of PTKI-cisULS-M6PHSA in the liver, while the construct was undetectable in other organs like heart, kidney, lung and spleen.

### 3.1.2. Pharmacokinetic Evaluation of Kidney Directed Cell Targeting Complexes.

Organ distribution studies with this type of cell targeting complexes were performed in healthy Wistar rats. All in vivo experiments were approved by the Local Committee for Care and Use of Laboratory Animals.

Evaluation of SB202190-cisULS-LZM

Rats were injected intravenously with a single dose of the SB202190-cisULS-lysozyme conjugate (16 mg/kg equivalent to 376 μg/kg of SB202190; dissolved in 5% glucose) via the penile vein. Animals were sacrificed at different time points between 1 and 72 h after administration. Blood samples were collected by heart puncture and kidneys were isolated after flushing the organs gently with saline. Serum and organs were snap-frozen into liquid nitrogen immediately. Urine samples were collected using metabolic cages and combined with the urine collected from urinary bladder after sacrificing the animals. Kidneys were weighed, homogenized (1:3 w/v in PBS, Turrax homogenization) and then stored at −80° C. Released drug amounts were estimated by HPLC analysis after extraction as described before [7]. To estimate total drug (bound plus released), samples were treated with KSCN to release SB202190 from the ULS linker as described above and then subjected to HPLC analysis. Anti-LZM immunohistochemical staining was performed on cryostat kidney sections to detect the uptake of the conjugate in tubular cells.

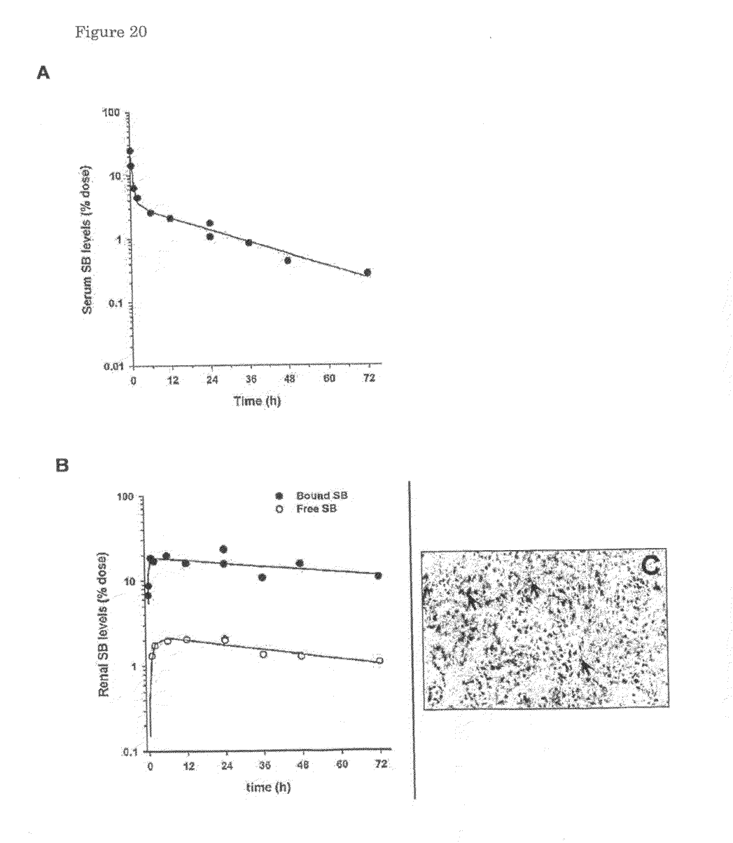

In prior studies with other drug-LZM conjugates, we observed a rapid renal accumulation of these products after intravenous administration [4]. We used this knowledge to design an optimized protocol for the pharmacokinetic studies with drug-cisULS-LZM conjugates that allowed optimal estimation of pharmacokinetic parameters. The serum-disappearance curve of SB202190-cisULS-LZM is shown in FIG. 20A. Only carrier-bound SB202190 was detected in the serum, while free drug was absent at all time points. From this result, we concluded that the conjugate remained stable in the serum.

Furthermore, SB202190-cisULS-LZM accumulated efficiently in the kidneys within 1 h following the intravenous injection, amounting to a total of 20% of the injected dose (FIG. 20B). Remarkably, we observed continuous levels of both free drug and bound drug during a three-day period after a single dose. This profile can be explained by the renal accumulation of the product, which subsequently forms a depot that generates free drug by slow drug release from the linker. In contrast, after administration of 5 mg/kg of free SB202190, only 0.2% distributed to the kidney at 4 h after administration [4]. This difference between free drug and SB202190-cisULS-LZM clearly illustrates the potential of the developed cell targeting complex in providing selective enrichment of drugs in specific tissues.

The accumulation of the conjugate in proximal tubular cells was confirmed by anti-LZM immunohistochemical staining on kidney sections (FIG. 20C). Arrows denote the accumulation of SB202190-cisULS-LZM in tubular cells.

Evaluation of TKI-cisULS-LZM and Y27632-cisULS-LZM

In a similar protocol as described above, we studied the organ distribution of TKI-cisULS-LZM (20 mg/kg dissolved in 5% glucose) and Y27632-cisULS-LZM (20 mg/kg, dissolved in 5% glucose). Renal drug levels were assayed in KSCN-treated kidney homogenates and serum samples using HPLC methods optimized for the respective drugs. Y27632 was analyzed on a C18 reversed-phase SunFire™ column C18 using a mobile phase of water-methanol-trifluoroacetic acid (86:14:0.1, v/v/v; pH 2.0) at a flow rate of 1 ml/min. TKI was analyzed on the same column using a mobile phase consisted of water-acetonitrile-trifluoroacetic acid (91:09:0.1, v/v/v; pH 2.0) at a flow rate of 1 ml/min.

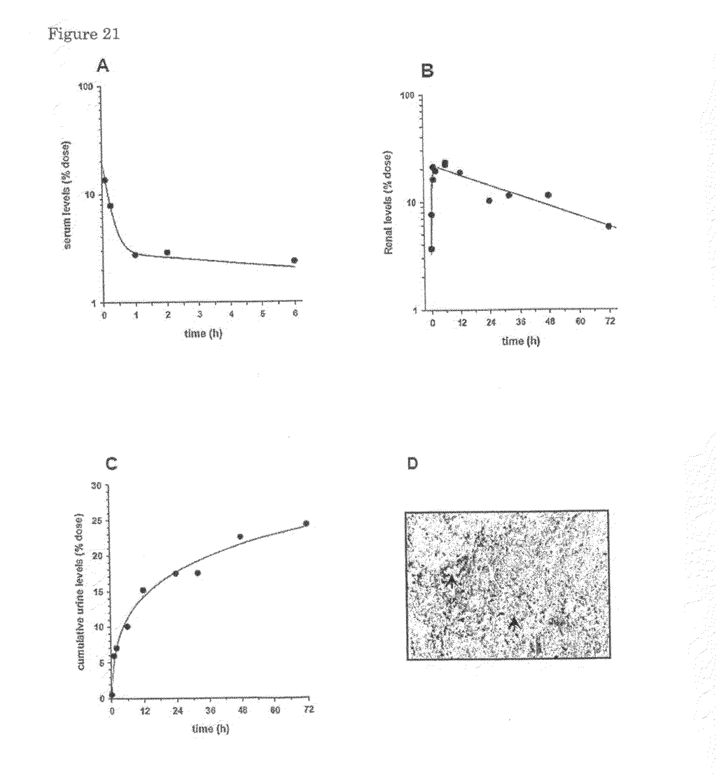

FIG. 21 shows the results obtained with TKI-cisULS-LZM. Panel A: serum disappearance; panel B: renal drug levels; panel C: cumulative excretion in urine. Symbols represent the % dose of TKI at each time point; the continuous line represents the pharmacokinetic data-fit curve (two-compartment model). Panel (D) represents the localization of the conjugate in tubular cells at 1 h by anti-LZM immunohistochemical analysis (magn. 200×).

As can be appreciated from these data, TKI-cisULS-LZM distributed rapidly and extensively to the kidneys and provided renal drug levels by local drug release in an analogous manner as observed for SB202190-cisULS-LZM. Metabolites of the conjugate were excreted in the urine as can be observed in panel C.

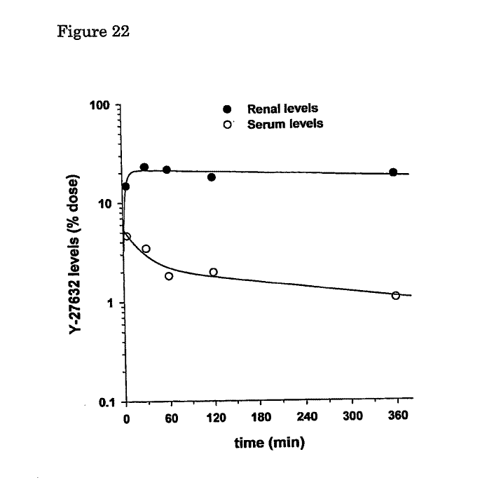

Pharmacokinetic studies with Y27632-cisULS-LZM provided similar results, yielding renal levels of approximately 20% of the injected dose at 1 h after administration and onwards (FIG. 22).

### 3.2. In Vivo Effect Studies

In vivo effect studies encompassed studies in which the pharmacological activity and potential therapeutic activity of cell targeting complexes was investigated by studying disease-related parameters. With respect to cell targeting complexes directed at HSC in the fibrotic liver, we investigated the deposition of extracellular matrix components (Sirius Red staining) the HSC marker αSMA, the influx of immune cells (CD43 staining). With respect to kidney directed cell targeting complexes for the treatment of renal fibrosis, we evaluated renal morphology, the expression αSMA, influx of macrophages (ED-1 staining) and activation of p38-MAPkinase (phospho-p38 staining). In both approaches, gene-expression levels were quantitatified by real-time RT-PCR using pre-designed Assays-on-Demand TaqMan probes.

In addition to drug related effects, we investigated whether administration of cell targeting complexes to healthy rats induced potential platinum related toxicity. Since the kidney is one of the major sites of platinum toxicity of cytostatic compounds, we performed these studies with SB202190-cisULS-LZM which accumulates in the kidney. We investigated morphology and induction of apoptosis (TUNEL staining), as well as basic renal parameters.

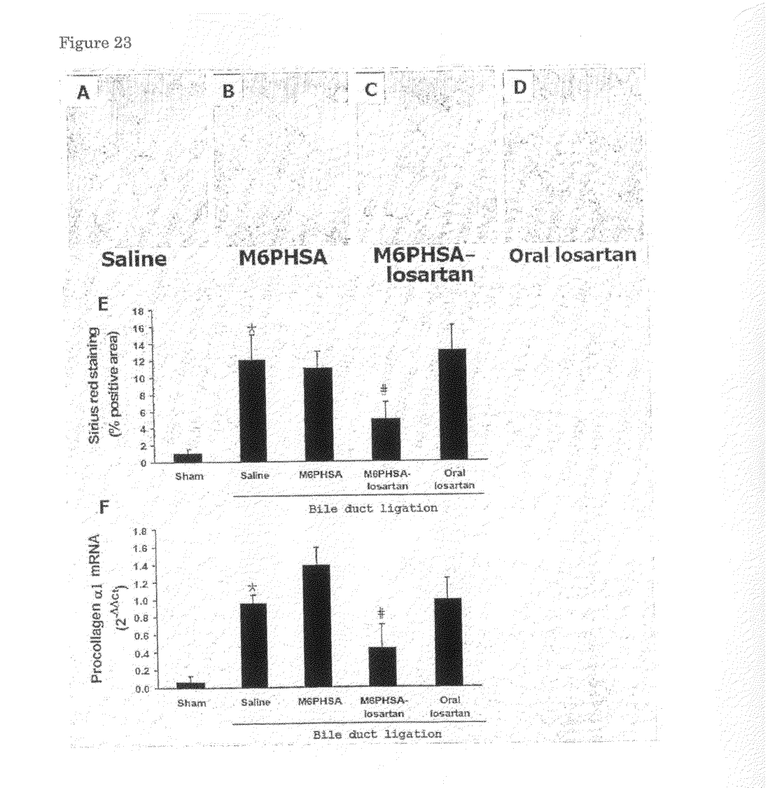

Typical examples of in vivo effect studies are shown in FIGS. 23-30.

### 3.2.1. Evaluation of Liver-Directed Cell Targeting Complexes.

Antifibrotic effects of losartan-cisULS-M6PHSA and PTKI-cisULS-M6PHSA were studied in the BDL model of liver fibrosis. In addition, losartan-cisULS-M6PHSA was evaluated in the CCl₄ inhalation model of liver fibrosis. Animal experiments were performed as described above. Losartan-cisULS-M6PHSA treated animals received four daily doses and were sacrificed 10 min after the last dose. PTKI-cisULS-M6PHSA treated animals received a single dose, and were sacrificed 24 h or 48 h after receiving the compound. Cryostat sections or paraffin-embedded sections were processed for immunohistochemical staining according to standard procedures, and positively stained areas were quantified by morphometric analysis. The degree of hepatic fibrosis was estimated as the percentage of area of each section stained positive with picro Sirius Red. The amount of fibrogenic myofibroblasts was estimated by measuring the percentage of area positively stained for αSMA. The amount of infiltrated inflammatory cells was estimated by quantification of anti-CD43 immunostaining. The cumulated data together demonstrate that this type of cell targeting complexes can intervene in liver fibrosis, and even reverse fibrotic processes. Furthermore, the effects can be obtained at considerable lower doses of drug as reported for the non-delivered drugs.

FIG. 23. Effect of losartan-cisULS-M6PHSA at hepatic fibrosis in BDL rats (Sirius Red staining in paraffin embedded sections and quantification). Severe bridging was observed in rats receiving saline (A), M6PHSA (B) and oral losartan (D). However, rats treated with losartan-cisULS-M6PHSA (C) showed fewer areas with collagen accumulation (magnification 4×). (E): Quantification of the area with Sirius Red staining in liver specimens (n=10, mean±SEM, statistical analysis: *P<0.05 vs sham; #P<0.05 vs saline, MP6PHSA and oral losartan). (F): Quantification of the expression of procollagen α1(II) gene-expression in rat livers Mann-Whitney test between saline and drug treated groups: *P<0.05 vs sham; #P<0.05 vs saline, MP6PHSA and oral losartan.

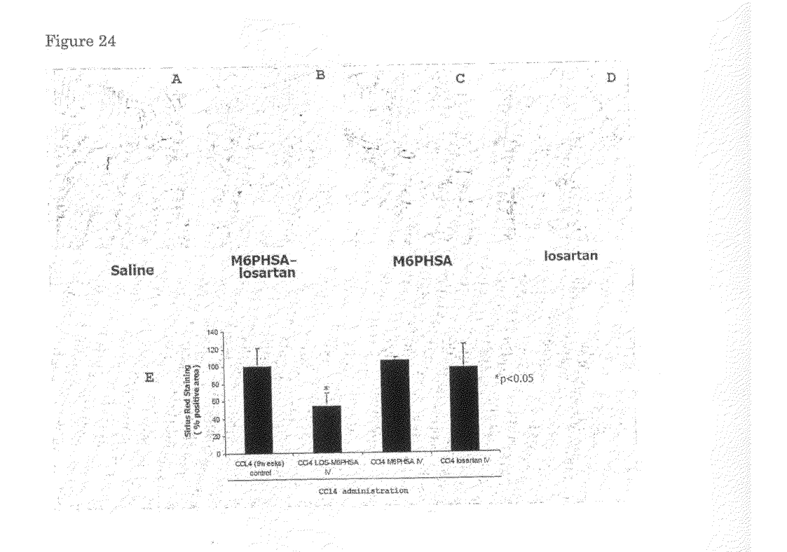

FIG. 24. Effect of losartan-cisULS-M6PHSA at hepatic fibrosis in CCl₄ rats (Sirius Red staining in paraffin embedded sections and quantification). CCL4 rats were treated with: A. Saline, B. M6PHSA, C. losartan-cisULS-M6PHSA and D. free losartan. (E): Quantification of Sirius Red positive area. *P<0.05 vs control fibrotic rats.

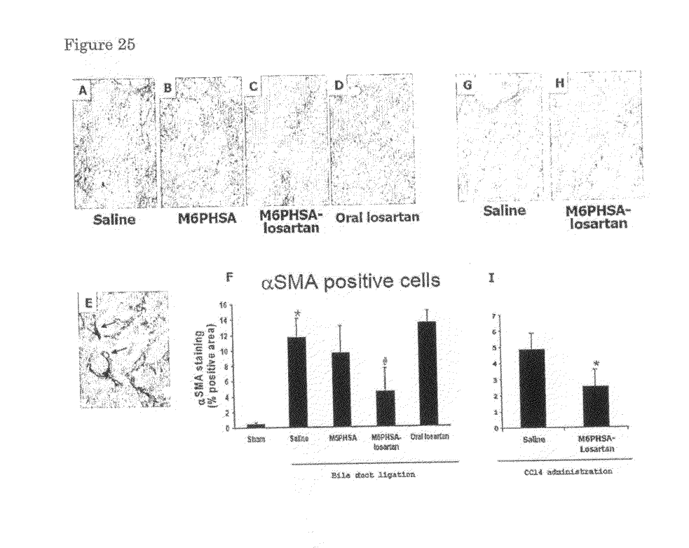

FIG. 25. Effect of different treatments on the accumulation of myofibroblasts and activated hepatic stellate cells, as assessed by expression of αSMA. BDL rats receiving saline (A), M6PHSA (B) or oral losartan (I)) showed a marked accumulation of αSMA-positive cells, which co-localized with areas with active fibrogenesis. However, rats treated with losartan-cisULS-M6PHSA (C) showed a marked reduction in αSMA-positive cells (magnification 4×).

(E). High magnification (400×) photomicrograph of a liver from a bile duct ligated rat treated with saline. αSMA staining was detected in cells located in the sinusoids corresponding to activated hepatic stellate cells (upper arrow) as well as in myofibroblasts around proliferating bile ducts (lower arrow).

(F). Quantification of the area with αSMA staining. Statistical analysis: *P<0.05 vs sham; #P<0.05 vs saline, MP6PHSA and oral losartan.

SMA staining on liver sections from CCl4 animals treated with losartan-ULS-M6PHSA showed less accumulation of αSMA-positive cells (H) compared to diseased animals treated with saline (G).

(I). Quantification of the area with αSMA staining in liver specimens *P<0.05 vs saline.

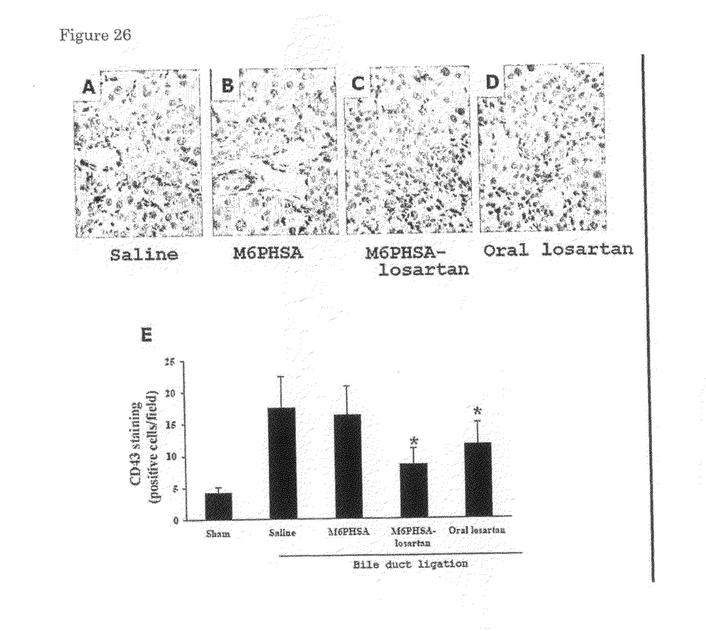

FIG. 26. Effect of different treatments on the infiltration of inflammatory cells in the hepatic parenchyma, as assessed by CD43 staining. BDL rats receiving saline (A) or M6PHSA (B) showed intense infiltration by CD43-positive cells (brown staining). Treatment with losartan-ULS-M6PHSA (C), and in a lesser extent losartan (D) reduced the number of CD43 infiltrating leukocytes (magnification 4×).

E). Quantification of the number of positive cells in 20 randomly chosen high power fields. *P<0.05 vs saline and M6PHSA.

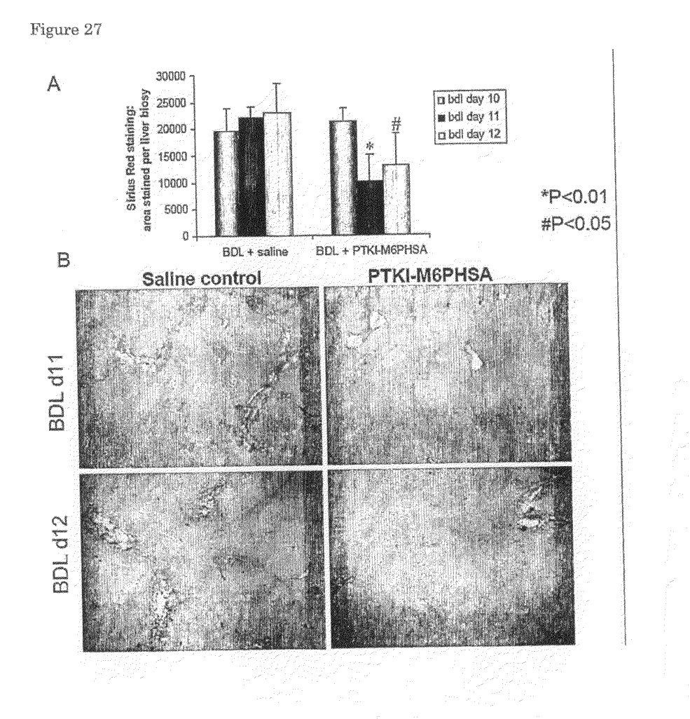

FIG. 27. Effect of PTKI-cisULS-M6PHSA on the deposition of collagen in the liver, as assessed by Sirius Red staining.

A. Quantification of the area with Sirius Red staining in rat liver specimens *P<0.05 vs BDL saline control.

B. Representative images (magnification 40×) of liver sections stained with Sirius Red. Rats receiving saline (left column) showed a marked deposition of collagen, which co-localized with areas with active fibrogenesis. Rats treated with PTKI-cisULS-M6PHSA (right column) showed attenuated liver fibrosis development at day 11 and 12 post BDL (i.e. 24 h and 48 after administration, respectively).

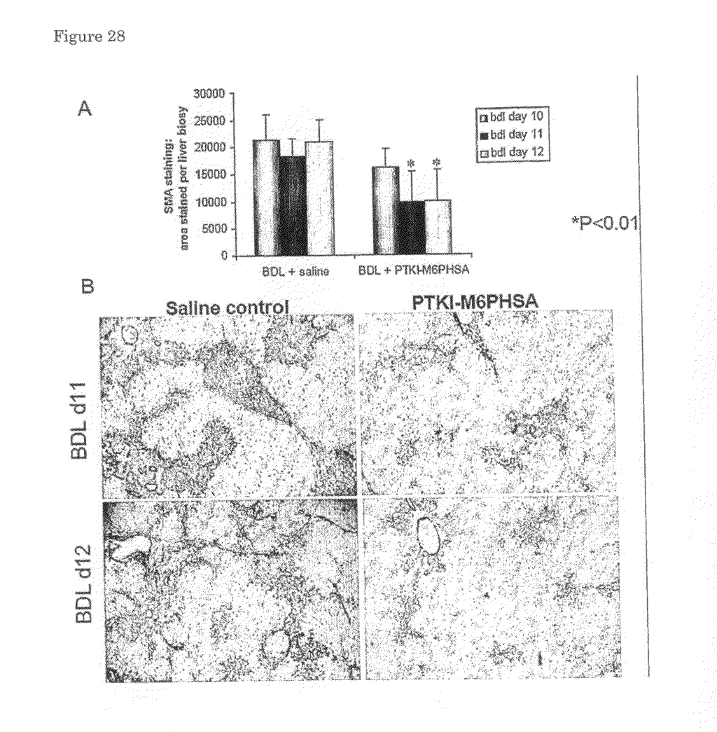

FIG. 28. Effect of PTKI-cisULS-M6PHSA on the accumulation of myofibroblasts and activated HSC, as assessed by expression of αSMA.

A. Quantification of αSMA staining. *P<0.05 vs BDL saline control.

B. Representative images (magnification 40×) of rats receiving saline (left panels) or PTKI-cisULS-M6PHSA (right panels). Treatment with a single dose of PTKI-cisULS-M6PHSA at day 10 post BDL reduced the number of myofibroblasts at day 11 and 12 post BDL (i.e. 24 h and 48 after administration, respectively).

### 3.2.2. Evaluation of Kidney-Directed Cell Targeting Complexes.

In order to detect potential platinum-related toxicity of kidney directed cell targeting complexes, we evaluated SB202190-cisULS-LZM in healthy rats.

Male Wistar rats were treated with SB202190-cisULS-LZM as described above and compared to untreated animals and to animals treated with a low dose of cisplatin (3 mg/kg, i.v.) for 24 h. The dose of cisplatin used is however 8-fold higher than the amount of platinum in the SB202190-cisULS-LZM conjugate. To examine the effect on renal function, serum and urine creatinine levels were determined and creatinine clearances were calculated. In case of SB202190-cisULS-LZM treated animals, creatinine clearance at 24, 32, 48 and 72 h was calculated from serum and urine samples collected at the latest 24 h before sacrificing the animals. To calculate proteinuria for the SB202190-cisULS-LZM group, the mean of the urinary protein levels at different days was taken. TUNEL stainings were performed on frozen kidney sections to examine the number of apoptotic cells. In addition, we determined the platinum levels in kidneys using Inductively Coupled Plasma Atomic Absorption Spectrometry (ICP-AAS) after digestion of the tissue in concentrated nitric acid for 24 h at room temperature and heating at 70° C. until the formation of clear solution. The results are summarized in Table 1.

TABLE 1
Pharmacological evaluation of SB202190-cisULS-LZM in
normal rats. Comparison of renal function parameters,
induction of apoptosis and renal platinum content.

| | Untreated | SB-ULS-LZM- | Cisplatin- |
|---|---|---|---|
| Parameters | group | treated group | treated group |
| Creatinine | 1.0 ± 0.13 | 0.8 ± 0.03 | 1.1 ± 0.13 |
| clearance | | | |
| (ml/min) | | | |
| Urinary protein | 19.16 ± 1.3 | 29.6 ± 2.0†† | 23 ± 3.6 |
| levels (mg/d) | | | |
| TUNEL positive | 46.6 ± 4.14 | 42.7 ± 18** | 187 ± 26†† |
| cells (numbers | | | |
| per field) | | | |
| Renal platinum | N.D. | 37.0 ± 1.2** | 28.4 ± 0.8 |
| concentration | | | |
| (nmol/g) | | | |
| Change in body | −1.75 ± 2.25 | +0.3 ± 1.2* | −5.0 ± 0.6†† |
| weight (%) | | | |

Data for unreacted and cisplatin groups are represented as mean ± SEM, n = 4 at 24 h.
The values for SB-ULS-LZM are shown as mean ± SEM, n = 4 at 24, 32, 48 and 72 h except for TUNEL positive cells which is n = 5 at 24 (n = 2), 32, 48 and 72 h.
The differences between cisplatin and SB-ULS-LZM groups are
*p, 0.05,
**p, 0.01.
The differences versus untreated groups are
†p, 0.05,
††p < 0.01.
N.D., not detectable.

Creatinine clearance remained normal in both groups while an increase in urinary protein levels was found with SB202190-ULS-LZM in comparison to untreated rats. However, the observed value of the conjugate treated group was well within normal limits. Second, we investigated tubular cell apoptosis by TUNEL staining of kidney sections, which clearly demonstrated cisplatin-induced tubular toxicity as compared to untreated rats. In contrast, SB-ULS-LZM treated rats showed no increase in the number of apoptotic cells, despite the comparable levels of platinum in the kidneys of both treated groups.

The potential antifibrotic and anti-inflammatory activity of kidney directed cell targeting complexes was evaluated in two different models of renal disease, the unilateral ischemia/reperfusion injury (I/R) model and the unilateral ureteral obstruction (UUO) model.

### Evaluation of Cell Targeting Complexes in the I/R Model

We hypothesized that treatment with kidney directed cell targeting complexes would afford sufficient amounts of active drug in the kidney during a prolonged period, due to their controlled drug release profiles as discussed above. Conjugate were therefore administered 2 h before ischemia so that they could accumulate completely within the kidneys. After the administration of compounds, animals were placed back into the cages until the induction of renal ischemia. Rats were operated and the renal artery and vein were clamped under microscope to stop renal blood flow. After 45 min, clamps were removed and reperfusion of the kidney was observed before closing of the wound. Sham-operated animals underwent a similar surgical procedure except for the clamping of blood vessels. After 4 days, animals were sacrificed and blood samples were collected from the abdominal aorta. Kidneys were isolated after gently flushing the organs with saline and preserved in 4% formalin for preparation of paraffin embedded sections, or frozen in ice-cold isopentane for preparation of cryosections. Immunostainings were performed according to generally established methods and quantified by morphometric analysis or by semi-quantitative scoring a blind manner by two independent observers.

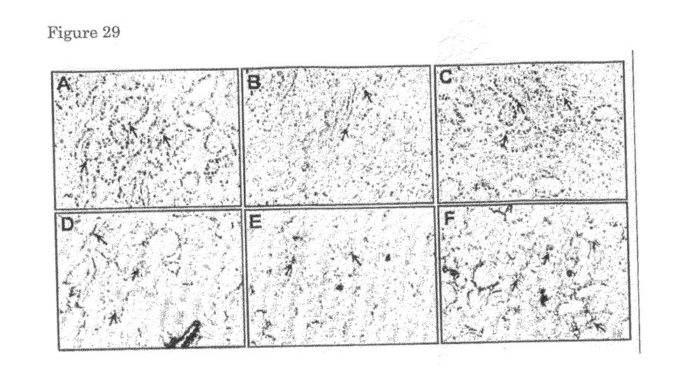

Results obtained with SB202190-cisULS-LZM are shown in FIG. 29. Animals were treated with SB202190-cisULS-LZM (32 mg/kg of conjugate, equivalent to 752 μg/kg SB202190; n=6), vehicle (5% glucose; n=6), or free SB202190 (800 μg/kg; n=3). SB202190-cisULS-LZM was dissolved in 5% glucose whereas SB202190 was dissolved in 20% hydroxypropyl-β-cyclodextrin solution with 5% DMSO. Anti-p-p38 immunohistochemical stainings (antibody obtained from cell signaling) were performed on paraffin embedded sections. αSMA stainings were performed on cryostat sections. Vehicle treated I/R rats had dilated and damaged tubules that strongly showed p-p38 positive cells (panel A, grade +++) in both renal cortex and medulla. Treatment with SB202190-ULS-LZM reduced the number of p-p38 positive cells in the medulla (panel B, grade +) but no reduction was found in SB202190 treated animals (C, grade +++). Moreover, we found that after 4 days of I/R injury, SMA expression was highly increased in the tubulointerstitial space of the renal cortex (panel D, grade +++). A single dose of SB202190-ULS-LZM conjugate reduced SMA expression (panel E, grade ++) whereas non-targeted SB202190 did not affect the expression of SMA (panel F, grade +++).

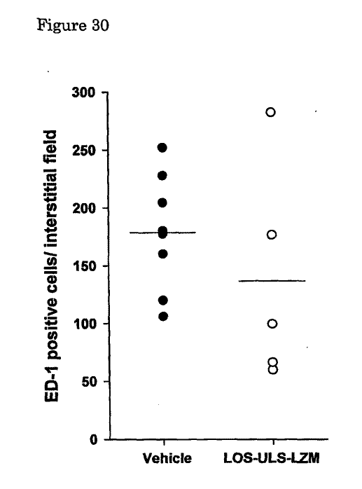

FIG. 30 shows the results obtained after treatment of I/R rats with losartan-cisULS-LZM. Animals that were subjected to I/R procedure as described above were treated with four doses of losartan-cisULS-LZM (20 mg/kg/day, equivalent to 520 μg losartan/kg/day). The first dose was administered 2 h before infliction of I/R damage and animals were sacrificed 24 h after the last dose. To detect the number of infiltrated macrophages, paraffin-embedded kidney sections were incubated with an antibody against the rat macrophage marker ED-1. The extent of interstitial macrophage influx was determined by computerized morphometry. The amount of brown precipitate was measured and represented as either number of macrophages in the selected area. All morphometric measurements were performed by a blinded observer. As can be observed, treatment with losartan-cisULS-LZM reduced the number of infiltrated macrophages.

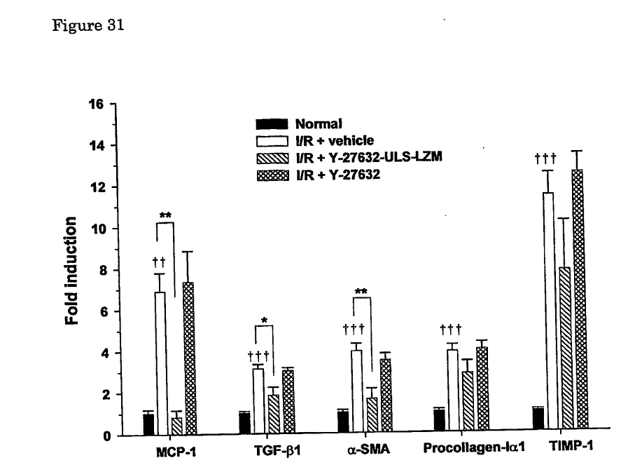

A third cell targeting complex evaluated in the unilateral I/R model is the Y27632-cisULS-LZM conjugate. Similar to losartan-cisULS-LZM, I/R animals were treated with four intravenous doses of the product (20 mg/kg of Y27632-cisULS-LZM equivalent to 555 μg/kg of Y27632, dissolved in 5% glucose). A control group of animals was treated with free Y27632 (555 μg/kg). The first dose was administered 2 h before infliction of I/R damage and animals were sacrificed 24 h after the last dose, i.e. at day 4 after I/R. Kidney pieces were snap-frozen in liquid nitrogen for mRNA isolation and quantitative gene-expression analysis. Total RNA was isolated from renal cortex using Bio-Rad's Aurum Total RNA Mini kit (Bio-Rad, Hercules, Calif.). RNA content was measured by a nanodrop UV-detector (NanoDrop Technologies, Wilmington, Del.). cDNA was synthesized from similar amounts of RNA using the Superscript III first strand synthesis kit (Invitrogen, Carlsbad, Calif.). Gene expression levels for the following genes were measured by quantitative real-time RT-PCR (Applied Biosystems, Foster City, Calif.). The primers for rat species were obtained from Sigma-Genosys (Haverhill, UK) as follows: monocyte chemoattractant protein-1 (MCP-1; 5′-TCC TCC ACC ACT ATG CAG GT-3′ and 5′-TTC CTT ATT GGG GTC AGC AC-3′, 255 bp), tissue inhibitor of metalloproteinase-1 (TIMP-1; 5′-GAG AGC CTC TGT GGA TAT GT-3′ and 5′-CAG CCA GCA CTA TAG GTC TT-3′, 334 bp), procollagen-Iα1 (5′-AGC CTG AGC CAG CAG ATT GA-3′ and 5′-CCA GGT TGC AGC CTT GGT TA-3′, 145 bp), alpha smooth muscle actin (α-SMA; 5′-GAC ACC AGG GAG TGA TGG TT-3′ and 5′-GTT AGC AAG GTC GGA TGC TC-3′, 202 bp), TGF-β1 (5′-ATA CGC CTG AGT GGC TGT CT and 5′-TGG GAC TGA TCC CAT TGA TT-3′, 153 bp) and Glyceraldehyde-3-phosphate dehydrogenase (GAPDH; 5′-CGC TGG TGC TGA GTA TGT CG-3′ and 5′-CTG TGG TCA TGA GCC CTT CC-3′, 179 bp). SYBR® Green PCR Master Mix (Applied Biosystems, Warrington, UK) was used as a fluorescent probe for real-time RT-PCR. For each sample, 1 μl of cDNA was mixed with 0.4 μl of each gene-specific primer (50 μM), 0.8 μl DMSO, 8.4 μl water and 10 μl SYBR Green PCR Master Mix. The cDNA amplification was performed until 40 cycles followed by dissociation cycle. The final product was examined to provide a single peak in the dissociation curve. Finally, the threshold cycle number (Ct) was calculated for each gene and relative gene expressions were calculated after normalizing for the expression of the control gene GAPDH. FIG. 31 presents the gene expression levels in the kidney. In comparison to normal rats, vehicle-treated I/R animals had a significant increase in the gene expression of the inflammation marker MCP-1 and of fibrosis markers α-SMA, TGF-β1, procollagen-Iα1 and TIMP-1. Data represents mean±SEM. ††p<0.01 and †††p<0.001 represent the difference versus normal rats. Other differences are indicated as *p<0.05 and ** p<0.01.

### Evaluation of a Cell Targeting Complex in the UUO Model

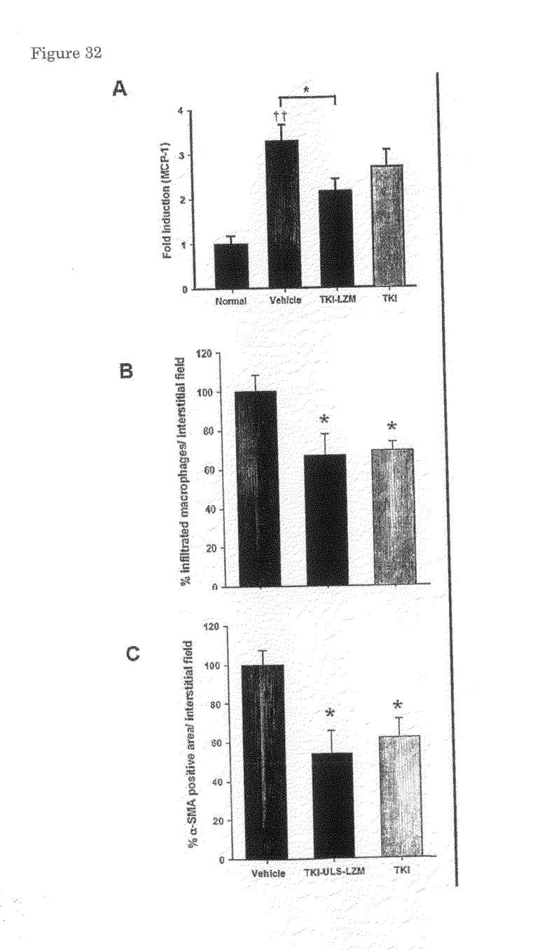

The efficacy of TKI-cisULS-LZM was studied after a single dose of the compound in the UUO model of renal fibrosis. Animals were divided into 4 groups: normal rats, fibrotic control animals (vehicle, 5% glucose), TKI-LZM (25 mg/kg equiv. to 630 μg/kg TKI) and free, unconjugated TKI (630 μg/kg). TKI was dissolved in 20% hydroxylpropyl-β-cyclodextrin in water with 5% DMSO, whereas TKI-LZM was dissolved in 5% glucose. To allow unhindered uptake of the products in the kidneys, rats were injected intravenously with either of these compounds 2 h before the uretheral obstruction. Left kidneys and ureter were exposed via a flank-incision under isoflurane anesthesia after which the ureter was ligated at three sites with 4-0 silk near the hilum. After 3 days, animals were sacrificed under anesthesia and kidneys were gently flushed and harvested. Kidney cortex pieces were snap-frozen to isolate RNA as described above. Kidney pieces were fixed in 4% formalin solution in PBS to make paraffin-embedded sections for anti-α-SMA and ED-1 immunohistochemical staining and morphometric analysis. Results of the study are shown in FIG. 32. Treatment with TKI-cisULS-LZM substantially reduced the MCP-1 gene expression (panel A). In contrast, a single dose of free TKI did not lower the expressions of MCP-1. Immunohistochemical analyses confirmed the gene-expression data. In line with the lowered production of MCP-1, which is a chemoattractant for macrophages, we also detected significant reduced levels of infiltrated macrophages upon TKI-LZM treatment (ED-1 immunostaining, panel B). However, in contrast to the gene expression study, we also found a reduction of ED-1 immunostaining with free TKI. Moreover, the expression of the fibrosis marker α-SMA was also significantly decreased by TKI-LZM or TKI treatments, which indicates potential antifibrotic activity of TKI and TKI-LZM (panel C). These data demonstrate the potential antifibrotic activity of TKI-cisULS-LZM in renal fibrosis.

### REFERENCES

[1] L. Beljaars, G. Molema, B. Weert, H. Bonnema, P. Olinga, G. M. M. Groothuis, D. K. F. Meijer, and K. Poelstra, Albumin modified with mannose 6-phosphate: A potential carrier for selective delivery of antifibrotic drugs to rat and human hepatic stellate cells, Hepatology 295 (1999) 1486-1493.
[2] L. Beljaars, P. Olinga, G. Molema, P. De Bleser, A. Geerts, G. M. M.
Groothuis, D. K. F. Meijer, and K. Poelstra, Characteristics of the hepatic stellate cell-selective carrier mannose 6-phosphate modified albumin (M6P28-HSA), Liver 215 (2001) 320-328.
[3] R. J. Kok, A. J. Schraa, E. J. Bos, H. E. Moorlag, S. A. Asgeirsdottir, M. Everts, D. K. F. Meijer, and G. Molema, Preparation and functional evaluation of RGD-modified proteins as alpha(v) beta(3) integrin directed therapeutics, Bioconjugate Chemistry 13 (2002) 128-135.
[4] J. Prakash, A. van Loenen-Weemaes, M. Haas, J. H. Proost, D. K. F. Meijer, F. Moolenaar, K. Poelstra, and R. J. Kok, Renal-selective delivery and ACE inhibition by subcutaneously administered captopril-lysozyme. Drug Metabolism and Disposition 33 (2005) 683-688.
[5] A. Geerts, T. Niki, K. Hellemans, D. De Craemer, K. van den Berg, J.-M. Lazou, G. Stange, M. van de Winkel, and P. J. De Bleser, Purification of rat hepatic stellate cells by side scatter-activated cell sorting, Hepatology 27 (1998) 590-598.
[6] Claria J & Jimenez, W. Renal dysfunction and ascites in carbon tetrachloride-induced liver cirrhosis in rats. Pathogenesis, diagnosis and treatment. (eds. Arroyo, V., Gines, P., Rodes, J. & Schrier, R.) 379-396 (Blackwell Science Inc. Malden. M; USA, USA, 1999).
[7] Prakash J, Saluja V, Visser J, Moolenaar F, Meijer D K F, Poelstra K and Kok R J (2005b) Bioanalysis and pharmacokinetics of the p38 MAPkinase inhibitor SB202190 in rats. Journal of Chromatography B 826:220-225.
[8] Chen X, Park R, Shahinian A H, Tohme M, Khankaldyyan V, Bozorgzadeh M H, Bading J R, Moats R, Laug W E and Conti P S (2004) 18F-labeled RGD peptide: initial evaluation for imaging brain tumor angiogenesis. Nucl Med Biol. 31(2):179-89.

1. A cell-targeting complex comprising a targeting moiety and a deliverable compound, wherein said targeting moiety and said deliverable compound are joined by means of a (transition) metal ion complex having at least a first reactive moiety for forming a coordination bond with a reactive site of said targeting moiety and having at least a second reactive moiety for forming a coordination bond with a reactive site of said deliverable compound, and wherein said deliverable compound is a therapeutic or diagnostic compound.

2. Cell-targeting complex according to claim 1, wherein said metal ion complex is a platinum complex.

3. Cell-targeting complex according to claim 2, wherein said platinum complex is a cis-platinum complex; preferably a cis-platinum complex comprising an inert bidentate moiety as a stabilising bridge.

4. Cell-targeting complex according to any one of the preceding claims, wherein said targeting moiety comprises one or more members of a binding pair, preferably (a part of an antibody or a derivative thereof, a hapten or a receptor ligand.

5. Cell-targeting complex according to any one of the preceding claims, wherein said targeting moiety comprises a macromolecular carrier loaded with at least one member of a binding pair, said macromolecular carrier preferably being avidin or HSA.

6. Cell-targeting complex according to claim 5, wherein said at least one member of a binding pair is a receptor ligand, preferably a cyclic RGD peptide or Mannose-6-phosphate.

7. Cell-targeting complex according to claim 5 or 6, wherein said macromolecular carrier comprising at least one polyethyleneglycol (PEG) moiety, either as linking moiety between the ligand and the carrier or as tethered moiety providing shielding functionality.

8. Cell-targeting complex according to any one of the preceding claims, wherein said targeting moiety comprises a low molecular weight protein that accumulates in the kidney, the liver or a tumor of a mammal, including a human, said low molecular weight protein preferably being selected from lysozyme, alkaline phosphatase, superoxide dismutase, immunoglobulins or parts thereof (e.g. single chain, Fab-fragments or whole IgG).

9. Cell-targeting complex according to any one of the preceding claims, wherein said deliverable compound inhibits a signal transduction cascade in a cellular system, or has anti-inflammatory activity, anti-hypertensive activity, antifibrotic activity, anti-angiogenic activity, antitumor activity or apoptosis-inducing activity.

10. Cell-targeting complex according to any one of the preceding claims, wherein said deliverable compound is an anti-inflammatory compound, preferably pentoxifylline or pyrrolidine dithiocarbamate (PDTC).

11. Cell-targeting complex according to any one of claims 1-9, wherein said deliverable compound is an anti-hypertensive agent, preferably Losartan.

12. Cell-targeting complex according to any one of claims 1-9, wherein said deliverable compound is a kinase inhibitor, preferably PTKI, SB202190, PTK787, TKI, Y27632 or AG1295.

13. Cell-targeting complex according to any one of claims 1-9, wherein said deliverable compound is a protein.

14. Cell-targeting complex according to claim 13, wherein said protein comprises at least one residue selected from histidine, cysteine and methionine, preferably selected from histidine and methionine.

15. Cell-targeting complex according to claim 13 or 14, wherein said protein is selected from one of the groups consisting of:
a) cytokines and growth factors;
b) therapeutic proteins such as TNF-related apoptosis-inducing ligand (TRAIL), or
c) IL-10, alkaline phosphatase, superoxide dismutase, immunoglobulins or parts thereof (e.g. single chain, Fab-fragments or whole IgG), lactoferrin, xanthine oxidase, or TNFα.

16. A cell-targeting complex according to any one of claims 1-15, wherein said deliverable compound is a therapeutic compound, for use as a medicament.

17. A pharmaceutical composition comprising a cell-targeting complex as defined in any one of claims 1-15, and a pharmaceutically acceptable carrier.

18. Use of a (transition) metal ion complex capable of forming coordination bonds for linking a targeting moiety to a deliverable compound.

19. Use according to claim 18, wherein said metal ion complex is a platinum complex.

20. Use according to claim 19, wherein said platinum complex is a cis-platinum complex, preferably comprising an inert bidentate moiety as a stabilising bridge.

21. Use according to any one of claims 18-20, wherein said deliverable compound is a therapeutic compound.

22. Use according to claim 21, wherein said therapeutic compound inhibits a signal transduction cascade in a cellular system, or has anti-inflammatory, anti-hypertensive, antifibrotic, anti-angiogenic, antitumor or apoptosis-inducing activity.

23. Use according to claim 21 or 22, wherein said therapeutic compound is pentoxifylline, PDTC, Losartan, PTKI, SB202190, PTK787, TKI, Y27632, AG1295 or TRAIL.

24. Method of coupling a targeting moiety to a deliverable compound comprising providing a (transition) metal ion complex having at least a first reactive moiety for forming a coordination bond with a reactive site of said targeting moiety and having at least a second reactive moiety for forming a coordination bond with a reactive site of said deliverable compound and allowing said (transition) metal ion complex to form coordination bonds with said targeting moiety and said deliverable compound.

25. Method according to claim 24, wherein said metal ion complex is a platinum complex.

26. Method according to claim 25, wherein said platinum complex is a cis-platinum complex, preferably comprising an inert bidentate moiety as a stabilising bridge.

27. Method according to any one of claims 24-26, wherein said targeting moiety is a member of a binding pair, preferably an antibody, a hapten or a receptor ligand.

28. Method according to any one of claims 24-27, wherein said deliverable compound is a therapeutic compound.

29. Method according to claim 28, wherein said therapeutic compound inhibits a signal transduction cascade in a cellular system, or has anti-inflammatory, anti-hypertensive, antifibrotic, anti-angiogenic, antitumor or apoptosis-inducing activity.

30. Method according to claim 28 or 29, wherein said therapeutic compound is pentoxifylline, PDTC, Losartan, PTKI, SB202190, PTK787, TKI, Y27632, AG1295 or TRAIL.

31. A method of targeting therapeutic compounds to selected cell populations comprising administering to a subject in need thereof a therapeutically effective amount of a pharmaceutical composition as defined in claim 17.

32. Method according to claim 31, wherein said cell populations are cells from the kidney, the liver or a tumor in a subject.

33. Method according to claim 31 or 32, wherein said therapeutic compound is selected from
a) anti-inflammatory compounds, preferably pentoxifylline and PDTC;
b) anti-hypertensive agents, preferably Losartan;
c) kinase inhibitors, preferably PTKI, SB202190, PTK787, TKI, Y27632 and AG1295.
